SEQUENCE LISTING (235 sequences)

SEQ ID NO: 1 - protein, 5 aa, Artificial Sequence
  source: /note=“Description of Artificial Sequence: Synthetic peptide”
TYWMH

SEQ ID NO: 2 - protein, 17 aa, Artificial Sequence
  source: /note=“Description of Artificial Sequence: Synthetic peptide”
NIYPGTGGSNFDEKFKN

SEQ ID NO: 3 - protein, 8 aa, Artificial Sequence
  source: /note=“Description of Artificial Sequence: Synthetic peptide”
WTTGTGAY

SEQ ID NO: 4 - protein, 7 aa, Artificial Sequence
  source: /note=“Description of Artificial Sequence: Synthetic peptide”
GYTFTTY

SEQ ID NO: 5 - protein, 6 aa, Artificial Sequence
  source: /note=“Description of Artificial Sequence: Synthetic peptide”
YPGTGG

SEQ ID NO: 6 - protein, 117 aa, Artificial Sequence
  source: /note=“Description of Artificial Sequence: Synthetic polypeptide”
QVQLQQPGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTLVTVSA

SEQ ID NO: 7 - DNA, 351 bp, Artificial Sequence
  source: /note=“Description of Artificial Sequence: Synthetic polynucleotide”
caggtccagctgcagcaacctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccaagggactctggtcactgtctctgca

SEQ ID NO: 8 - protein, 117 aa, Artificial Sequence
  source: /note=“Description of Artificial Sequence: Synthetic polypeptide”
QVQLQQSGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTLVTVSA

SEQ ID NO: 9 - DNA, 351 bp, Artificial Sequence
  source: /note=“Description of Artificial Sequence: Synthetic polynucleotide”
caggtccagctgcagcagtctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccaagggactctggtcactgtctctgca

SEQ ID NO: 10 - protein, 17 aa, Artificial Sequence
  source: /note=“Description of Artificial Sequence: Synthetic peptide”
KSSQSLLDSGNQKNFLT

SEQ ID NO: 11 - protein, 7 aa, Artificial Sequence
  source: /note=“Description of Artificial Sequence: Synthetic peptide”
WASTRES

SEQ ID NO: 12 - protein, 9 aa, Artificial Sequence
  source: /note=“Description of Artificial Sequence: Synthetic peptide”
QNDYSYPCT

SEQ ID NO: 13 - protein, 13 aa, Artificial Sequence
  source: /note=“Description of Artificial Sequence: Synthetic peptide”
SQSLLDSGNQKNF

SEQ ID NO: 14 - protein, 3 aa, Artificial Sequence
  source: /note=“Description of Artificial Sequence: Synthetic peptide”
WAS

SEQ ID NO: 15 - protein, 6 aa, Artificial Sequence
  source: /note=“Description of Artificial Sequence: Synthetic peptide”
DYSYPC

SEQ ID NO: 16 - protein, 113 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPCTFGGGTKLEIK

SEQ ID NO: 17 - DNA, 339 bp, Artificial Sequence
  source: /note=“Description of Artificial Sequence: Synthetic polynucleotide”
gacattgtgatgacccagtctccatcctccctgactgtgacagcaggagagaaggtcactatgagctgcaagtccagtcagagtctgttagacagtggaaatcaaaagaacttcttgacctggtaccagcagaaaccagggcagcctcctaaactgttgatcttctgggcatccactagggaatctggggtccctgatcgcttcacaggcagtggatctgtaacagatttcactctcaccatcagcagtgtgcaggctgaagacctggcagtttattactgtcagaatgattatagttatccgtgcacgttcggaggggggaccaagctggaaataaaa

SEQ ID NO: 18 - protein, 117 aa, Artificial Sequence
  source: /note=“Description of Artificial Sequence: Synthetic polypeptide”
QVQLQQPGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTTVTVSS

SEQ ID NO: 19 - DNA, 351 bp, Artificial Sequence
  source: /note=“Description of Artificial Sequence: Synthetic polynucleotide”
caggtccagctgcagcagcctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccagggcaccaccgtgaccgtgtcctcc

SEQ ID NO: 20 - protein, 444 aa, Artificial Sequence
  source: /note=“Description of Artificial Sequence: Synthetic polypeptide”
QVQLQQPGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - DNA, 1332 bp, Artificial Sequence
  source: /note=“Description of Artificial Sequence: Synthetic polynucleotide”
caggtccagctgcagcagcctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 22 - protein, 117 aa, Artificial Sequence
  source: /note=“Description of Artificial Sequence: Synthetic polypeptide”
QVQLQQSGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTTVTVSS

SEQ ID NO: 23 - DNA, 351 bp, Artificial Sequence
  source: /note=“Description of Artificial Sequence: Synthetic polynucleotide”
caggtccagctgcagcagtctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccagggcaccaccgtgaccgtgtcctcc

SEQ ID NO: 24 - protein, 113 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPCTFGQGTKVEIK

SEQ ID NO: 25 - DNA, 339 bp, Artificial Sequence
  source: /note=“Description of Artificial Sequence: Synthetic polynucleotide”
gacattgtgatgacccagtctccatcctccctgactgtgacagcaggagagaaggtcactatgagctgcaagtccagtcagagtctgttagacagtggaaatcaaaagaacttcttgacctggtaccagcagaaaccagggcagcctcctaaactgttgatcttctgggcatccactagggaatctggggtccctgatcgcttcacaggcagtggatctgtaacagatttcactctcaccatcagcagtgtgcaggctgaagacctggcagtttattactgtcagaatgattatagttatccgtgcacgttcggccaagggaccaaggtggaaatcaaa

SEQ ID NO: 26 - protein, 220 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPCTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 660 bp, Artificial Sequence
  source: /note=“Description of Artificial Sequence: Synthetic polynucleotide”
gacattgtgatgacccagtctccatcctccctgactgtgacagcaggagagaaggtcactatgagctgcaagtccagtcagagtctgttagacagtggaaatcaaaagaacttcttgacctggtaccagcagaaaccagggcagcctcctaaactgttgatcttctgggcatccactagggaatctggggtccctgatcgcttcacaggcagtggatctgtaacagatttcactctcaccatcagcagtgtgcaggctgaagacctggcagtttattactgtcagaatgattatagttatccgtgcacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 28-29 - skipped (2 entries)

SEQ ID NO: 30 - protein, 444 aa, Artificial Sequence
  source: /note=“Description of Artificial Sequence: Synthetic polypeptide”
QVQLQQSGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - DNA, 1332 bp, Artificial Sequence
  source: /note=“Description of Artificial Sequence: Synthetic polynucleotide”
caggtccagctgcagcagtctgggtctgagctggtgaggcctggagcttcagtgaagctgtcctgcaaggcgtctggctacacattcaccacttactggatgcactgggtgaggcagaggcctggacaaggccttgagtggattggaaatatttatcctggtactggtggttctaacttcgatgagaagttcaaaaacaggacctcactgactgtagacacatcctccaccacagcctacatgcacctcgccagcctgacatctgaggactctgcggtctattactgtacaagatggactactgggacgggagcttattggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 32 - protein, 9 aa, Artificial Sequence
  source: /note=“Description of Artificial Sequence: Synthetic peptide”
QNDYSYPYT

SEQ ID NO: 33 - protein, 6 aa, Artificial Sequence
  source: /note=“Description of Artificial Sequence: Synthetic peptide”
DYSYPY

SEQ ID NO: 34 - protein, 113 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPYTFGQGTKVEIK

SEQ ID NO: 35 - DNA, 339 bp, Artificial Sequence
  source: /note=“Description of Artificial Sequence: Synthetic polynucleotide”
gacattgtgatgacccagtctccatcctccctgactgtgacagcaggagagaaggtcactatgagctgcaagtccagtcagagtctgttagacagtggaaatcaaaagaacttcttgacctggtaccagcagaaaccagggcagcctcctaaactgttgatcttctgggcatccactagggaatctggggtccctgatcgcttcacaggcagtggatctgtaacagatttcactctcaccatcagcagtgtgcaggctgaagacctggcagtttattactgtcagaatgattatagttatccgtacacgttcggccaagggaccaaggtggaaatcaaa

SEQ ID NO: 36 - protein, 220 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 660 bp, Artificial Sequence
  source: /note=“Description of Artificial Sequence: Synthetic polynucleotide”
gacattgtgatgacccagtctccatcctccctgactgtgacagcaggagagaaggtcactatgagctgcaagtccagtcagagtctgttagacagtggaaatcaaaagaacttcttgacctggtaccagcagaaaccagggcagcctcctaaactgttgatcttctgggcatccactagggaatctggggtccctgatcgcttcacaggcagtggatctgtaacagatttcactctcaccatcagcagtgtgcaggctgaagacctggcagtttattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 38 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 39 - DNA, 351 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactgggtgcgacaggccactggacaagggcttgagtggatgggtaatatttatcctggtactggtggttctaacttcgatgagaagttcaagaacagagtcacgattaccgcggacaaatccacgagcacagcctacatggagctgagcagcctgagatctgaggacacggccgtgtattactgtacaagatggactactgggacgggagcttattggggccagggcaccaccgtgaccgtgtcctcc

SEQ ID NO: 40 - protein, 444 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - DNA, 1332 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactgggtgcgacaggccactggacaagggcttgagtggatgggtaatatttatcctggtactggtggttctaacttcgatgagaagttcaagaacagagtcacgattaccgcggacaaatccacgagcacagcctacatggagctgagcagcctgagatctgaggacacggccgtgtattactgtacaagatggactactgggacgggagcttattggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 42 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTEFTLTISSLQPDDFATYYCQNDYSYPYTFGQGTKVEIK

SEQ ID NO: 43 - DNA, 339 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtaccagcagaaacctggccaggctcccaggctcctcatctattgggcatccactagggaatctggggtcccatcaaggttcagcggcagtggatctgggacagaattcactctcaccatcagcagcctgcagcctgatgattttgcaacttattactgtcagaatgattatagttatccgtacacgttcggccaagggaccaaggtggaaatcaaa

SEQ ID NO: 44 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTEFTLTISSLQPDDFATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 660 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtaccagcagaaacctggccaggctcccaggctcctcatctattgggcatccactagggaatctggggtcccatcaaggttcagcggcagtggatctgggacagaattcactctcaccatcagcagcctgcagcctgatgattttgcaacttattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 46 - protein, 113 aa, Artificial Sequence
  source: /note=“Description of Artificial Sequence: Synthetic polypeptide”
DIQMTQSPSSLSASVGDRVTITCKSSQSLLDSGNQKNFLTWYQQKPGQAPRLLIYWASTRESGIPPRFSGSGYGTDFTLTINNIESEDAAYYFCQNDYSYPYTFGQGTKVEIK

SEQ ID NO: 47 - DNA, 339 bp, Artificial Sequence
  source: /note=“Description of Artificial Sequence: Synthetic polynucleotide”
gacatccagatgacccagtctccatcctccctgtctgcatctgtaggagacagagtcaccatcacttgcaagtccagtcagagtctgttagacagtggaaatcaaaagaacttcttgacctggtaccagcagaaacctggccaggctcccaggctcctcatctattgggcatccactagggaatctgggatcccacctcgattcagtggcagcgggtatggaacagattttaccctcacaattaataacatagaatctgaggatgctgcatattacttctgtcagaatgattatagttatccgtacacgttcggccaagggaccaaggtggaaatcaaa

SEQ ID NO: 48 - protein, 220 aa, Artificial Sequence
  source: /note=“Description of Artificial Sequence: Synthetic polypeptide”
DIQMTQSPSSLSASVGDRVTITCKSSQSLLDSGNQKNFLTWYQQKPGQAPRLLIYWASTRESGIPPRFSGSGYGTDFTLTINNIESEDAAYYFCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - DNA, 660 bp, Artificial Sequence
  source: /note=“Description of Artificial Sequence: Synthetic polynucleotide”
gacatccagatgacccagtctccatcctccctgtctgcatctgtaggagacagagtcaccatcacttgcaagtccagtcagagtctgttagacagtggaaatcaaaagaacttcttgacctggtaccagcagaaacctggccaggctcccaggctcctcatctattgggcatccactagggaatctgggatcccacctcgattcagtggcagcgggtatggaacagattttaccctcacaattaataacatagaatctgaggatgctgcatattacttctgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 50 - protein, 117 aa, Artificial Sequence
  source: /note=“Description of Artificial Sequence: Synthetic polypeptide”
EVQLVQSGAEVKKPGESLRISCKGSGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSS

SEQ ID NO: 51 - DNA, 351 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactggatcaggcagtccccatcgagaggccttgagtggctgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttattggggccagggcaccaccgtgaccgtgtcctcc

SEQ ID NO: 52 - protein, 444 aa, Artificial Sequence
  source: /note=“Description of Artificial Sequence: Synthetic polypeptide”
EVQLVQSGAEVKKPGESLRISCKGSGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - DNA, 1332 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactggatcaggcagtccccatcgagaggccttgagtggctgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttattggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 54 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 55 - DNA, 339 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtatcagcagaaaccagggaaagctcctaagctcctgatctattgggcatccactagggaatctggggtcccatcaaggttcagtggaagtggatctgggacagattttactttcaccatcagcagcctgcagcctgaagatattgcaacatattactgtcagaatgattatagttatccgtacacgttcggccaagggaccaaggtggaaatcaaa

SEQ ID NO: 56 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - DNA, 660 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtatcagcagaaaccagggaaagctcctaagctcctgatctattgggcatccactagggaatctggggtcccatcaaggttcagtggaagtggatctgggacagattttactttcaccatcagcagcctgcagcctgaagatattgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 58 - protein, 113 aa, Artificial Sequence
  source: /note=“Description of Artificial Sequence: Synthetic polypeptide”
DIVMTQTPLSLPVTPGEPASISCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 59 - DNA, 339 bp, Artificial Sequence
  source: /note=“Description of Artificial Sequence: Synthetic polynucleotide”
gatattgtgatgacccagactccactctccctgcccgtcacccctggagagccggcctccatctcctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 60 - protein, 220 aa, Artificial Sequence
  source: /note=“Description of Artificial Sequence: Synthetic polypeptide”
DIVMTQTPLSLPVTPGEPASISCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 660 bp, Artificial Sequence
  source: /note=“Description of Artificial Sequence: Synthetic polynucleotide”
gatattgtgatgacccagactccactctccctgcccgtcacccctggagagccggcctccatctcctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 62 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLEAEDAATYYCQNDYSYPYTFGQGTKVEIK

SEQ ID NO: 63 - DNA, 339 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtatcagcagaaaccagggaaagctcctaagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 64 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - DNA, 660 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtatcagcagaaaccagggaaagctcctaagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 66 - protein, 113 aa, Artificial Sequence
  source: /note=“Description of Artificial Sequence: Synthetic polypeptide”
EIVLTQSPDFQSVTPKEKVTITCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 67 - DNA, 339 bp, Artificial Sequence
  source: /note=“Description of Artificial Sequence: Synthetic polynucleotide”
gaaattgtgctgactcagtctccagactttcagtctgtgactccaaaggagaaagtcaccatcacctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 68 - protein, 220 aa, Artificial Sequence
  source: /note=“Description of Artificial Sequence: Synthetic polypeptide”
EIVLTQSPDFQSVTPKEKVTITCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - DNA, 660 bp, Artificial Sequence
  source: /note=“Description of Artificial Sequence: Synthetic polynucleotide”
gaaattgtgctgactcagtctccagactttcagtctgtgactccaaaggagaaagtcaccatcacctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 70 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 71 - DNA, 339 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 72 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 660 bp, Artificial Sequence
  source: /note=“Description of Artificial Sequence: Synthetic polynucleotide”
gaaattgtgttgacacagtctccagccaccctgtctttgtctccaggggaaagagccaccctctcctgcaagtccagtcagagtctgttagacagtggaaatcaaaagaacttcttgacctggtaccagcagaaacctggccaggctcccaggctcctcatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 74 - protein, 113 aa, Artificial Sequence
  source: /note=“Description of Artificial Sequence: Synthetic polypeptide”
DIQMTQSPSSLSASVGDRVTITCKSSQSLLDSGNQKNFLTWYLQKPGQSPQLLIYWASTRESGVPSRFSGSGSGTDFTFTISSLEAEDAATYYCQNDYSYPYTFGQGTKVEIK

SEQ ID NO: 75 - DNA, 339 bp, Artificial Sequence
  source: /note=“Description of Artificial Sequence: Synthetic polynucleotide”
gacatccagatgacccagtctccatcctccctgtctgcatctgtaggagacagagtcaccatcacttgcaagtccagtcagagtctgttagacagtggaaatcaaaagaacttcttgacctggtacctgcagaagccagggcagtctccacagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 76 - protein, 220 aa, Artificial Sequence
  source: /note=“Description of Artificial Sequence: Synthetic polypeptide”
DIQMTQSPSSLSASVGDRVTITCKSSQSLLDSGNQKNFLTWYLQKPGQSPQLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - DNA, 660 bp, Artificial Sequence
  source: /note=“Description of Artificial Sequence: Synthetic polynucleotide”
gacatccagatgacccagtctccatcctccctgtctgcatctgtaggagacagagtcaccatcacttgcaagtccagtcagagtctgttagacagtggaaatcaaaagaacttcttgacctggtacctgcagaagccagggcagtctccacagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 78 - protein, 113 aa, Artificial Sequence
  source: /note=“Description of Artificial Sequence: Synthetic polypeptide”
DVVMTQSPLSLPVTLGQPASISCKSSQSLLDSGNQKNFLTWYQQKPGKAPKLLIYWASTRESGVPSRFSGSGSGTDFTFTISSLEAEDAATYYCQNDYSYPYTFGQGTKVEIK

SEQ ID NO: 79 - DNA, 339 bp, Artificial Sequence
  source: /note=“Description of Artificial Sequence: Synthetic polynucleotide”
gatgttgtgatgactcagtctccactctccctgcccgtcacccttggacagccggcctccatctcctgcaagtccagtcagagtctgttagacagtggaaatcaaaagaacttcttaacctggtatcagcagaaaccagggaaagctcctaagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaa

SEQ ID NO: 80 - protein, 220 aa, Artificial Sequence
  source: /note=“Description of Artificial Sequence: Synthetic polypeptide”
DVVMTQSPLSLPVTLGQPASISCKSSQSLLDSGNQKNFLTWYQQKPGKAPKLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 660 bp, Artificial Sequence
  source: /note=“Description of Artificial Sequence: Synthetic polynucleotide”
gatgttgtgatgactcagtctccactctccctgcccgtcacccttggacagccggcctccatctcctgcaagtccagtcagagtctgttagacagtggaaatcaaaagaacttcttaacctggtatcagcagaaaccagggaaagctcctaagctcctgatctattgggcatccactagggaatctggggtcccctcgaggttcagtggcagtggatctgggacagatttcacctttaccatcagtagcctggaagctgaagatgctgcaacatattactgtcagaatgattatagttatccgtacacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 82 - protein, 117 aa, Artificial Sequence
  source: /note=“Description of Artificial Sequence: Synthetic polypeptide”
QVQLVQSGAEVKKPGASVKVSCKASGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSS

SEQ ID NO: 83 - DNA, 351 bp, Artificial Sequence
  source: /note=“Description of Artificial Sequence: Synthetic polynucleotide”
caggttcagctggtgcagtctggagctgaggtgaagaagcctggggcctcagtgaaggtctcctgcaaggcttctggctacacattcaccacttactggatgcactggatcaggcagtccccatcgagaggccttgagtggctgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttactggggccagggcaccaccgtgaccgtgtcctcc

SEQ ID NO: 84 - protein, 444 aa, Artificial Sequence
  source: /note=“Description of Artificial Sequence: Synthetic polypeptide”
QVQLVQSGAEVKKPGASVKVSCKASGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - DNA, 1332 bp, Artificial Sequence
  source: /note=“Description of Artificial Sequence: Synthetic polynucleotide”
caggttcagctggtgcagtctggagctgaggtgaagaagcctggggcctcagtgaaggtctcctgcaaggcttctggctacacattcaccacttactggatgcactggatcaggcagtccccatcgagaggccttgagtggctgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttactggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 86 - protein, 117 aa, Artificial Sequence
  source: /note=“Description of Artificial Sequence: Synthetic polypeptide”
EVQLVQSGAEVKKPGESLRISCKGSGYTFTTYWMHWVRQAPGQGLEWMGNIYPGTGGSNFDEKFKNRFTISRDNSKNTLYLQMNSLRAEDTAVYYCTRWTTGTGAYWGQGTTVTVSS

SEQ ID NO: 87 - DNA, 351 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactgggtgcgacaggcccctggacaagggcttgagtggatgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttattggggccagggcaccaccgtgaccgtgtcctcc

SEQ ID NO: 88 - protein, 444 aa, Artificial Sequence
  source: /note=“Description of Artificial Sequence: Synthetic polypeptide”
EVQLVQSGAEVKKPGESLRISCKGSGYTFTTYWMHWVRQAPGQGLEWMGNIYPGTGGSNFDEKFKNRFTISRDNSKNTLYLQMNSLRAEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - DNA, 1332 bp, Artificial Sequence
  source: /note=“Description of Artificial Sequence: Synthetic polynucleotide”
gaagtgcagctggtgcagtctggagcagaggtgaaaaagcccggggagtctctgaggatctcctgtaagggttctggctacacattcaccacttactggatgcactgggtgcgacaggcccctggacaagggcttgagtggatgggtaatatttatcctggtactggtggttctaacttcgatgagaagttcaagaacagattcaccatctccagagacaattccaagaacacgctgtatcttcaaatgaacagcctgagagccgaggacacggccgtgtattactgtacaagatggactactgggacgggagcttattggggccagggcaccaccgtgaccgtgtcctccgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 90 - DNA, 351 bp, Artificial Sequence
  source: /note=“Description of Artificial Sequence: Synthetic polynucleotide”
gaagtgcagctggtgcagtctggcgccgaagtgaagaagcctggcgagtccctgcggatctcctgcaagggctctggctacaccttcaccacctactggatgcactgggtgcgacaggctaccggccagggcctggaatggatgggcaacatctatcctggcaccggcggctccaacttcgacgagaagttcaagaacagagtgaccatcaccgccgacaagtccacctccaccgcctacatggaactgtcctccctgagatccgaggacaccgccgtgtactactgcacccggtggacaaccggcacaggcgcttattggggccagggcaccacagtgaccgtgtcctct

SEQ ID NO: 91 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 92 - DNA, 1329 bp, Artificial Sequence
  source: /note=“Description of Artificial Sequence: Synthetic polynucleotide”
gaagtgcagctggtgcagtctggcgccgaagtgaagaagcctggcgagtccctgcggatctcctgcaagggctctggctacaccttcaccacctactggatgcactgggtgcgacaggctaccggccagggcctggaatggatgggcaacatctatcctggcaccggcggctccaacttcgacgagaagttcaagaacagagtgaccatcaccgccgacaagtccacctccaccgcctacatggaactgtcctccctgagatccgaggacaccgccgtgtactactgcacccggtggacaaccggcacaggcgcttattggggccagggcaccacagtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 93 - DNA, 339 bp, Artificial Sequence
  source: /note=“Description of Artificial Sequence: Synthetic polynucleotide”
gagatcgtgctgacccagtcccctgccaccctgtcactgtctccaggcgagagagctaccctgtcctgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgagtttaccctgaccatctccagcctgcagcccgacgacttcgccacctactactgccagaacgactactcctacccctacaccttcggccagggcaccaaggtggaaatcaag

SEQ ID NO: 94 - DNA, 660 bp, Artificial Sequence
  source: /note=“Description of Artificial Sequence: Synthetic polynucleotide”
gagatcgtgctgacccagtcccctgccaccctgtcactgtctccaggcgagagagctaccctgtcctgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgagtttaccctgaccatctccagcctgcagcccgacgacttcgccacctactactgccagaacgactactcctacccctacaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 95 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 96 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 97 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 98 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 99 - DNA, 339 bp, Artificial Sequence
  source: /note=“Description of Artificial Sequence: Synthetic polynucleotide”
gagatcgtgctgacccagtcccccgacttccagtccgtgacccccaaagaaaaagtgaccatcacatgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgactttaccttcaccatctccagcctggaagccgaggacgccgccacctactactgccagaacgactactcctacccctacaccttcggccagggcaccaaggtggaaatcaag

SEQ ID NO: 100 - DNA, 660 bp, Artificial Sequence
  source: /note=“Description of Artificial Sequence: Synthetic polynucleotide”
gagatcgtgctgacccagtcccccgacttccagtccgtgacccccaaagaaaaagtgaccatcacatgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgactttaccttcaccatctccagcctggaagccgaggacgccgccacctactactgccagaacgactactcctacccctacaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 101 - DNA, 351 bp, Artificial Sequence
  source: /note=“Description of Artificial Sequence: Synthetic polynucleotide”
gaagtgcagctggtgcagtctggcgccgaagtgaagaagcctggcgagtccctgcggatctcctgcaagggctctggctacaccttcaccacctactggatgcactggatccggcagtccccctctaggggcctggaatggctgggcaacatctaccctggcaccggcggctccaacttcgacgagaagttcaagaacaggttcaccatctcccgggacaactccaagaacaccctgtacctgcagatgaactccctgcgggccgaggacaccgccgtgtactactgtaccagatggaccaccggaaccggcgcctattggggccagggcacaacagtgaccgtgtcctcc

SEQ ID NO: 102 - protein, 443 aa, Artificial Sequence
  source: /note=“Description of Artificial Sequence: Synthetic polypeptide”
EVQLVQSGAEVKKPGESLRISCKGSGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 103 - DNA, 1329 bp, Artificial Sequence
  source: /note=“Description of Artificial Sequence: Synthetic polynucleotide”
gaagtgcagctggtgcagtctggcgccgaagtgaagaagcctggcgagtccctgcggatctcctgcaagggctctggctacaccttcaccacctactggatgcactggatccggcagtccccctctaggggcctggaatggctgggcaacatctaccctggcaccggcggctccaacttcgacgagaagttcaagaacaggttcaccatctcccgggacaactccaagaacaccctgtacctgcagatgaactccctgcgggccgaggacaccgccgtgtactactgtaccagatggaccaccggaaccggcgcctattggggccagggcacaacagtgaccgtgtcctccgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 104 - DNA, 339 bp, Artificial Sequence
  source: /note=“Description of Artificial Sequence: Synthetic polynucleotide”
gagatcgtgctgacccagtcccctgccaccctgtcactgtctccaggcgagagagctaccctgtcctgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgactttaccttcaccatctccagcctggaagccgaggacgccgccacctactactgccagaacgactactcctacccctacaccttcggccagggcaccaaggtggaaatcaag

SEQ ID NO: 105 - DNA, 660 bp, Artificial Sequence
  source: /note=“Description of Artificial Sequence: Synthetic polynucleotide”
gagatcgtgctgacccagtcccctgccaccctgtcactgtctccaggcgagagagctaccctgtcctgcaagtcctcccagtccctgctggactccggcaaccagaagaacttcctgacctggtatcagcagaagcccggccaggcccccagactgctgatctactgggcctccacccgggaatctggcgtgccctctagattctccggctccggctctggcaccgactttaccttcaccatctccagcctggaagccgaggacgccgccacctactactgccagaacgactactcctacccctacaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 106 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 107 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 108 - DNA, 15 bp, Artificial Sequence
  source: /note=“Description of Artificial Sequence: Synthetic oligonucleotide”
acttactggatgcac

SEQ ID NO: 109 - DNA, 51 bp, Artificial Sequence
  source: /note=“Description of Artificial Sequence: Synthetic oligonucleotide”
aatatttatcctggtactggtggttctaacttcgatgagaagttcaagaac

SEQ ID NO: 110 - DNA, 24 bp, Artificial Sequence
  source: /note=“Description of Artificial Sequence: Synthetic oligonucleotide”
tggactactgggacgggagcttat

SEQ ID NO: 111 - DNA, 21 bp, Artificial Sequence
  source: /note=“Description of Artificial Sequence: Synthetic oligonucleotide”
ggctacacattcaccacttac

SEQ ID NO: 112 - DNA, 18 bp, Artificial Sequence
  source: /note=“Description of Artificial Sequence: Synthetic oligonucleotide”
tatcctggtactggtggt

SEQ ID NO: 113 - DNA, 51 bp, Artificial Sequence
  source: /note=“Description of Artificial Sequence: Synthetic oligonucleotide”
aagtccagtcagagtctgttagacagtggaaatcaaaagaacttcttgacc

SEQ ID NO: 114 - DNA, 21 bp, Artificial Sequence
  source: /note=“Description of Artificial Sequence: Synthetic oligonucleotide”
tgggcatccactagggaatct

SEQ ID NO: 115 - DNA, 27 bp, Artificial Sequence
  source: /note=“Description of Artificial Sequence: Synthetic oligonucleotide”
cagaatgattatagttatccgtgcacg

SEQ ID NO: 116 - DNA, 39 bp, Artificial Sequence
  source: /note=“Description of Artificial Sequence: Synthetic oligonucleotide”
agtcagagtctgttagacagtggaaatcaaaagaacttc

SEQ ID NO: 117 - DNA, 9 bp, Artificial Sequence
  source: /note=“Description of Artificial Sequence: Synthetic oligonucleotide”
tgggcatcc

SEQ ID NO: 118 - DNA, 18 bp, Artificial Sequence
  source: /note=“Description of Artificial Sequence: Synthetic oligonucleotide”
gattatagttatccgtgc

SEQ ID NO: 119 - DNA, 27 bp, Artificial Sequence
  source: /note=“Description of Artificial Sequence: Synthetic oligonucleotide”
cagaatgattatagttatccgtacacg

SEQ ID NO: 120 - DNA, 18 bp, Artificial Sequence
  source: /note=“Description of Artificial Sequence: Synthetic oligonucleotide”
gattatagttatccgtac

SEQ ID NO: 121 - DNA, 51 bp, Artificial Sequence
  source: /note=“Description of Artificial Sequence: Synthetic oligonucleotide”
aagtccagtcagagtctgttagacagtggaaatcaaaagaacttcttaacc

SEQ ID NO: 122 - DNA, 15 bp, Artificial Sequence
  source: /note=“Description of Artificial Sequence: Synthetic oligonucleotide”
acctactggatgcac

SEQ ID NO: 123 - DNA, 51 bp, Artificial Sequence
  source: /note=“Description of Artificial Sequence: Synthetic oligonucleotide”
aacatctatcctggcaccggcggctccaacttcgacgagaagttcaagaac

SEQ ID NO: 124 - DNA, 24 bp, Artificial Sequence
  source: /note=“Description of Artificial Sequence: Synthetic oligonucleotide”
tggacaaccggcacaggcgcttat

SEQ ID NO: 125 - DNA, 21 bp, Artificial Sequence
  source: /note=“Description of Artificial Sequence: Synthetic oligonucleotide”
ggctacaccttcaccacctac

SEQ ID NO: 126 - DNA, 18 bp, Artificial Sequence
  source: /note=“Description of Artificial Sequence: Synthetic oligonucleotide”
tatcctggcaccggcggc

SEQ ID NO: 127 - DNA, 51 bp, Artificial Sequence
  source: /note=“Description of Artificial Sequence: Synthetic oligonucleotide”
aagtcctcccagtccctgctggactccggcaaccagaagaacttcctgacc

SEQ ID NO: 128 - DNA, 21 bp, Artificial Sequence
  source: /note=“Description of Artificial Sequence: Synthetic oligonucleotide”
tgggcctccacccgggaatct

SEQ ID NO: 129 - DNA, 27 bp, Artificial Sequence
  source: /note=“Description of Artificial Sequence: Synthetic oligonucleotide”
cagaacgactactcctacccctacacc

SEQ ID NO: 130 - DNA, 39 bp, Artificial Sequence
  source: /note=“Description of Artificial Sequence: Synthetic oligonucleotide”
tcccagtccctgctggactccggcaaccagaagaacttc

SEQ ID NO: 131 - DNA, 9 bp, Artificial Sequence
  source: /note=“Description of Artificial Sequence: Synthetic oligonucleotide”
tgggcctcc

SEQ ID NO: 132 - DNA, 18 bp, Artificial Sequence
  source: /note=“Description of Artificial Sequence: Synthetic oligonucleotide”
gactactcctacccctac

SEQ ID NO: 133 - DNA, 15 bp, Artificial Sequence
  source: /note=“Description of Artificial Sequence: Synthetic oligonucleotide”
acctactggatgcac

SEQ ID NO: 134 - DNA, 51 bp, Artificial Sequence
  source: /note=“Description of Artificial Sequence: Synthetic oligonucleotide”
aatatctaccccggcaccggcggctctaacttcgacgagaagtttaagaat

SEQ ID NO: 135 - DNA, 24 bp, Artificial Sequence
  source: /note=“Description of Artificial Sequence: Synthetic oligonucleotide”
tggactaccggcacaggcgcctac

SEQ ID NO: 136 - DNA, 21 bp, Artificial Sequence
  source: /note=“Description of Artificial Sequence: Synthetic oligonucleotide”
ggctacaccttcactacctac

SEQ ID NO: 137 - DNA, 18 bp, Artificial Sequence
  source: /note=“Description of Artificial Sequence: Synthetic oligonucleotide”
taccccggcaccggcggc

SEQ ID NO: 138 - DNA, 51 bp, Artificial Sequence
  source: /note=“Description of Artificial Sequence: Synthetic oligonucleotide”
aaatctagtcagtcactgctggatagcggtaatcagaagaacttcctgacc

SEQ ID NO: 139 - DNA, 21 bp, Artificial Sequence
  source: /note=“Description of Artificial Sequence: Synthetic oligonucleotide”
tgggcctctactagagaatca

SEQ ID NO: 140 - DNA, 27 bp, Artificial Sequence
  source: /note=“Description of Artificial Sequence: Synthetic oligonucleotide”
cagaacgactatagctacccctacacc

SEQ ID NO: 141 - DNA, 39 bp, Artificial Sequence
  source: /note=“Description of Artificial Sequence: Synthetic oligonucleotide”
agtcagtcactgctggatagcggtaatcagaagaacttc

SEQ ID NO: 142 - DNA, 9 bp, Artificial Sequence
  source: /note=“Description of Artificial Sequence: Synthetic oligonucleotide”
tgggcctct

SEQ ID NO: 143 - DNA, 18 bp, Artificial Sequence
  source: /note=“Description of Artificial Sequence: Synthetic oligonucleotide”
gactatagctacccctac

SEQ ID NO: 144 - DNA, 51 bp, Artificial Sequence
  source: /note=“Description of Artificial Sequence: Synthetic oligonucleotide”
aacatctaccctggcaccggcggctccaacttcgacgagaagttcaagaac

SEQ ID NO: 145 - DNA, 24 bp, Artificial Sequence
  source: /note=“Description of Artificial Sequence: Synthetic oligonucleotide”
tggaccaccggaaccggcgcctat

SEQ ID NO: 146 - DNA, 18 bp, Artificial Sequence
  source: /note=“Description of Artificial Sequence: Synthetic oligonucleotide”
taccctggcaccggcggc

SEQ ID NO: 147 - protein, 25 aa, Artificial Sequence
  source: /note=“Description of Artificial Sequence: Synthetic peptide”
EVQLVQSGAEVKKPGESLRISCKGS

SEQ ID NO: 148 - DNA, 75 bp, Artificial Sequence
  source: /note=“Description of Artificial Sequence: Synthetic oligonucleotide”
gaagtgcagctggtgcagtctggagcagaggtgaaaaagcccggggagtctctgaggatctcctgtaagggttct

SEQ ID NO: 149 - DNA, 75 bp, Artificial Sequence
  source: /note=“Description of Artificial Sequence: Synthetic oligonucleotide”
gaagtgcagctggtgcagtctggcgccgaagtgaagaagcctggcgagtccctgcggatctcctgcaagggctct

SEQ ID NO: 150 - DNA, 75 bp, Artificial Sequence
  source: /note=“Description of Artificial Sequence: Synthetic oligonucleotide”
gaggtgcagctggtgcagtcaggcgccgaagtgaagaagcccggcgagtcactgagaattagctgtaaaggttca

SEQ ID NO: 151 - protein, 25 aa, Artificial Sequence [sequence omitted]

SEQ ID NO: 152 - DNA, 75 bp, Artificial Sequence
  source: /note=“Description of Artificial Sequence: Synthetic oligonucleotide”
caggttcagctggtgcagtctggagctgaggtgaagaagcctggggcctcagtgaaggtctcctgcaaggcttct

SEQ ID NO: 153 - protein, 14 aa, Artificial Sequence
  source: /note=“Description of Artificial Sequence: Synthetic peptide”
WVRQATGQGLEWMG

SEQ ID NO: 154 - DNA, 42 bp, Artificial Sequence
  source: /note=“Description of Artificial Sequence: Synthetic oligonucleotide”
tgggtgcgacaggccactggacaagggcttgagtggatgggt

SEQ ID NO: 155 - DNA, 42 bp, Artificial Sequence
  source: /note=“Description of Artificial Sequence: Synthetic oligonucleotide”
tgggtgcgacaggctaccggccagggcctggaatggatgggc

SEQ ID NO: 156 - DNA, 42 bp, Artificial Sequence
  source: /note=“Description of Artificial Sequence: Synthetic oligonucleotide”
tgggtccgccaggctaccggtcaaggcctcgagtggatgggt

SEQ ID NO: 157 - protein, 14 aa, Artificial Sequence
  source: /note=“Description of Artificial Sequence: Synthetic peptide”
WIRQSPSRGLEWLG

SEQ ID NO: 158 - DNA, 42 bp, Artificial Sequence
  source: /note=“Description of Artificial Sequence: Synthetic oligonucleotide”
tggatcaggcagtccccatcgagaggccttgagtggctgggt

SEQ ID NO: 159 - DNA, 42 bp, Artificial Sequence
  source: /note=“Description of Artificial Sequence: Synthetic oligonucleotide”
tggatccggcagtccccctctaggggcctggaatggctgggc

SEQ ID NO: 160 - protein, 14 aa, Artificial Sequence
  source: /note=“Description of Artificial Sequence: Synthetic peptide”
WVRQAPGQGLEWMG

SEQ ID NO: 161 - DNA, 42 bp, Artificial Sequence
  source: /note=“Description of Artificial Sequence: Synthetic oligonucleotide”
tgggtgcgacaggcccctggacaagggcttgagtggatgggt

SEQ ID NO: 162 - protein, 32 aa, Artificial Sequence
  source: /note=“Description of Artificial Sequence: Synthetic polypeptide”
RVTITADKSTSTAYMELSSLRSEDTAVYYCTR

SEQ ID NO: 163 - DNA, 96 bp, Artificial Sequence
  source: /note=“Description of Artificial Sequence: Synthetic oligonucleotide”
agagtcacgattaccgcggacaaatccacgagcacagcctacatggagctgagcagcctgagatctgaggacacggccgtgtattactgtacaaga

SEQ ID NO: 164 - DNA, 96 bp, Artificial Sequence
  source: /note=“Description of Artificial Sequence: Synthetic oligonucleotide”
agagtgaccatcaccgccgacaagtccacctccaccgcctacatggaactgtcctccctgagatccgaggacaccgccgtgtactactgcacccgg

SEQ ID NO: 165 - DNA, 96 bp, Artificial Sequence
  source: /note=“Description of Artificial Sequence: Synthetic oligonucleotide”
agagtgactatcaccgccgataagtctactagcaccgcctatatggaactgtctagcctgagatcagaggacaccgccgtctactactgcactagg

SEQ ID NO: 166 - protein, 32 aa, Artificial Sequence
  source: /note=“Description of Artificial Sequence: Synthetic polypeptide”
RFTISRDNSKNTLYLQMNSLRAEDTAVYYCTR

SEQ ID NO: 167 - DNA, 96 bp, Artificial Sequence
  source: /note=“Description of Artificial Sequence: Synthetic oligonucleotide”
agattcaccatctccagagacaattccaagaacacgctgtatcttcaaatgaacagcctgagagccgaggacacggccgtgtattactgtacaaga

SEQ ID NO: 168 - DNA, 96 bp, Artificial Sequence
  source: /note=“Description of Artificial Sequence: Synthetic oligonucleotide”
aggttcaccatctcccgggacaactccaagaacaccctgtacctgcagatgaactccctgcgggccgaggacaccgccgtgtactactgtaccaga

SEQ ID NO: 169 - protein, 11 aa, Artificial Sequence
  source: /note=“Description of Artificial Sequence: Synthetic peptide”
WGQGTTVTVSS

SEQ ID NO: 170 - DNA, 33 bp, Artificial Sequence
  source: /note=“Description of Artificial Sequence: Synthetic oligonucleotide”
tggggccagggcaccaccgtgaccgtgtcctcc

SEQ ID NO: 171 - DNA, 33 bp, Artificial Sequence
  source: /note=“Description of Artificial Sequence: Synthetic oligonucleotide”
tggggccagggcaccacagtgaccgtgtcctct

SEQ ID NO: 172 - DNA, 33 bp, Artificial Sequence
  source: /note=“Description of Artificial Sequence: Synthetic oligonucleotide”
tggggtcaaggcactaccgtgaccgtgtctagc

SEQ ID NO: 173 - DNA, 33 bp, Artificial Sequence
  source: /note=“Description of Artificial Sequence: Synthetic oligonucleotide”
tggggccagggcacaacagtgaccgtgtcctcc

SEQ ID NO: 174 - protein, 23 aa, Artificial Sequence
  source: /note=“Description of Artificial Sequence: Synthetic peptide”
EIVLTQSPDFQSVTPKEKVTITC

SEQ ID NO: 175 - DNA, 69 bp, Artificial Sequence
  source: /note=“Description of Artificial Sequence: Synthetic oligonucleotide”
gaaattgtgctgactcagtctccagactttcagtctgtgactccaaaggagaaagtcaccatcacctgc

SEQ ID NO: 176 - DNA, 69 bp, Artificial Sequence
  source: /note=“Description of Artificial Sequence: Synthetic oligonucleotide”
gagatcgtgctgacccagtcccccgacttccagtccgtgacccccaaagaaaaagtgaccatcacatgc

SEQ ID NO: 177 - protein, 23 aa, Artificial Sequence
  source: /note=“Description of Artificial Sequence: Synthetic peptide”
EIVLTQSPATLSLSPGERATLSC

SEQ ID NO: 178 - DNA, 69 bp, Artificial Sequence
  source: /note=“Description of Artificial Sequence: Synthetic oligonucleotide”
gaaattgtgttgacacagtctccagccaccctgtctttgtctccaggggaaagagccaccctctcctgc

SEQ ID NO: 179 - DNA, 69 bp, Artificial Sequence
  source: /note=“Description of Artificial Sequence: Synthetic oligonucleotide”
gagatcgtgctgacccagtcccctgccaccctgtcactgtctccaggcgagagagctaccctgtcctgc

SEQ ID NO: 180 - DNA, 69 bp, Artificial Sequence
  source: /note=“Description of Artificial Sequence: Synthetic oligonucleotide”
gagatcgtcctgactcagtcacccgctaccctgagcctgagccctggcgagcgggctacactgagctgt

SEQ ID NO: 181 - protein, 23 aa, Artificial Sequence
  source: /note=“Description of Artificial Sequence: Synthetic peptide”
DIVMTQTPLSLPVTPGEPASISC

SEQ ID NO: 182 - DNA, 69 bp, Artificial Sequence
  source: /note=“Description of Artificial Sequence: Synthetic oligonucleotide”
gatattgtgatgacccagactccactctccctgcccgtcacccctggagagccggcctccatctcctgc

SEQ ID NO: 183 - protein, 23 aa, Artificial Sequence
  source: /note=“Description of Artificial Sequence: Synthetic peptide”
DVVMTQSPLSLPVTLGQPASISC

SEQ ID NO: 184 - DNA, 69 bp, Artificial Sequence
  source: /note=“Description of Artificial Sequence: Synthetic oligonucleotide”
gatgttgtgatgactcagtctccactctccctgcccgtcacccttggacagccggcctccatctcctgc

SEQ ID NO: 185 - protein, 23 aa, Artificial Sequence
  source: /note=“Description of Artificial Sequence: Synthetic peptide”
DIQMTQSPSSLSASVGDRVTITC

SEQ ID NO: 186 - DNA, 69 bp, Artificial Sequence
  source: /note=“Description of Artificial Sequence: Synthetic oligonucleotide”
gacatccagatgacccagtctccatcctccctgtctgcatctgtaggagacagagtcaccatcacttgc

SEQ ID NO: 187 - protein, 15 aa, Artificial Sequence
  source: /note=“Description of Artificial Sequence: Synthetic peptide”
WYQQKPGQAPRLLIY

SEQ ID NO: 188 - DNA, 45 bp, Artificial Sequence
  source: /note=“Description of Artificial Sequence: Synthetic oligonucleotide”
tggtaccagcagaaacctggccaggctcccaggctcctcatctat

SEQ ID NO: 189 - DNA, 45 bp, Artificial Sequence
  source: /note=“Description of Artificial Sequence: Synthetic oligonucleotide”
tggtatcagcagaagcccggccaggcccccagactgctgatctac

SEQ ID NO: 190 - DNA, 45 bp, Artificial Sequence
  source: /note=“Description of Artificial Sequence: Synthetic oligonucleotide”
tggtatcagcagaagcccggtcaagcccctagactgctgatctac

SEQ ID NO: 191 - protein, 15 aa, Artificial Sequence
  source: /note=“Description of Artificial Sequence: Synthetic peptide”
WYQQKPGKAPKLLIY

SEQ ID NO: 192 - DNA, 45 bp, Artificial Sequence
  source: /note=“Description of Artificial Sequence: Synthetic oligonucleotide”
tggtatcagcagaaaccagggaaagctcctaagctcctgatctat

SEQ ID NO: 193 - DNA, 45 bp, Artificial Sequence
  source: /note=“Description of Artificial Sequence: Synthetic oligonucleotide”
tggtatcagcagaagcccggtaaagcccctaagctgctgatctac

SEQ ID NO: 194 - protein, 15 aa, Artificial Sequence
  source: /note=“Description of Artificial Sequence: Synthetic peptide”
WYLQKPGQSPQLLIY

SEQ ID NO: 195 - DNA, 45 bp, Artificial Sequence
  source: /note=“Description of Artificial Sequence: Synthetic oligonucleotide”
tggtacctgcagaagccagggcagtctccacagctcctgatctat

SEQ ID NO: 196 - protein, 32 aa, Artificial Sequence
  source: /note=“Description of Artificial Sequence: Synthetic polypeptide”
GVPSRFSGSGSGTDFTFTISSLEAEDAATYYC

SEQ ID NO: 197 - DNA, 96 bp, Artificial Sequence
  source: /note=“Description of Artificial Sequence: Synthetic oligonucleotide”
ggggtcccctcgaggttcagtggcagtggatctgggacagatttcacctttaccatcagtagcctggaagctgaagatgctgcaacatattactgt

SEQ ID NO: 198 - DNA, 96 bp, Artificial Sequence
  source: /note=“Description of Artificial Sequence: Synthetic oligonucleotide”
ggcgtgccctctagattctccggctccggctctggcaccgactttaccttcaccatctccagcctggaagccgaggacgccgccacctactactgc

SEQ ID NO: 199 - DNA, 96 bp, Artificial Sequence
  source: /note=“Description of Artificial Sequence: Synthetic oligonucleotide”
ggcgtgccctctaggtttagcggtagcggtagtggcaccgacttcaccttcactatctctagcctggaagccgaggacgccgctacctactactgt

SEQ ID NO: 200 - protein, 32 aa, Artificial Sequence
  source: /note=“Description of Artificial Sequence: Synthetic polypeptide”
GIPPRFSGSGYGTDFTLTINNIESEDAAYYFC

SEQ ID NO: 201 - DNA, 96 bp, Artificial Sequence
  source: /note=“Description of Artificial Sequence: Synthetic oligonucleotide”
gggatcccacctcgattcagtggcagcgggtatggaacagattttaccctcacaattaataacatagaatctgaggatgctgcatattacttctgt

SEQ ID NO: 202 - protein, 32 aa, Artificial Sequence
  source: /note=“Description of Artificial Sequence: Synthetic polypeptide”
GVPSRFSGSGSGTEFTLTISSLQPDDFATYYC

SEQ ID NO: 203 - DNA, 96 bp, Artificial Sequence
  source: /note=“Description of Artificial Sequence: Synthetic oligonucleotide”
ggggtcccatcaaggttcagcggcagtggatctgggacagaattcactctcaccatcagcagcctgcagcctgatgattttgcaacttattactgt

SEQ ID NO: 204 - DNA, 96 bp, Artificial Sequence
  source: /note=“Description of Artificial Sequence: Synthetic oligonucleotide”
ggcgtgccctctagattctccggctccggctctggcaccgagtttaccctgaccatctccagcctgcagcccgacgacttcgccacctactactgc

SEQ ID NO: 205 - protein, 32 aa, Artificial Sequence
  source: /note=“Description of Artificial Sequence: Synthetic polypeptide”
GVPSRFSGSGSGTDFTFTISSLQPEDIATYYC

SEQ ID NO: 206 - DNA, 96 bp, Artificial Sequence
  source: /note=“Description of Artificial Sequence: Synthetic oligonucleotide”
ggggtcccatcaaggttcagtggaagtggatctgggacagattttactttcaccatcagcagcctgcagcctgaagatattgcaacatattactgt

SEQ ID NO: 207 - DNA, 96 bp, Artificial Sequence
  source: /note=“Description of Artificial Sequence: Synthetic oligonucleotide”
ggcgtgccctctaggtttagcggtagcggtagtggcaccgacttcaccttcactatctctagcctgcagcccgaggatatcgctacctactactgt

SEQ ID NO: 208 - protein, 10 aa, Artificial Sequence
  source: /note=“Description of Artificial Sequence: Synthetic peptide”
FGQGTKVEIK

SEQ ID NO: 209 - DNA, 30 bp, Artificial Sequence
  source: /note=“Description of Artificial Sequence: Synthetic oligonucleotide”
ttcggccaagggaccaaggtggaaatcaaa

SEQ ID NO: 210 - DNA, 30 bp, Artificial Sequence
  source: /note=“Description of Artificial Sequence: Synthetic oligonucleotide”
ttcggccagggcaccaaggtggaaatcaag

SEQ ID NO: 211 - DNA, 30 bp, Artificial Sequence
  source: /note=“Description of Artificial Sequence: Synthetic oligonucleotide”
ttcggtcaaggcactaaggtcgagattaag

SEQ ID NO: 212 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 213 - protein, 107 aa, Homo sapiens [sequence omitted]

SEQ ID NO: 214 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 215 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 19 aa, Artificial Sequence
  source: /note=“Description of Artificial Sequence: Synthetic peptide”
MEWSWVFLFFLSVTTGVHS

SEQ ID NO: 220 - protein, 20 aa, Artificial Sequence
  source: /note=“Description of Artificial Sequence: Synthetic peptide”
MSVPTQVLGLLLLWLTDARC

SEQ ID NO: 221 - protein, 19 aa, Artificial Sequence
  source: /note=“Description of Artificial Sequence: Synthetic peptide”
MAWVWTLPFLMAAAQSVQA

SEQ ID NO: 222 - protein, 20 aa, Artificial Sequence
  source: /note=“Description of Artificial Sequence: Synthetic peptide”
MSVLTQVLALLLLWLTGTRC

SEQ ID NO: 223 - DNA, 24 bp, Artificial Sequence
  source: /note=“Description of Artificial Sequence: Synthetic oligonucleotide”
tggactactgggacgggagcttac

SEQ ID NO: 224 - protein, 10 aa, Artificial Sequence
  source: /note=“Description of Artificial Sequence: Synthetic peptide”
GYTFTTYWMH

SEQ ID NO: 225-227 - skipped (3 entries)

SEQ ID NO: 228 - protein, 134 aa, Artificial Sequence
  source: /note=“Description of Artificial Sequence: Synthetic polypeptide”
QVQLQQPGSELVRPGASVKLSCKASGYTFTTYWMHWVRQRPGQGLEWIGNIYPGTGGSNFDEKFKNRTSLTVDTSSTTAYMHLASLTSEDSAVYYCTRWTTGTGAYWGQGTLVTVSAAKTTPPSVYPLAPGSAA

SEQ ID NO: 229 - protein, 116 aa, Artificial Sequence
  source: /note=“Description of Artificial Sequence: Synthetic polypeptide”
DIVMTQSPSSLTVTAGEKVTMSCKSSQSLLDSGNQKNFLTWYQQKPGQPPKLLIFWASTRESGVPDRFTGSGSVTDFTLTISSVQAEDLAVYYCQNDYSYPCTFGGGTKLEIKRAD

SEQ ID NO: 230 - protein, 98 aa, Artificial Sequence
  source: /note=“Description of Artificial Sequence: Synthetic polypeptide”
QVQLQQPGSELVRPGASVKLSCKASGYTFTSYWMHWVKQRHGQGLEWIGNIYPGSGSTNYDEKFKSKGTLTVDTSSSTAYMHLSSLTSEDSAVYYCTR

SEQ ID NO: 231 - protein, 101 aa, Artificial Sequence
  source: /note=“Description of Artificial Sequence: Synthetic polypeptide”
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYP

SEQ ID NO: 232 - DNA, 37 bp, Artificial Sequence
  source: /note=“Description of Artificial Sequence: Synthetic oligonucleotide”
  CDS (2)..(37)
gtgcacgttcggaggggggaccaagctggaaataaaa

SEQ ID NO: 233 - protein, 12 aa, Artificial Sequence
  source: /note=“Description of Artificial Sequence: Synthetic peptide”
CTFGGGTKLEIK

SEQ ID NO: 234 - DNA, 38 bp, Artificial Sequence
  source: /note=“Description of Artificial Sequence: Synthetic oligonucleotide”
  CDS (2)..(37)
gtacacgttcggaggggggaccaagctggaaataaaac

SEQ ID NO: 235 - protein, 12 aa, Artificial Sequence
  source: /note=“Description of Artificial Sequence: Synthetic peptide”
YTFGGGTKLEIK

(19) United States
(12) Patent Application Publication
(10) Pub. No.: US 2021/0363254 A1
(43) Pub. Date: Nov. 25, 2021

(54) PHARMACEUTICAL COMBINATIONS

(71) Applicant: Novartis AG, Basel (CH)

(72) Inventors: Stephane FERRETTI, Huningue (FR); Nelson GUERREIRO, Basel (CH); Ensar HALILOVIC, Canton, MA (US); Sebastien JEAY, Niffer (FR); Astrid JULLION, Sierentz (FR); Jinsheng LIANG, Shrewsbury, MA (US); Christophe MEILLE, Sierentz (FR); Hui-Qin WANG, Lexington, MA (US); Jens WUERTHNER, Steinen (DE)

(21) Appl. No.: 16/981,828

(22) PCT Filed: Mar. 18, 2019
(86) PCT No.: PCT/IB2019/052166

§ 371 Date: Sep. 17, 2020

Related U.S. Application Data
(60) Provisional application No. 62/645,754, filed on Mar. 20, 2018.

(51) Int. Cl. C07K 16/28; A61P 35/00; C07D 487/04

(52) U.S. Cl. CPC C07K 16/2818; A61K 2039/505; C07D 487/04; A61P 35/00

(57) ABSTRACT

The present invention relates to a pharmaceutical combination which comprises (a) at least one antibody molecule (e.g., humanized antibody molecules) that bind to Programmed Death 1 (PD-1), and (b) a HDM2-p53 interaction inhibitor, said combination for simultaneous, separate or sequential administration for use in the treatment of a proliferative disease, a pharmaceutical composition comprising such combination; a method of treating a subject having a proliferative disease comprising administration of said combination to a subject in need thereof; use of such combination for the treatment of proliferative disease; and a commercial package comprising such combination; said proliferative disease being a TP53 wildtype tumor, in particular TP53 wildtype renal cell carcinoma (RCC) or TP53 wildtype colorectal cancer (CRC).

### SEQUENCE LISTING

The instant application contains a Sequence Listing which has been submitted electronically in ASCII format and is hereby incorporated by reference in its entirety. Said ASCII copy is named PAT058095_SL.TXT and is 190,381 bytes in size.

### FIELD OF THE INVENTION

The present invention relates to a pharmaceutical combination which comprises (a) at least one antibody molecule (e.g., humanized antibody molecules) that bind to Programmed Death 1 (PD-1), also referred herein as “PD-1 inhibitor”, and (b) a HDM2-p53 interaction inhibitor, also referred herein as “HMD2 inhibitor”, said combination for simultaneous, separate or sequential administration for use in the treatment of a proliferative disease, a pharmaceutical composition comprising such combination; a method of treating a subject having a proliferative disease comprising administration of said combination to a subject in need thereof; use of such combination for the treatment of proliferative disease; and a commercial package comprising such combination; said proliferative disease being a tumor, in particular a TP53 wildtype tumor, in particular a TP53 wildtype solid tumor, in particular TP53 wildtype renal cell carcinoma (RCC) or colorectal cancer (CRC).

### BACKGROUND

p53 is induced and activated by a number of potentially tumorigenic processes—including aberrant growth signals, DNA damage, ultraviolet light, and protein kinase inhibitors (Millard M, et al. Curr Pharm Design 2011; 17:536-559)—and regulates genes controlling cell growth arrest, DNA repair, apoptosis, and angiogenesis (Bullock A N & Fersht A R. Nat Rev Cancer 2001; 1:68-76; Vogelstein B, et al. Nature Education 2010; 3(9):6).

Human Double Minute-2 (HDM2) is one of the most important regulators of p53. It binds directly to p53, inhibiting its transactivation, and subsequently directing it towards cytoplasmic degradation (Zhang Y, et al. Nucleic Acids Res 2010; 38:6544-6554).

p53 is one of the most frequently inactivated proteins in human cancer, either through direct mutation of the TP53 gene (found in approximately 50% of all human cancers) (Vogelstein, B et al. Nature 2000; 408:307-310) or via suppressive mechanisms such as overexpression of HDM2 (Zhao Y, et al. BioDiscovery 2013; 8:4).

Potent and selective inhibitors of the HDM2-p53 interaction (also referred to as HDM2 inhibitors or MDM2 inhibitors), e.g. NVP-HDM201, have been shown to restore p53 function in preclinical cell and in vivo models (Holzer P, et al. Poster presented at AACR 2016, Abstract #4855).

The ability of T cells to mediate an immune response against an antigen requires two distinct signaling interactions (Viglietta, V. et al. (2007) Neurotherapeutics 4:666-675; Korman, A. J. et al. (2007) Adv. Immunol. 90:297-339). First, an antigen that has been arrayed on the surface of antigen-presenting cells (APC) is presented to an antigen-specific naive CD4⁺ T cell. Such presentation delivers a signal via the T cell receptor (TCR) that directs the T cell to initiate an immune response specific to the presented antigen. Second, various co-stimulatory and inhibitory signals mediated through interactions between the APC and distinct T cell surface molecules trigger the activation and proliferation of the T cells and ultimately their inhibition.

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8). Other members of the CD28 family include CD28, CTLA-4, ICOS and BTLA. It is one of the target sites in the immune checkpoint pathways that many tumors use to evade attack by the immune system. PD-1 is suggested to exist as a monomer, lacking the unpaired cysteine residue characteristic of other CD28 family members. PD-1 is expressed on activated B cells, T cells, and monocytes.

Given the importance of immune checkpoint pathways in regulating an immune response to tumors, the need exists for developing novel combination therapies that modulate the activity of immunoinhibitory proteins, such as PD-1, thus leading to activation of the immune system. Such agents can be used, e.g., for cancer immunotherapy and treatment of other conditions.

Colorectal cancer (CRC) is the third most common cancer in the world, with approximately 1.4 million people diagnosed in 2012, and the fourth most common cause of death from cancer, with 694,000 deaths (World Cancer Report 2014). Outcomes for patients with CRC are linked to the immune infiltrate in tumors, suggesting CRC may benefit from therapies that stimulate an immune response (Fridman W H, Galon J, Pages F, et al. (2011) Prognostic and predictive impact of intra- and peritumoral immune infiltrates. Cancer Res. p. 5601-5). However, preliminary experience with checkpoint inhibitors of CTLA-4 or PD-1 have been disappointing outside of the mismatch repair-deficient population (Le D T, Uram J N, Wang H, et al. (2015) PD-1 Blockade in Tumors with Mismatch-Repair Deficiency. N. Engl. J. Med. p. 2509-20; and other references Ribas et al. 2005; Chung et al. 2010; Brahmer et al. 2010; Topalian et al. 2012; Brahmer et al. 2012). The reason(s) for lack of efficacy are unclear (Kroemer G, Galluzzi L, Laurence Zitvogel L, et al. (2015) Colorectal cancer: the first neoplasia found to be under immunosurveillance and the last one to respond to immunotherapy? Oncolmmunology 4:7, e1058597-1-3).

Renal cell carcinoma (RCC) is the 16th leading cause of neoplasm-related death worldwide, with 143,000 deaths worldwide in 2012 (Ferlay et al 2015). In the US, there are expected to be >62,000 new cases, and >14,000 deaths from renal cancer in 2016 (Siegel et al 2016). Nivolumab is approved for use in RCC (drug labels for Opdivo® (2014)). Nivolumab has shown a 25 months' median OS in RCC patients beyond first-line therapy compared with everolimus, with a benefit of 5.4 months for patients receiving nivolumab (Mazza C, Escudier B, Albiges L. (2017) Nivolumab in renal cell carcinoma: latest evidence and clinical potential. Ther Adv Med Oncol. p. 171-181). To date, at least 31 studies have investigated the expression of TP53 in RCC. In a meta-analysis of 2519 RCC tumors, the TP53 positive frequency was 24.5% (Noon A P, Vlatkovic N, Polanski R, et. al (2010) p53 and MDM2 in renal cell carcinoma: biomarkers for disease progression and future therapeutic targets?Cancer. p. 116:780-90).

Immunotherapies currently in development have started to offer significant benefit to melanoma cancer patients, including those for whom conventional treatments are ineffective. Recently, pembrolizumab and nivolumab, two inhibitors of the PD-1/PD-L1 interaction have been approved for use in NSCLC and melanoma under the trade names Keytruda® and Opdivo®, respectively.

While inhibitors of the PD-1/PD-L1 interaction are well tolerated and have demonstrated some activity across a remarkable range of cancer types, there remains a needs to complement the therapy with other therapeutic agents to increase the response rate and durability of treatment.

Different dosing regimens were described for HDM2 inhibitors and tested in clinical studies.

E.g. US2013/0245089 discloses a method of treating a patient suffering from cancer by administering to the patient 4-{[(2R,3S,4R,5S)-4-(4-Chloro-2-fluoro-phenyl)-3-(3-chloro-2-fluoro-phenyl)-4-cyano-5-(2, 2-dimethyl-propyl)-pyrrolidine-2-carbonyl]-amino}-3-methoxy-benzoic acid in an amount of from about 800 to about 3000 mg/day for an administration period of up to about 7 days, on days 1-7, of a 28 day treatment cycle, followed by a rest period of from about 21 to about 23 days.

A paper in Clinical Cancer Research by B. Higgins et al. (May 2014) disclosed a 28 days cycle schedule, where RG7388 is administered once weekly three times followed by 13 days of rest (28 days cycle schedule), or where the drug is administered for 5 consecutive days of a 28 days schedule. Further dosing regimens for HDM2 inhibitors are disclosed in WO 2015/198266.

Finding a safe but effective dose and dosage regimen for a specific HDM2 inhibitor in a specific therapeutic setting (single agent therapy or combination therapy, type of indication) remains a big challenge for the clinical use of those inhibitors.

### SUMMARY

The present invention provides COMPOUND A, or a pharmaceutically acceptable salt, solvate, complex or co-crystal thereof, as component in a combination with a PD-1 inhibitor, for use in the treatment of a cancer which is a TP53 wildtype cancer, particularly a TP53 wildtype solid tumor.

COMPOUND A is the compound with the following project code, chemical name and structure:

HDM201 (INN: siremadlin), i.e. (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, also referred to as (6S)-5-(5-Chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one,

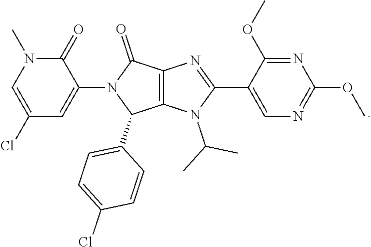 [SMILES: COc1ncc(-c2nc3c(n2C(C)C)[C@H](c2ccc(Cl)cc2)N(c2cc(Cl)cn(C)c2=O)C3=O)c(OC)n1]

Preferably, HDM201 is in the succinic acid co-crystal form. More preferably, HDM201 is in the 1:1 (molar ratio) succinic acid co-crystal form.

The present invention provides a pharmaceutical combination which comprises (a) at least one antibody molecule (e.g., humanized antibody molecules) that binds to Programmed Death 1 (PD-1), especially the exemplary antibody molecule as described below, and (b) a HDM2-p53 inhibitor which is Compound A, or pharmaceutically acceptable salt, solvate, complex or co-crystal thereof. The pharmaceutical combination may be used for the simultaneous, separate or sequential administration for the treatment of a proliferative disease, particularly a TP53 wildtype cancer, more particularly a TP53 wildtype solid tumor.

The present invention also relates to a pharmaceutical combination comprising (A) a HDM2-p53 inhibitor which is COMPOUND A (HDM201, siremadlin), or pharmaceutically acceptable salt, solvate, complex or co-crystal thereof; and (B) an isolated antibody molecule capable of binding to a human Programmed Death-1 (PD-1) comprising a heavy chain variable region (VH) comprising a HCDR1, a HCDR2 and a HCDR3 amino acid sequence of BAP049-Clone-B or BAP049-Clone-E as described in Table 1 and a light chain variable region (VL) comprising a LCDR1, a LCDR2 and a LCDR3 amino acid sequence of BAP049-Clone-B or BAP049-Clone-E as described in Table 1 below, preferably the anti-PD-1 antibody molecule is PDR001 (spartalizumab).

There is also provided a pharmaceutical composition comprising such a combination; a method of treating a subject having a proliferative disease comprising administration of said combination to a subject in need thereof; use of such combination for the treatment of proliferative disease; and a commercial package comprising such combination.

The PD-1 inhibitor is an anti-PD-1 antibody molecule as described in U.S. Ser. No. 14/604,415, entitled “Antibody Molecules to PD-1 and Uses Thereof,” and WO/2015/112900, both incorporated by reference in its entirety. In one embodiment, the anti-PD-1 antibody molecule comprises at least one antigen-binding region, e.g., a variable region or an antigen-binding fragment thereof, from an antibody described herein, including the three complementarity determining regions (CDRs) from the heavy and the three CDRs from the light chain, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

For example, the anti-PD-1 antibody molecule can include VH CDR1 according to Kabat et al. or VH hypervariable loop 1 according to Chothia et al., or a combination thereof, e.g., as shown in Table 1. In one embodiment, the combination of Kabat and Chothia CDR of VH CDR1 comprises the amino acid sequence GYTFTTYWMH (SEQ ID NO: 224), or an amino acid sequence substantially identical thereto (e.g., having at least one amino acid alteration, but not more than two, three or four alterations (e.g., substitutions, deletions, or insertions, e.g., conservative substitutions)). The anti-PD-1 antibody molecule can further include, e.g., VH CDRs 2-3 according to Kabat et al. and VL CDRs 1-3 according to Kabat et al., e.g., as shown in Table 1. Accordingly, in some embodiments, framework regions are defined based on a combination of CDRs defined according to Kabat et al. and hypervariable loops defined according to Chothia et al. For example, the anti-PD-1 antibody molecule can include VH FR1 defined based on VH hypervariable loop 1 according to Chothia et al. and VH FR2 defined based on VH CDRs 1-2 according to Kabat et al., e.g., as shown in Table 1. The anti-PD-1 antibody molecule can further include, e.g., VH FRs 3-4 defined based on VH CDRs 2-3 according to Kabat et al. and VL FRs 1-4 defined based on VL CDRs 1-3 according to Kabat et al.

A preferred antibody molecule (e.g., humanized antibody molecules) that binds to Programmed Death 1 (PD-1) in the combination of the present invention is the exemplary antibody molecule which is BAP049-Clone-E and the preferred amino acid sequences are described in Table 1 herein (VH: SEQ ID NO: 38; VL: SEQ ID NO: 70). The preferred antibody molecule is also referred herein as Antibody B or Spartalizumab (INN) or PDR001.

The present invention further provides a pharmaceutical combination comprising a HDM2-p53 inhibitor, which is COMPOUND A, or a pharmaceutically acceptable salt, solvate, complex or co-crystal thereof, and an anti-PD-1 antibody molecule, as described herein, for simultaneous, separate or sequential administration, for use in the treatment of a proliferative disease.

The present invention is particularly related to the combination of the invention for use in the treatment of a proliferative disease.

The present invention also provides the use of the combination of the invention for the treatment of a proliferative disease, particularly a cancer. In particular, the combination of the invention may be useful for the treatment of a cancer which is TP53 wildtype, in particular a TP53 solid tumor, and in particularly said TP53 solid tumor is selected from renal cell carcinoma (RCC) and colorectal cancer (CRC).

The present invention also provides the use of the combination of the invention for the preparation of a medicament for the treatment of a proliferative disease, particularly a cancer, particularly a cancer which is TP53 wildtype, in particular a TP53 solid tumor, and in particularly said TP53 solid tumor is selected from renal cell carcinoma (RCC) and colorectal cancer (CRC).

The present invention also provides a method of treating a proliferative disease comprising simultaneously, separately or sequentially administering to a subject in need thereof a combination of the invention in a quantity which is jointly therapeutically effective against said proliferative disease.

The present invention also provides a pharmaceutical composition or combined preparation comprising a quantity of the combination of the invention, which is jointly therapeutically effective against a proliferative disease, and optionally at least one pharmaceutically acceptable carrier.

The present invention also provides a combined preparation comprising (a) one or more dosage units of a HDM2 inhibitor, which is COMPOUND A, or a pharmaceutically acceptable salt thereof, and (b) an anti-PD-1 antibody molecule, for use in the treatment of a proliferative disease.

The present invention also provides a commercial package comprising as active ingredients a combination of the invention and instructions for simultaneous, separate or sequential administration of a combination of the invention to a patient in need thereof for use in the treatment of a proliferative disease, particularly a solid tumor that is TP53 wildtype.

The present invention also provides a commercial package comprising a HDM2 inhibitor, which is COMPOUND A, or a pharmaceutically acceptable salt, complex or co-crystal thereof, and an anti-PD-1 antibody molecule, and instructions for the simultaneous, separate or sequential use in the treatment of a proliferative disease.

In another aspect, the invention features diagnostic or therapeutic kits that include the antibody molecules and/or the low molecular weight active ingredients described herein and instructions for use.

The present invention also provides dose ranges and dosing regimens for the administration of the PD-1 inhibitor and HDM2 inhibitor.

In particular the present invention provides the combination of the PD-1 inhibitors as described herein and the HDM2 inhibitor HDM201 for use in the treatment of cancer, wherein the PD-1 inhibitor is dosed once every 4 weeks (q4w) and HDM201 is dosed on day 1, and on either one of days 6 to 14, preferably on either one of days 6 to 10, more preferably on day 8, of a 4 week treatment cycle (dId8q4w).

The daily dose of the PD-1 inhibitor is from 100 to 400 mg, preferably from 200 to 400 mg, more preferably from 300 to 400 mg, even more preferably the daily dose is 400 mg, and the daily dose of HDM201 is from 30 to 120 mg, preferably the daily dose is from 40 to 120 mg, more preferably the daily dose is from 60 to 120 mg, even more preferably the daily dose is from 60 mg to 90 mg, even more preferably the daily dose is from 60 to 80 mg. Herein, the daily dose of HDM201 refers to the free form, i.e. not including the mass any salt, solvate, complex or co-crystal former, e.g. not including the mass of the succinic acid in case of the HDM201 succinic acid co-crystal.

All publications, patent applications, patents, and other references mentioned herein are incorporated by reference in their entirety.

Other features, objects, and advantages of the invention will be apparent from the description and drawings, and from the claims.

### BRIEF DESCRIPTION OF THE DRAWINGS

FIG. 1 depicts the amino acid sequences of the light and heavy chain variable regions of murine anti-PD-1 mAb BAP049. The upper and lower sequences were from two independent analyses. The light and heavy chain CDR sequences based on Kabat numbering are underlined. The light heavy chain CDR sequences based on Chothia numbering are shown in bold italics. The unpaired Cys residue at position 102 of the light chain sequence is boxed. Sequences are disclosed as SEQ ID NOs: 8, 228, 16 and 229, respectively, in order of appearance.

FIG. 2A depicts the amino acid sequences of the light and heavy chain variable regions of murine anti-PD-1 mAb BAP049 aligned with the germline sequences. The upper and lower sequences are the germline (GL) and BAP049 (Mu mAb) sequences, respectively. The light and heavy chain CDR sequences based on Kabat numbering are underlined. The light heavy chain CDR sequences based on Chothia numbering are shown in bold italics. “-” means identical amino acid residue. Sequences disclosed as SEQ ID NOs: 230, 8, 231 and 16, respectively, in order of appearance.

FIG. 2B depicts the sequence of murine κ J2 gene and the corresponding mutation in murine anti-PD-1 mAb BAP049. “-” means identical nucleotide residue. Sequences disclosed as SEQ ID NOs: 233, 232, 234 and 235, respectively, in order of appearance.

FIGS. 3A-3B depict the competition binding between fluorescently labeled murine anti-PD-1 mAb BAP049 (Mu mAb) and three chimeric versions of BAP049 (Chi mAb). Experiment was performed twice, and the results are shown in FIGS. 3A and 3B, respectively. The three chimeric BAP049 antibodies (Chi mAb (Cys), Chi mAb (Tyr) and Chi mAb (Ser)) have Cys, Tyr and Ser residue at position 102 of the light chain variable region, respectively. Chi mAb (Cys), Chi mAb (Tyr) and Chi mAb (Ser) are also known as BAP049-chi, BAP049-chi-Y, and BAP049-chi-S, respectively.

FIG. 4 is a bar graph showing the results of FACS binding analysis for the sixteen humanized BAP049 clones (BAP049-hum01 to BAP049-hum16). The antibody concentrations are 200, 100, 50, 25 and 12.5 ng/ml from the leftmost bar to the rightmost bar for each tested mAb.

FIG. 5 depicts the structural analysis of the humanized BAP049 clones (a, b, c, d and e represent various types of framework region sequences). The concentrations of the mAbs in the samples are also shown.

FIG. 6A-6B depicts the binding affinity and specificity of humanized BAP049 mAbs measured in a competition binding assay using a constant concentration of Alexa 488-labeled murine mAb BAP049, serial dilutions of the test antibodies, and PD-1-expressing 300.19 cells. Experiment was performed twice, and the results are shown in FIGS. 6A and 6B, respectively.

FIG. 7 depicts the ranking of humanized BAP049 clones based on FACS data, competition binding and structural analysis. The concentrations of the mAbs in the samples are also shown.

FIGS. 8A-8B depict blocking of ligand binding to PD-1 by selected humanized BAP049 clones. Blocking of PD-LI-Ig and PD-L2-Ig binding to PD-1 is shown in FIG. 8A. Blocking of PD-L2-Ig binding to PD-1 is shown in FIG. 8B. BAP049-hum01, BAP049-hum05, BAP049-hum08, BAP049-hum09, BAP049-hum10, and BAP049-hum11 were evaluated. Murine mAb BAP049 and chimeric mAb having Tyr at position 102 of the light chain variable region were also included in the analyses.

FIGS. 9A-9B depict the alignment of heavy chain variable domain sequences for the sixteen humanized BAP049 clones and BAP049 chimera (BAP049-chi). In FIG. 9A, all of the sequences are shown (SEQ ID NOs: 22, 38, 38, 38, 38, 38, 38, 38, 38, 38, 50, 50, 50, 50, 82, 82 and 86, respectively, in order of appearance). In FIG. 9B, only amino acid sequences that are different from mouse sequence are shown (SEQ ID NOs: 22, 38, 38, 38, 38, 38, 38, 38, 38, 38, 50, 50, 50, 50, 82, 82 and 86, respectively, in order of appearance).

FIGS. 10A-10B depict the alignment of light chain variable domain sequences for the sixteen humanized BAP049 clones and BAP049 chimera (BAP049-chi). In FIG. 10A, all of the sequences are shown (SEQ ID NOs: 24, 66, 66, 66, 66, 70, 70, 70, 58, 62, 78, 74, 46, 46, 42, 54 and 54, respectively, in order of appearance). In FIG. 10B, only amino acid sequences that are different from mouse sequence are shown (SEQ ID NOs: 24, 66, 66, 66, 66, 70, 70, 70, 58, 62, 78, 74, 46, 46, 42, 54 and 54, respectively, in order of appearance).

FIG. 11 is a schematic diagram that outlines the antigen processing and presentation, effector cell responses and immunosuppression pathways targeted by the combination therapies disclosed herein.

FIG. 12 depicts the predicted Ctrough (Cmin) concentrations across the different weights for patients while receiving the same dose of an exemplary anti-PD-1 antibody molecule.

FIG. 13 depicts observed versus model predicted (population or individual based) Cmin concentrations.

FIG. 14 depicts the accumulation, time course and within subject variability of the model used to analyze pharmacokinetics.

FIG. 15 shows the average concentration per cycle estimated for patients treated at 120 mg on regimen 1B. Cohort 1: 120 mg. cohort 2: 120 mg, new variant. Dashed line: Tumor stasis (SJSA-1 cell line), Dotted line: Tumor stasis (liposarcoma cell line). Each individual patient is represented by a circle.

FIG. 16 shows the geometric mean concentration-time profile (Regimen 1A, Cycle 1 Day 1) (PAS).

FIG. 17 shows the Individual human average NVP-HDM201 concentration during first cycle (DDS). Individual C (average)=individual AUC mode at the end of Cycle 1 divided by duration of Cycle 1 in hours. Average dose level=total cumulative dose at the end of Cycle 1 divided by the duration of Cycle 1 in days.

FIG. 18 shows the platelet kinetic profiles modeled based on the following doses as tested in each regimen (in order from top to bottom): Reg2C (D1-7 Q4 wk): 25 mg (6.25 mg/d); Reg2A (D1-14 Q4 wk): 20 mg (10 mg/d); Reg1B (Days 1, 8 Q4 wk): 150 mg (10.7 mg/d); Reg1A (D1 Q3 wk): 350 mg (16.7 mg/d).

FIG. 19 shows the individual average concentration during first treatment cycle versus dose per regimen for patients with hematological tumors.

Line at 120 ng/mL=95% tumor regression from human SJSA-1 xenograft rat. Line at 41 ng/mL=Average concentration for tumor stasis derived from TGI PK/PD modelling in human SJSA-1 (osteosarcoma) xenograft rat. Line at 19 ng/mL=Average concentration for tumor stasis derived from TGI PK/PD modelling in human HSAX2655 (liposarcoma) PDX rat.

Calculation of average dose level (mg/day):

| | Daily | No. of | Total dose | Cycle | Average |
|---|---|---|---|---|---|
| | dose | administration | per cycle | duration | dose |
| Regimen | (mg) | days | (mg) | (days) | (mg/day) |
| 1A | 250 | 1 | 250 | 21 | 11.9 |
| | 350 | 1 | 350 | 21 | 16.7 |
| | 400 | 1 | 400 | 21 | 19 |
| 1B | 150 | 2 | 300 | 28 | 10.7 |
| 2A | 20 | 14 | 280 | 28 | 10 |
| | 30 | 14 | 420 | 28 | 15 |
| 2C | 45 | 7 | 315 | 28 | 11.3 |

FIG. 20 shows the best percentage change from baseline in sum of diameter and best overall response for sarcoma (liposarcoma and other sarcomas) patients treated with HDM201 according to regimen 1B (September 2017). PD: progressing disease, SD: stable disease, PR: partial response.

FIG. 21: HDM201 Modulated Immune Cell Infiltrates in Colon26 Tumors in Balb/c Mice (7628 Colon 26-XPD)

HDM201 modulated profiles of immune cells in Colon 26 tumors. Increases in % CD11c⁺/CD45⁺ myeloid cells (A), % CD8⁺/CD45⁺ T cells (B), PDL1 MFI in CD45⁻ cells (C), and % PD1⁺/CD45⁺ lymphocytes (d). Colon 26 cells were implanted into the right flank of Balb/c mice. When tumors reached ˜60 mm³, mice were randomized and treated with HDM201 at 40 mg/kg every 3h for 3 times on days 0 and 7. Mice were euthanized, and tumors were collected and processed for FACS analysis on Days 5 and 12 post first dose.

FIG. 22: HDM201 Enhanced DC function, T Cell Priming and CD8/T_{reg} Ratio in Colon 26 Tumors and Draining Lymph Nodes (8063 Colon 26-XPD)

HDM201 modulated profiles of immune cells in Colon 26 tumors. Increases in % CD103⁺CD11c⁺ DCs (A), % Tbet*EOMES-CD8+/CD45⁺ T cells (B), and CD8/Treg ratio (C). Colon 26 Cells were implanted into right flank of Balb/c mice. When tumors reached ˜100 mm³, mice were randomized and treated with HDM201 at 40 mg/kg every 3h for 3 times on days 0 and 7. Mice were euthanized; tumors and draining lymph nodes were collected and processed for FACS analysis on Days 5 and 12 post first dose.

FIG. 23: Percent Body Weight Change (8020 Colon 26-XEF)

Percent body weight change. Balb/c mice were implanted with 2×10 Colon 26 cells subcutaneously. Mice were treated with HDM201 at 40 mg/kg×3 every 3h po on Days 12, 19 and 26 post cell implant, and the aPD-1 antibody at 5 mg/kg ip on days 12, 15, 19, and 22. Body weight was recorded twice a week, and percent body change was calculated based on the formula described in the corresponding section of example 3.

FIG. 24: Time to Endpoint (8020 Colon 26-XEF)

Time to endpoint. Balb/c mice were implanted with 2×10⁵ Colon 26 cells subcutaneously. Mice were treated with HDM201 at 40 mg/kg×3 for every 3h po on Days 12, 19 and 26 post cell implant, and the aPD-1 antibody at 5 mg/kg ip on days 12, 15, 19, and 22. End point was defined as tumor volume equal or greater than 1000 mm³. Log Rank, p<0.05.

FIG. 25: Individual Tumor Growth Curves (8020 Colon 26-XEF) Individual tumor growth curves. Balb/c mice were implanted with 2×10⁵ Colon 26 cells subcutaneously. Mice were treated with HDM201 at 40 mg/kg×3 for every 3h po on Days 12, 19 and 26 post cell implant, and the aPD-1 antibody at 5 mg/kg ip on days 12, 15, 19, and 22. End point was defined as tumor volume equal to or greater than 1000 mm³. The horizontal dashed line indicates the tumor endpoint tumor size (1000 mm³).

FIG. 26: Mice Developed Long Term Specific Memory to Colon 26 Cells, but not 4T1 Cells (8020 Colon 26-XEF).

Long term specific memory was developed in CR mice previously treated with the combination of HDM201 with aPD1 antibody. A) All mice that had achieved CR after HDM201+aPD1 antibody treatment rejected the second injection of Colon 26 cells. Naive mice (n=5) and CR mice (HDM201+aPD1 Ab, n=5) were implanted with 2×10⁵ Colon 26 cells on the left side of the flank. Tumor volume was measured weekly. No tumor was observed until Day 34 in mice with CR. B) Six weeks later, 4T1 cells were implanted into the mammary fat pad of naive mice (n=5) and CR mice (HDM201+aPD1 Ab, n=5). Tumor volumes were measured, all mice developed 4T1 tumors, and were euthanized on Day 14 post 4T1 cell implant.

FIG. 27: Demonstration of the memory effect by re-challenging animals with colon 26 and 4T1 cells.

FIG. 28: Demonstration of the anti-tumor memory T cell responses: frequency of AH1-specific CD8⁺ T cells in spleens of mice treated with HDM201 or combination of HDM201 with anti-PD1 antibody induced responders as detected by H2Ld-AH1 dextramers.

FIG. 29: Demonstration of the anti-tumor memory T cell responses: Frequency of CD44+AH1+ within CD8⁺ T cells.

FIG. 30: In vitro characterization of p53 knock out colon 26 clones

FIG. 31: Study periods of the clinical study CPDR001X2102

### BRIEF DESCRIPTION OF THE TABLES

Table 1 is a summary of the amino acid and nucleotide sequences for the murine, chimeric and humanized anti-PD-1 antibody molecules. The antibody molecules include murine mAb BAP049, chimeric mAbs BAP049-chi and BAP049-chi-Y, and humanized mAbs BAP049-hum01 to BAP049-hum16 and BAP049-Clone-A to BAP049-Clone-E. The amino acid and nucleotide sequences of the heavy and light chain CDRs, the amino acid and nucleotide sequences of the heavy and light chain variable regions, and the amino acid and nucleotide sequences of the heavy and light chains are shown in this Table.

Table 2 depicts the amino acid and nucleotide sequences of the heavy and light chain framework regions for humanized mAbs BAP049-hum01 to BAP049-hum16 and BAP049-Clone-A to BAP049-Clone-E.

Table 3 depicts the constant region amino acid sequences of human IgG heavy chains and human kappa light chain.

Table 4 shows the amino acid sequences of the heavy and light chain leader sequences for humanized mAbs BAP049-Clone-A to BAP049-Clone-E.

Table 5 depicts exemplary PK parameters based on flat dosing schedules.

### DETAILED DESCRIPTION

HDM2 Inhibitor

The term “HDM2 inhibitor”, also referred to as “HDM2i”, “Hdm2i”, “MDM2 inhibitor”, “MDM2i”, “Mdm2i”, denotes herein any compound inhibiting the HDM-2/p53 or HDM-4/p53 interaction with an IC50 of less than 10 μM, preferably less than 1 μM, preferably in the range of nM, measured by a Time Resolved Fluorescence Energy Transfer (TR-FRET) Assay. The inhibition of p53-Hdm2 and p53-Hdm4 interactions is measured by time resolved fluorescence energy transfer (TR-FRET). Fluorescence energy transfer (or Foerster resonance energy transfer) describes an energy transfer between donor and acceptor 5 fluorescent molecules. For this assay, MDM2 protein (amino acids 2-188) and MDM4 protein (amino acids 2-185), tagged with a C-terminal Biotin moiety, are used in combination with a Europium labeled streptavidin (Perkin Elmer, Inc., Waltham, Mass., USA) serving as the donor fluorophore. The p53 derived, Cy5 labeled peptide Cy5-TFSDLWKLL (p53 aa18-26) is the energy acceptor. Upon excitation of the donor 10 molecule at 340 nm, binding interaction between MDM2 or MDM4 and the p53 peptide induces energy transfer and enhanced response at the acceptor emission wavelength at 665 nm. Disruption of the formation of the p53-MDM2 or p53-MDM4 complex due to an inhibitor molecule binding to the p53 binding site of MDM2 or MDM4 results in increased donor emission at 615 nm. The ratiometric FRET assay readout is calculated from the 15 raw data of the two distinct fluorescence signals measured in time resolved mode (countrate 665 nm/countrate 615 nm×1000). The assay can be performed according to the following procedure: The test is performed in white 1536w microtiterplates (Greiner Bio-One GmbH, Frickenhausen, Germany) in a total volume of 3.1 μl by combining 100 nl of compounds diluted in 90% DMSO/10% H₂O (3.2% final DMSO concentration) with 2 μl Europium 20 labeled streptavidin (final concentration 2.5 nM) in reaction buffer (PBS, 125 mM NaCl, 0.001% Novexin (consists of carbohydrate polymers (Novexin polymers), designed to increase the solubility and stability of proteins; Novexin Ltd., ambridgeshire, United Kingdom), Gelatin 0.01%, 0.2% Pluronic (block copolymer from ethylenoxide and propyleneoxide, BASF, Ludwigshafen, Germany), 1 mM DTT), followed by the addition of 0.5 μl MDM2-Bio or MDM4-Bio diluted in assay buffer (final concentration 10 nM). Allow the solution to pre-incubate for 15 minutes at room temperature, followed by addition of 0.5 μl Cy5-p53 peptide in assay buffer (final concentration 20 nM). Incubate at room temperature for 10 minutes prior to reading the plate. For measurement of samples, an Analyst GT multimode microplate reader (Molecular Devices) with the following settings 30 is used: Dichroic mirror 380 nm, Excitation 330 nm, Emission Donor 615 nm and Emission Acceptor 665 nm. IC50 values are calculated by curve fitting using XLfit. If not specified, reagents are purchased from Sigma Chemical Co, St. Louis, Mo., USA.

The preferred HDM2 inhibitor according to the present invention is HDM201, i.e. (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, also referred to as (6S)-5-(5-Chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one,

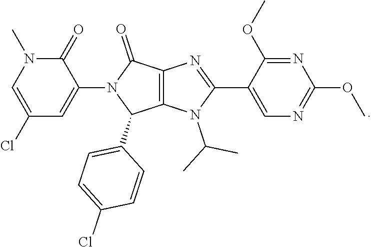 [SMILES: COc1ncc(-c2nc3c(n2C(C)C)[C@H](c2ccc(Cl)cc2)N(c2cc(Cl)cn(C)c2=O)C3=O)c(OC)n1]

HDM201 may be present as free molecule, as solvate (incl. hydrate) or as acid variant. The solvate may be an ethanol solvate (ethanolate). The acid variant may be a salt formed of HDM201 with the acid, or a HDM201 acid complex, or as HDM201 acid co-crystal, preferably HDM201 is present as co-crystal. Preferable the acid is succinic acid. Most preferably, the HDM201 is present as succinic acid co-crystal.

HDM201 and its hydrates, solvates and acid variants and manufacturing processes thereof are described in WO2013/111105 (e.g. example 102, forms A, B, and C).

Antibody Molecules to PD-1

In one embodiment, the PD-1 inhibitor is an anti-PD-1 antibody molecule as described in U.S. Ser. No. 14/604,415, entitled “Antibody Molecules to PD-1 and Uses Thereof,” and WO/2015/112900, both incorporated by reference in its entirety. In one embodiment, the anti-PD-1 antibody molecule comprises at least one antigen-binding region, e.g., a variable region or an antigen-binding fragment thereof, from an antibody described herein, including the three complementarity determining regions (CDRs) from the heavy and the three CDRs from the light chain, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

For example, the anti-PD-1 antibody molecule can include VH CDR1 according to Kabat et al. or VH hypervariable loop 1 according to Chothia et al., or a combination thereof, e.g., as shown in Table 1. In one embodiment, the combination of Kabat and Chothia CDR of VH CDR1 comprises the amino acid sequence GYTFTTYWMH (SEQ ID NO: 224), or an amino acid sequence substantially identical thereto (e.g., having at least one amino acid alteration, but not more than two, three or four alterations (e.g., substitutions, deletions, or insertions, e.g., conservative substitutions)). The anti-PD-1 antibody molecule can further include, e.g., VH CDRs 2-3 according to Kabat et al. and VL CDRs 1-3 according to Kabat et al., e.g., as shown in Table 1. Accordingly, in some embodiments, framework regions are defined based on a combination of CDRs defined according to Kabat et al. and hypervariable loops defined according to Chothia et al. For example, the anti-PD-1 antibody molecule can include VH FR1 defined based on VH hypervariable loop 1 according to Chothia et al. and VH FR2 defined based on VH CDRs 1-2 according to Kabat et al., e.g., as shown in Table 1. The anti-PD-1 antibody molecule can further include, e.g., VH FRs 3-4 defined based on VH CDRs 2-3 according to Kabat et al. and VL FRs 1-4 defined based on VL CDRs 1-3 according to Kabat et al.

A preferred antibody molecule (e.g., humanized antibody molecule) that binds to Programmed Death 1 (PD-1) in the combination of the present invention is the exemplary antibody molecule which is BAP049-Clone-E and the preferred amino acid sequences are described in Table 1 herein (VH: SEQ ID NO: 38; VL: SEQ ID NO: 70). This particularly preferred antibody molecule is herein also referred to as PDR001 or spartalizumab (INN).

The present invention further relates to a pharmaceutical combination comprising (a) at least one antibody molecule (e.g., humanized antibody molecules) that binds to Programmed Death 1 (PD-1), especially the exemplary antibody molecule as described herein, and (b) a HDM2 inhibitor, such as Compound A, or pharmaceutically acceptable salt, solvate, complex, or co-crystal thereof, for simultaneous, separate or sequential administration for the treatment of a proliferative disease, particularly a TP53 wildtype solid tumor.

In one embodiment, the invention features a method of treating (e.g., inhibiting, reducing, or ameliorating) a disorder, e.g., a hyperproliferative condition or disorder (e.g., a cancer) in a subject. The method includes administering, in combination with a HDM2 inhibitor, to the subject an anti-PD-1 antibody molecule, e.g., the preferred anti-PD-1 antibody molecule described herein, at a dose of about 300 mg to 400 mg once every three weeks or once every four weeks. In certain embodiments, the e.g., the preferred anti-PD-1 antibody molecule is administered at a dose of about 300 mg once every three weeks. In other embodiments, the e.g., the preferred anti-PD-1 antibody molecule is administered at a dose of about 400 mg once every four weeks. In some embodiments, the proliferative disorder is a cancer. In some embodiments, the proliferative disorder is a TP53 wildtype tumor and in particular, TP53 wildtype solid tumor.

To be considered TP53 wildtype a tumor must at a minimum have no mutations detected in exons 5, 6, 7 and 8 in a tumor sample collected no longer than 36 months before the first dose of study drug. Tumors previously documented as having genomic amplification of HDM2 (defined as >4 copy number, irrespective of the date) do not require TP53 WT status confirmation.

In some embodiments, the proliferative disorder is a TP53 wildtype RCC.

In some embodiments, the proliferative disorder is a TP53 wildtype CRC, in particular a microsatellite stable (MSS) CRC, also referred to as MSS CRC.

In some embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. The dosing schedule (e.g., flat dosing schedule) can vary from e.g., once a week to once every 2, 3, 4, 5, or 6 weeks. In one embodiment, the anti-PD-1 antibody molecule, e.g., the exemplary antibody molecule, is administered at a dose from about 300 mg to 400 mg once every three weeks or once every four weeks. In one embodiment, the anti-PD-1 antibody molecule is administered at a dose of about 300 mg once every three weeks. In one embodiment, the anti-PD-1 antibody molecule is administered at a dose of about 400 mg once every four weeks. In one embodiment, the anti-PD-1 antibody molecule, e.g., the exemplary antibody molecule, is administered at a dose from about 300 mg once every four weeks. In one embodiment, the the anti-PD-1 antibody molecule, e.g., the exemplary antibody molecule, is administered at a dose from about 400 mg once every three weeks.

In another aspect, the invention features a method of reducing an activity (e.g., growth, survival, or viability, or all), of a hyperproliferative (e.g., a cancer) cell. The method includes contacting the cell with an anti-PD-1 antibody molecule, e.g., an anti-PD-1 antibody molecule described herein. The method can be performed in a subject, e.g., as part of a therapeutic protocol in combination with a c-Raf receptor tyrosine kinase inhibitor, e.g., at a dose of about 300 mg to 400 mg of an anti-PD-1 antibody molecule once every three weeks or once every four weeks. In certain embodiments, the dose is about 300 mg of an anti-PD-1 antibody molecule once every three weeks. In other embodiments, the dose is about 400 mg of an anti-PD-1 antibody molecule once every four weeks.

In another aspect, the invention features a composition (e.g., one or more compositions or dosage forms), that includes an anti-PD-1 antibody molecule (e.g., an anti-PD-1 antibody molecule as described herein). Formulations, e.g., dosage formulations, and kits, e.g., therapeutic kits, that include an anti-PD-1 antibody molecule (e.g., an anti-PD-1 antibody molecule as described herein), are also described herein. In certain embodiments, the composition or formulation comprises 300 mg or 400 mg of an anti-PD-1 antibody molecule (e.g., an anti-PD-1 antibody molecule as described herein). In some embodiments, the composition or formulation is administered or used once every three weeks or once every four weeks. Such composition is used in combination with a HDM2 inhibitor or pharmaceutically acceptable salt, solvate, complex or co-crystal thereof, for simultaneous, separate or sequential administration, often for treatment of RCC or CRC, and particularly for treating a patient having RCC or MSS CRC.

In another aspect, the invention provides an anti-PD-1 antibody for use in treating RCC or CRC, wherein the anti-PD-1 antibody is administered, or prepared for administration, separately, simultaneously, or sequentially with a HDM2 inhibitor. It also provides a HDM2 inhibitor for use in treating RCC or CRC, wherein the HDM2 inhibitor is administered, or prepared for administration, separately, simultaneously, or sequentially with an anti-PD-1 antibody.

Typically, the anti-PD-1 antibody is administered intravenously, and is thus administered separately or sequentially with the HDM2 inhibitor, which is preferably administered orally. Suitable methods, routes, dosages and frequency of administration of the HDM2 inhibitor and the anti-PD-1 antibody are described herein.

The combinations disclosed herein can be administered together in a single composition or administered separately in two or more different compositions, e.g., compositions or dosage forms as described herein. The administration of the therapeutic agents can be in any order. The first agent and the additional agents (e.g., second, third agents) can be administered via the same administration route or via different administration routes.

The pharmaceutical combinations described herein, in particular the pharmaceutical combination of the invention, may be a free combination product, i.e. a combination of two or more active ingredients, e.g. COMPOUND A and the exemplary antibody molecule described herein (Antibody B), which is administered simultaneously, separately or sequentially as two or more distinct dosage forms.

A free combination product can be: (a) two or more separate drug products packaged together in a single package or kit, or (b) a drug product packaged separately that according to its labelling is for use only with other individually specified drugs where each drug is required to achieve the intended use, indication, or effect.

The present invention also provides a combined preparation comprising (a) one or more dosage units of the HDM2 inhibitor Compound A, or a pharmaceutically acceptable salt thereof, and (b) one or more dosage units of an anti-PD-1 antibody as described herein, and at least one pharmaceutically acceptable carrier.

In a further embodiment, the present invention is particularly related to a method of treating a proliferative disease, particularly a cancer. In one embodiment, the present invention relates to the use of the combination of the invention for the preparation of a medicament for the treatment of a proliferative disease, particularly a cancer. In one embodiment, the combination of the invention is for use in the preparation of a medicament for the treatment of a proliferative disease, particularly a cancer.

The present invention also provides a pharmaceutical combination described herein, e.g. the pharmaceutical combination comprising (a) COMPOUND A, or a pharmaceutically acceptable salt, solvate, complex or co-crystal thereof, and (b) an isolated antibody molecule capable of binding to a human Programmed Death-1 (PD-1) comprising a heavy chain variable region (VH) comprising a HCDR1, a HCDR2 and a HCDR3 amino acid sequence of BAP049-Clone-B or BAP049-Clone-E as described in Table 1 and a light chain variable region (VL) comprising a LCDR1, a LCDR2 and a LCDR3 amino acid sequence of BAP049-Clone-B or BAP049-Clone-E as described in Table 1 below—for use in the treatment of a TP53 wildtype solid tumor.

Uses of the Combination Therapies

The combinations disclosed herein can result in one or more of: an increase in antigen presentation, an increase in effector cell function (e.g., one or more of T cell proliferation, IFN-γ secretion or cytolytic function), inhibition of regulatory T cell function, an effect on the activity of multiple cell types, such as regulatory T cell, effector T cells and NK cells), an increase in tumor infiltrating lymphocytes, an increase in T-cell receptor mediated proliferation, and a decrease in immune evasion by cancerous cells. In one embodiment, the use of a PD-1 inhibitor in the combination inhibits, reduces or neutralizes one or more activities of PD-1, resulting in blockade or reduction of an immune checkpoint. Thus, such combinations can be used to treat or prevent disorders where enhancing an immune response in a subject is desired.

Accordingly, in another aspect, a method of modulating an immune response in a subject is provided. The method comprises administering to the subject a combination disclosed herein (e.g., a combination comprising a therapeutically effective amount of an anti-PD-1 antibody molecule and a therapeutically effective amount of COMPOUND A, or a pharmaceutically acceptable salt, solvate, complex or co-crystal thereof), such that the immune response in the subject is modulated. In one embodiment, the antibody molecule enhances, stimulates or increases the immune response in the subject. The subject can be a mammal, e.g., a primate, preferably a higher primate, e.g., a human (e.g., a patient having, or at risk of having, a disorder described herein). In one embodiment, the subject is in need of enhancing an immune response. In one embodiment, the subject has, or is at risk of, having a disorder described herein, e.g., a cancer or an infectious disorder as described herein. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

In one aspect, a method of treating (e.g., one or more of reducing, inhibiting, or delaying progression) proliferative disease which is a solid tumor that it TP53 wildtype, in particular RCC or CRC. In another aspect, a method of treating (e.g., one or more of reducing, inhibiting, or delaying progression) proliferative disease which is a solid tumor that is TP53 wildtype, in particular, RCC or CRC in a subject is provided. The method comprises administering to the subject a combination disclosed herein (e.g., a combination comprising a therapeutically effective amount of an anti-PD-1 antibody molecule and a therapeutically effective amount of Compound A, or a pharmaceutically acceptable salt, solvate, complex or co-crystal thereof).

The combinations as described herein can be administered to the subject systemically (e.g., orally, parenterally, subcutaneously, intravenously, rectally, intramuscularly, intraperitoneally, intranasally, transdermally, or by inhalation or intracavitary installation), topically, or by application to mucous membranes, such as the nose, throat and bronchial tubes.

Dosages and Therapeutic Regimens

Dosages and therapeutic regimens of the therapeutic agents disclosed herein can be determined by a skilled artisan. In certain embodiments, the anti-PD-1 antibody molecule is administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks. In one embodiment, the anti-PD-1 antibody molecule is administered at a dose from about 10 to 20 mg/kg every other week.

In some embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. The dosing schedule (e.g., flat dosing schedule) can vary from e.g., once a week to once every 2, 3, 4, 5, or 6 weeks. In one embodiment, the anti-PD-1 antibody molecule is administered at a dose from about 300 mg to 400 mg once every three weeks or once every four weeks. In one embodiment, the anti-PD-1 antibody molecule is administered at a dose from about 300 mg once every three weeks. In one embodiment, the anti-PD-1 antibody molecule is administered at a dose from about 400 mg once every four weeks. In one embodiment, the anti-PD-1 antibody molecule is administered at a dose from about 300 mg once every four weeks. In one embodiment, the anti-PD-1 antibody molecule is administered at a dose from about 400 mg once every three weeks.

The total daily dose of COMPOUND A may be administered in a single dose (i.e. once daily) or twice daily. For example, COMPOUND A may be administered at a dose of 1200 mg once daily, or 400 mg twice daily.

The HDM2 inhibitor which is COMPOUND A may be administered on day 1 and day 8 of a 4 week treatment cycle at a daily dose of about 30, 40, 50, 60, 70, 80, 90, 100, 110, 120 mg and the preferred anti-PD-1 antibody molecule is administered at a dose of about 400 mg once every three weeks.

The HDM2 inhibitor which is COMPOUND A may be administered on day 1 and day 8 of a 4 week treatment cycle at a daily dose of about 30, 40, 50, 60, 70, 80, 90, 100, 110, 120 mg and the anti-PD-1 antibody molecule is administered at a dose of about 400 mg once every four weeks.

COMPOUND A may in particular be administered on day 1 and day 8 of a 4 week treatment cycle at a daily dose of about 40, 60, 80, 100, 120 mg at once daily (QD).

In a preferred embodiment, the exemplary anti-PD-1 molecule may be administered at a dose of 400 mg once every four weeks and COMPOUND A may be administered on day 1 and day 8 of a 4 week treatment cycle at a daily dose of 60, 80, 100, or 120 mg.

Further Combination Therapies

The methods and combinations described herein can be used in combination with other agents or therapeutic modalities. In one embodiment, the methods described herein include administering to the subject a combination comprising an anti-PD-1 antibody molecule as described herein, in combination with an agent or therapeutic procedure or modality, in an amount effective to treat or prevent a disorder. The anti-PD-1 antibody molecule and the agent or therapeutic procedure or modality can be administered simultaneously or sequentially in any order. Any combination and sequence of the anti-PD-1 antibody molecules and other therapeutic agents, procedures or modalities (e.g., as described herein) can be used. The antibody molecule and/or other therapeutic agents, procedures or modalities can be administered during periods of active disorder, or during a period of remission or less active disease. The antibody molecule can be administered before the other treatment, concurrently with the treatment, post-treatment, or during remission of the disorder.

In certain embodiments, the methods and compositions described herein are administered in combination with one or more of other antibody molecules, chemotherapy, other anti-cancer therapy (e.g., targeted anti-cancer therapies, gene therapy, viral therapy, RNA therapy bone marrow transplantation, nanotherapy, or oncolytic drugs), cytotoxic agents, immune-based therapies (e.g., cytokines or cell-based immune therapies), surgical procedures (e.g., lumpectomy or mastectomy) or radiation procedures, or a combination of any of the foregoing. The additional therapy may be in the form of adjuvant or neoadjuvant therapy. In some embodiments, the additional therapy is an enzymatic inhibitor (e.g., a small molecule enzymatic inhibitor) or a metastatic inhibitor. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteosome inhibitors, and radiation (e.g., local or whole body irradiation (e.g., gamma irradiation). In other embodiments, the additional therapy is surgery or radiation, or a combination thereof. In other embodiments, the additional therapy is a therapy targeting one or more of PI3K/AKT/mTOR pathway, an HSP90 inhibitor, or a tubulin inhibitor.

Alternatively, or in combination with the aforesaid combinations, the methods and compositions described herein can be administered in combination with one or more of: an immunomodulator (e.g., an activator of a costimulatory molecule or an inhibitor of an inhibitory molecule, e.g., an immune checkpoint molecule); a vaccine, e.g., a therapeutic cancer vaccine; or other forms of cellular immunotherapy.

In one embodiment, the combination disclosed herein, e.g., a combination comprising an anti-PD-1 antibody molecule, is used in combination with chemotherapy to treat a lung cancer, e.g., non-small cell lung cancer. In one embodiment, the anti-PD-1 antibody molecule is used with standard lung, e.g., NSCLC, chemotherapy, e.g., platinum doublet therapy, to treat lung cancer. The cancer may be at an early, intermediate or late stage.

In one embodiment, the combination disclosed herein, e.g., a combination comprising an anti-PD-1 antibody molecule, is used in combination with chemotherapy to treat skin cancer, e.g., melanoma. In one embodiment, the anti-PD-1 antibody molecule is used with standard skin, e.g., melanoma, chemotherapy, e.g., platinum doublet therapy, to treat skin cancer. The cancer may be at an early, intermediate or late stage.

Any combination and sequence of the anti-PD-1 antibody molecules and other therapeutic agents, procedures or modalities (e.g., as described herein) can be used. The antibody molecule and/or other therapeutic agents, procedures or modalities can be administered during periods of active disorder, or during a period of remission or less active disease. The antibody molecule can be administered before the other treatment, concurrently with the treatment, post-treatment, or during remission of the disorder.

Disclosed herein, at least in part, are antibody molecules (e.g., humanized antibody molecules) that bind to Programmed Death 1 (PD-1) with high affinity and specificity. Nucleic acid molecules encoding the antibody molecules, expression vectors, host cells and methods for making the antibody molecules are also provided. Pharmaceutical compositions and dose formulations comprising the antibody molecules are also provided. The anti-PD-1 antibody molecules disclosed herein can be used (alone or in combination with other agents or therapeutic modalities) to treat, prevent and/or diagnose disorders, such as cancerous disorders (e.g., solid and soft-tissue tumors). Thus, compositions and methods for detecting PD-1, as well as methods for treating various disorders including cancer using the anti-PD-1 antibody molecules are disclosed herein. In certain embodiments, the anti-PD-1 antibody molecule is administered or used at a flat or fixed dose.

### Definitions

Additional terms are defined below and throughout the application.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

By “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In embodiments, the additional therapeutic agent is administered at a therapeutic or lower-than therapeutic dose. In certain embodiments, the concentration of the second therapeutic agent that is required to achieve inhibition, e.g., growth inhibition is lower when the second therapeutic agent is administered in combination with the first therapeutic agent, e.g., the anti-PD-1 antibody molecule, than when the second therapeutic agent is administered individually. In certain embodiments, the concentration of the first therapeutic agent that is required to achieve inhibition, e.g., growth inhibition is lower when the first therapeutic agent is administered in combination with the second therapeutic agent than when the first therapeutic agent is administered individually. In certain embodiments, in a combination therapy, the concentration of the second therapeutic agent that is required to achieve inhibition, e.g., growth inhibition is lower than the therapeutic dose of the second therapeutic agent as a monotherapy, e.g., 10-20%, 20-30%, 30-40%, 40-50%, 50-60%, 60-70%, 70-80%, or 80-90% lower. In certain embodiments, in a combination therapy, the concentration of the first therapeutic agent that is required to achieve inhibition, e.g. growth inhibition, is lower than the therapeutic dose of the first therapeutic agent as a monotherapy, e.g., 10-20%, 20-30%, 30-40%, 40-50%, 50-60%, 60-70%, 70-80%, or 80-90% lower.

The term “inhibition,” “inhibitor,” or “antagonist” includes a reduction in a certain parameter, e.g., an activity, of a given molecule, e.g., an immune checkpoint inhibitor. For example, inhibition of an activity, e.g., a PD-1 or PD-L1 activity, of at least 5%, 10%, 20%, 30%, 40% or more is included by this term. Thus, inhibition need not be 100%.

The term “activation,” “activator,” or “agonist” includes an increase in a certain parameter, e.g., an activity, of a given molecule, e.g., a costimulatory molecule. For example, increase of an activity, e.g., a costimulatory activity, of at least 5%, 10%, 25%, 50%, 75% or more is included by this term.

The term “cancer” refers to a disease characterized by the rapid and uncontrolled growth of aberrant cells. Cancer cells can spread locally or through the bloodstream and lymphatic system to other parts of the body. As used herein, the term “cancer” or “tumor” includes premalignant, as well as malignant cancers and tumors.

As used herein, the terms “treat”, “treatment” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

Antibody Molecules

In one embodiment, the antibody molecule binds to a mammalian, e.g., human, PD-1. For example, the antibody molecule binds specifically to an epitope, e.g., linear or conformational epitope, (e.g., an epitope as described herein) on PD-1.

As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). In an embodiment, an antibody molecule comprises a full length antibody, or a full length immunoglobulin chain. In an embodiment, an antibody molecule comprises an antigen binding or functional fragment of a full length antibody, or a full length immunoglobulin chain. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

In an embodiment, an antibody molecule is a monospecific antibody molecule and binds a single epitope. E.g., a monospecific antibody molecule having a plurality of immunoglobulin variable domain sequences, each of which binds the same epitope.

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule,

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope. In an embodiment the first epitope is located on PD-1 and the second epitope is located on a TIM-3, LAG-3, CEACAM (e.g., CEACAM-1 and/or CEACAM-5), PD-L1, or PD-L2.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J Mol. Biol. 196:901-917; and the AbM definition used by Oxford Molecular's AbM antibody modeling software. See, generally, e.g., Protein Sequence and Structure Analysis ofAntibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273; 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Generally, unless specifically indicated, the anti-PD-1 antibody molecules can include any combination of one or more Kabat CDRs and/or Chothia hypervariable loops, e.g., described in Table 1. In one embodiment, the following definitions are used for the anti-PD-1 antibody molecules described in Table 1: HCDR1 according to the combined CDR definitions of both Kabat and Chothia, and HCCDRs 2-3 and LCCDRs 1-3 according the CDR definition of Kabat. Under all definitions, each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the PD-1 polypeptide, or an epitope thereof. With respect to proteins (or protein mimetics), the antigen-binding site typically includes one or more loops (of at least four amino acids or amino acid mimics) that form an interface that binds to the PD-1 polypeptide. Typically, the antigen-binding site of an antibody molecule includes at least one or two CDRs and/or hypervariable loops, or more typically at least three, four, five or six CDRs and/or hypervariable loops.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to PD-1. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor”. In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

Exemplary PD-1 Inhibitors

PD-1 is a CD28/CTLA-4 family member expressed, e.g., on activated CD4+ and CD8⁺ T cells, T_{regs}, and B cells. It negatively regulates effector T cell signaling and function. PD-1 is induced on tumor-infiltrating T cells, and can result in functional exhaustion or dysfunction (Keir et al. (2008) Annu. Rev. Immunol. 26:677-704; Pardoll et al. (2012) Nat Rev Cancer 12(4):252-64). PD-1 delivers a coinhibitory signal upon binding to either of its two ligands, Programmed Death-Ligand 1 (PD-L1) or Programmed Death-Ligand 2 (PD-L2). PD-L1 is expressed on a number of cell types, including T cells, natural killer (NK) cells, macrophages, dendritic cells (DCs), B cells, epithelial cells, vascular endothelial cells, as well as many types of tumors. High expression of PD-LI on murine and human tumors has been linked to poor clinical outcomes in a variety of cancers (Keir et al. (2008) Annu. Rev. Immunol. 26:677-704; Pardoll et al. (2012) Nat Rev Cancer 12(4):252-64). PD-L2 is expressed on dendritic cells, macrophages, and some tumors. Blockade of the PD-1 pathway has been pre-clinically and clinically validated for cancer immunotherapy. Both preclinical and clinical studies have demonstrated that anti-PD-1 blockade can restore activity of effector T cells and results in robust anti-tumor response. For example, blockade of PD-1 pathway can restore exhausted/dysfunctional effector T cell function (e.g., proliferation, IFN-γ secretion, or cytolytic function) and/or inhibit T_{reg} cell function (Keir et al. (2008) Annu. Rev. Immunol. 26:677-704; Pardoll et al. (2012) Nat Rev Cancer 12(4):252-64). Blockade of the PD-1 pathway can be effected with an antibody, an antigen binding fragment thereof, an immunoadhesin, a fusion protein, or oligopeptide of PD-1, PD-L1 and/or PD-L2.

As used herein, the term “Programmed Death 1” or “PD-1” include isoforms, mammalian, e.g., human PD-1, species homologs of human PD-1, and analogs comprising at least one common epitope with PD-1. The amino acid sequence of PD-1, e.g., human PD-1, is known in the art, e.g., Shinohara T et al. (1994) Genomics 23(3):704-6; Finger L R, et al. Gene (1997) 197(1-2):177-87.

The anti-PD-1 antibody molecules described herein can be used alone or in combination with one or more additional agents described herein in accordance with a method described herein. In certain embodiments, the combinations described herein include a PD-1 inhibitor, e.g., an anti-PD-1 antibody molecule (e.g., humanized antibody molecules) as described herein.

In one embodiment, the anti-PD-1 antibody molecule includes:

(a) a heavy chain variable region (VH) comprising a HCDR1 amino acid sequence of SEQ ID NO: 4, a HCDR2 amino acid sequence of SEQ ID NO: 5, and a HCDR3 amino acid sequence of SEQ ID NO: 3; and a light chain variable region (VL) comprising a LCDR1 amino acid sequence of SEQ ID NO: 13, a LCDR2 amino acid sequence of SEQ ID NO: 14, and a LCDR3 amino acid sequence of SEQ ID NO: 33;

(b) a VH comprising a HCDR1 amino acid sequence chosen from SEQ ID NO: 1; a HCDR2 amino acid sequence of SEQ ID NO: 2; and a HCDR3 amino acid sequence of SEQ ID NO: 3; and a VL comprising a LCDR1 amino acid sequence of SEQ ID NO: 10, a LCDR2 amino acid sequence of SEQ ID NO: 11, and a LCDR3 amino acid sequence of SEQ ID NO: 32;

(c) a VH comprising a HCDR1 amino acid sequence of SEQ ID NO: 4, a HCDR2 amino acid sequence of SEQ ID NO: 5, and a HCDR3 amino acid sequence of SEQ ID NO: 3; and a VL comprising a LCDR1 amino acid sequence of SEQ ID NO: 13, a LCDR2 amino acid sequence of SEQ ID NO: 14, and a LCDR3 amino acid sequence of SEQ ID NO: 33; or

(d) a VH comprising a HCDR1 amino acid sequence of SEQ ID NO: 1; a HCDR2 amino acid sequence of SEQ ID NO: 2; and a HCDR3 amino acid sequence of SEQ ID NO: 3; and a VL comprising a LCDR1 amino acid sequence of SEQ ID NO: 10, a LCDR2 amino acid sequence of SEQ ID NO: 11, and a LCDR3 amino acid sequence of SEQ ID NO: 32.

In one embodiment, the anti-PD-1 antibody molecule comprises:
(a) a heavy chain variable region (VH) comprising a HCDR1 amino acid sequence of SEQ ID NO: 4, a HCDR2 amino acid sequence of SEQ ID NO: 5, and a HCDR3 amino acid sequence of SEQ ID NO: 3; and a light chain variable region (VL) comprising a LCDR1 amino acid sequence of SEQ ID NO: 13, a LCDR2 amino acid sequence of SEQ ID NO: 14, and a LCDR3 amino acid sequence of SEQ ID NO: 33;
(b) a VH comprising a HCDR1 amino acid sequence of SEQ ID NO: 1; a HCDR2 amino acid sequence of SEQ ID NO: 2; and a HCDR3 amino acid sequence of SEQ ID NO: 3; and a VL comprising a LCDR1 amino acid sequence of SEQ ID NO: 10, a LCDR2 amino acid sequence of SEQ ID NO: 11, and a LCDR3 amino acid sequence of SEQ ID NO: 32;
(c) a VH comprising a HCDR1 amino acid sequence of SEQ ID NO: 224, a HCDR2 amino acid sequence of SEQ ID NO: 5, and a HCDR3 amino acid sequence of SEQ ID NO: 3; and a VL comprising a LCDR1 amino acid sequence of SEQ ID NO: 13, a LCDR2 amino acid sequence of SEQ ID NO: 14, and a LCDR3 amino acid sequence of SEQ ID NO: 33; or
(d) a VH comprising a HCDR1 amino acid sequence of SEQ ID NO: 224; a HCDR2 amino acid sequence of SEQ ID NO: 2; and a HCDR3 amino acid sequence of SEQ ID NO: 3; and a VL comprising a LCDR1 amino acid sequence of SEQ ID NO: 10, a LCDR2 amino acid sequence of SEQ ID NO: 11, and a LCDR3 amino acid sequence of SEQ ID NO: 32.

In certain embodiments, the anti-PD-1 antibody molecule comprises:

(i) a heavy chain variable region (VH) comprising a HCDR1 amino acid sequence chosen from SEQ ID NO: 1, SEQ ID NO: 4 or SEQ ID NO: 224; a HCDR2 amino acid sequence of SEQ ID NO: 2; and a HCDR3 amino acid sequence of SEQ ID NO: 3; and

(ii) a light chain variable region (VL) comprising a LCDR1 amino acid sequence of SEQ ID NO: 10, a LCDR2 amino acid sequence of SEQ ID NO: 11, and a LCDR3 amino acid sequence of SEQ ID NO: 32.

In other embodiments, the anti-PD-1 antibody molecule comprises:

(i) a heavy chain variable region (VH) comprising a HCDR1 amino acid sequence chosen from SEQ ID NO: 1, SEQ ID NO: 4 or SEQ ID NO: 224; a HCDR2 amino acid sequence of SEQ ID NO: 5, and a HCDR3 amino acid sequence of SEQ ID NO: 3; and

(ii) a light chain variable region (VL) comprising a LCDR1 amino acid sequence of SEQ ID NO: 13, a LCDR2 amino acid sequence of SEQ ID NO: 14, and a LCDR3 amino acid sequence of SEQ ID NO: 33.

In embodiments of the aforesaid antibody molecules, the HCDR1 comprises the amino acid sequence of SEQ ID NO: 1. In other embodiments, the HCDR1 comprises the amino acid sequence of SEQ ID NO: 4. In yet other embodiments, the HCDR1 amino acid sequence of SEQ ID NO: 224.

In embodiments, the aforesaid antibody molecules have a heavy chain variable region comprising at least one framework (FW) region comprising the amino acid sequence of any of SEQ ID NOs: 147, 151, 153, 157, 160, 162, 166, or 169, or an amino acid sequence at least 90% identical thereto, or having no more than two amino acid substitutions, insertions or deletions compared to the amino acid sequence of any of SEQ ID NOs: 147, 151, 153, 157, 160, 162, 166, or 169.

In other embodiments, the aforesaid antibody molecules have a heavy chain variable region comprising at least one framework region comprising the amino acid sequence of any of SEQ ID NOs: 147, 151, 153, 157, 160, 162, 166, or 169.

In yet other embodiments, the aforesaid antibody molecules have a heavy chain variable region comprising at least two, three, or four framework regions comprising the amino acid sequences of any of SEQ ID NOs: 147, 151, 153, 157, 160, 162, 166, or 169.

In other embodiments, the aforesaid antibody molecules comprise a VHFW1 amino acid sequence of SEQ ID NO: 147 or 151, a VHFW2 amino acid sequence of SEQ ID NO: 153, 157, or 160, and a VHFW3 amino acid sequence of SEQ ID NO: 162 or 166, and, optionally, further comprising a VHFW4 amino acid sequence of SEQ ID NO: 169.

In other embodiments, the aforesaid antibody molecules have a light chain variable region comprising at least one framework region comprising the amino acid sequence of any of SEQ ID NOs: 174, 177, 181, 183, 185, 187, 191, 194, 196, 200, 202, 205, or 208, or an amino acid sequence at least 90% identical thereto, or having no more than two amino acid substitutions, insertions or deletions compared to the amino acid sequence of any of 174, 177, 181, 183, 185, 187, 191, 194, 196,200,202,205, or 208.

In other embodiments, the aforesaid antibody molecules have a light chain variable region comprising at least one framework region comprising the amino acid sequence of any of SEQ ID NOs: 174, 177, 181, 183, 185, 187, 191, 194, 196, 200, 202, 205, or 208.

In other embodiments, the aforesaid antibody molecules have a light chain variable region comprising at least two, three, or four framework regions comprising the amino acid sequences of any of SEQ ID NOs: 174, 177, 181, 183, 185, 187, 191, 194, 196, 200, 202, 205, or 208.

In other embodiments, the aforesaid antibody molecules comprise a VLFW1 amino acid sequence of SEQ ID NO: 174, 177, 181, 183, or 185, a VLFW2 amino acid sequence of SEQ ID NO: 187, 191, or 194, and a VLFW3 amino acid sequence of SEQ ID NO: 196, 200, 202, or 205, and, optionally, further comprising a VLFW4 amino acid sequence of SEQ ID NO: 208.

In other embodiments, the aforesaid antibodies comprise a heavy chain variable domain comprising an amino acid sequence at least 85% identical to any of SEQ ID NOs: 38, 50, 82, or 86.

In other embodiments, the aforesaid antibody molecules comprise a heavy chain variable domain comprising the amino acid sequence of SEQ ID NO: 38, 50, 82, or 86.

In other embodiments, the aforesaid antibody molecules comprise a light chain variable domain comprising an amino acid sequence at least 85% identical to any of SEQ ID NOs: 42, 46, 54, 58, 62, 66, 70, 74, or 78.

In other embodiments, the aforesaid antibody molecules comprise a light chain variable domain comprising the amino acid sequence of SEQ ID NO: 42, 46, 54, 58, 62, 66, 70, 74, or 78.

In other embodiments, the aforesaid antibody molecules comprise a heavy chain variable domain comprising the amino acid sequence of SEQ ID NO: 38.

In other embodiments, the aforesaid antibody molecules comprise a heavy chain comprising the amino acid sequence of SEQ ID NO: 40.

In other embodiments, the aforesaid antibody molecules comprise a heavy chain comprising the amino acid sequence of SEQ ID NO: 91.

In other embodiments, the aforesaid antibody molecules comprise a heavy chain variable domain comprising the amino acid sequence of SEQ ID NO: 50.

In other embodiments, the aforesaid antibody molecules comprise a heavy chain comprising the amino acid sequence of SEQ ID NO: 52 or SEQ ID NO: 102.

In other embodiments, the aforesaid antibody molecules comprise a heavy chain variable domain comprising the amino acid sequence of SEQ ID NO: 82.

In other embodiments, the aforesaid antibody molecules comprise a heavy chain comprising the amino acid sequence of SEQ ID NO: 84.

In other embodiments, the aforesaid antibody molecules comprise a heavy chain variable domain comprising the amino acid sequence of SEQ ID NO: 86.

In other embodiments, the aforesaid antibody molecules comprise a heavy chain comprising the amino acid sequence of SEQ ID NO: 88.

In other embodiments, the aforesaid antibody molecules comprise a light chain variable domain comprising the amino acid sequence of SEQ ID NO: 42.

In other embodiments, the aforesaid antibody molecules comprise a light chain comprising the amino acid sequence of SEQ ID NO: 44.

In other embodiments, the aforesaid antibody molecules comprise a light chain variable domain comprising the amino acid sequence of SEQ ID NO: 46.

In other embodiments, the aforesaid antibody molecules comprise a light chain comprising the amino acid sequence of SEQ ID NO: 48.

In other embodiments, the aforesaid antibody molecules comprise a light chain variable domain comprising the amino acid sequence of SEQ ID NO: 54.

In other embodiments, the aforesaid antibody molecules comprise a light chain comprising the amino acid sequence of SEQ ID NO: 56.

In other embodiments, the aforesaid antibody molecules comprise a light chain variable domain comprising the amino acid sequence of SEQ ID NO: 58.

In other embodiments, the aforesaid antibody molecules comprise a light chain comprising the amino acid sequence of SEQ ID NO: 60.

In other embodiments, the aforesaid antibody molecules comprise a light chain variable domain comprising the amino acid sequence of SEQ ID NO: 62.

In other embodiments, the aforesaid antibodies comprise a light chain comprising the amino acid sequence of SEQ ID NO: 64.

In other embodiments, the aforesaid antibody molecules comprise a light chain variable domain comprising the amino acid sequence of SEQ ID NO: 66.

In other embodiments, the aforesaid antibody molecules comprise a light chain comprising the amino acid sequence of SEQ ID NO: 68.

In other embodiments, the aforesaid antibody molecules comprise a light chain variable domain comprising the amino acid sequence of SEQ ID NO: 70.

In other embodiments, the aforesaid antibody molecules comprise a light chain comprising the amino acid sequence of SEQ ID NO: 72.

In other embodiments, the aforesaid antibody molecules comprise a light chain variable domain comprising the amino acid sequence of SEQ ID NO: 74.

In other embodiments, the aforesaid antibody molecules comprise a light chain comprising the amino acid sequence of SEQ ID NO: 76.

In other embodiments, the aforesaid antibody molecules comprise a light chain variable domain comprising the amino acid sequence of SEQ ID NO: 78.

In other embodiments, the aforesaid antibody molecules comprise a light chain comprising the amino acid sequence of SEQ ID NO: 80.

In other embodiments, the aforesaid antibody molecules comprise a heavy chain variable domain comprising the amino acid sequence of SEQ ID NO: 38 and a light chain variable domain comprising the amino acid sequence of SEQ ID NO: 42.

In other embodiments, the aforesaid antibody molecules comprise a heavy chain variable domain comprising the amino acid sequence of SEQ ID NO: 38 and a light chain variable domain comprising the amino acid sequence of SEQ ID NO: 66.

In other embodiments, the aforesaid antibody molecules comprise a heavy chain variable domain comprising the amino acid sequence of SEQ ID NO: 38 and a light chain variable domain comprising the amino acid sequence of SEQ ID NO: 70.

In other embodiments, the aforesaid antibody molecules comprise a heavy chain variable domain comprising the amino acid sequence of SEQ ID NO: 50 and a light chain variable domain comprising the amino acid sequence of SEQ ID NO: 70.

In other embodiments, the aforesaid antibody molecules comprise a heavy chain variable domain comprising the amino acid sequence of SEQ ID NO: 38 and a light chain variable domain comprising the amino acid sequence of SEQ ID NO: 46.

In other embodiments, the aforesaid antibody molecules comprise a heavy chain variable domain comprising the amino acid sequence of SEQ ID NO: 50 and a light chain variable domain comprising the amino acid sequence of SEQ ID NO: 46.

In other embodiments, the aforesaid antibody molecules comprise a heavy chain variable domain comprising the amino acid sequence of SEQ ID NO: 50 and a light chain variable domain comprising the amino acid sequence of SEQ ID NO: 54.

In other embodiments, the aforesaid antibody molecules comprise a heavy chain variable domain comprising the amino acid sequence of SEQ ID NO: 38 and a light chain variable domain comprising the amino acid sequence of SEQ ID NO: 54.

In other embodiments, the aforesaid antibody molecules comprise a heavy chain variable domain comprising the amino acid sequence of SEQ ID NO: 38 and a light chain variable domain comprising the amino acid sequence of SEQ ID NO: 58.

In other embodiments, the aforesaid antibody molecules comprise a heavy chain variable domain comprising the amino acid sequence of SEQ ID NO: 38 and a light chain variable domain comprising the amino acid sequence of SEQ ID NO: 62.

In other embodiments, the aforesaid antibody molecules comprise a heavy chain variable domain comprising the amino acid sequence of SEQ ID NO: 50 and a light chain variable domain comprising the amino acid sequence of SEQ ID NO: 66.

In other embodiments, the aforesaid antibody molecules comprise a heavy chain variable domain comprising the amino acid sequence of SEQ ID NO: 38 and a light chain variable domain comprising the amino acid sequence of SEQ ID NO: 74.

In other embodiments, the aforesaid antibody molecules comprise a heavy chain variable domain comprising the amino acid sequence of SEQ ID NO: 38 and a light chain variable domain comprising the amino acid sequence of SEQ ID NO: 78.

In other embodiments, the aforesaid antibody molecules comprise a heavy chain variable domain comprising the amino acid sequence of SEQ ID NO: 82 and a light chain variable domain comprising the amino acid sequence of SEQ ID NO: 70.

In other embodiments, the aforesaid antibody molecules comprise a heavy chain variable domain comprising the amino acid sequence of SEQ ID NO: 82 and a light chain variable domain comprising the amino acid sequence of SEQ ID NO: 66.

In other embodiments, the aforesaid antibody molecules comprise a heavy chain variable domain comprising the amino acid sequence of SEQ ID NO: 86 and a light chain variable domain comprising the amino acid sequence of SEQ ID NO: 66.

In other embodiments, the aforesaid antibody molecules comprise a heavy chain comprising the amino acid sequence of SEQ ID NO: 91 and a light chain comprising the amino acid sequence of SEQ ID NO: 44.

In other embodiments, the aforesaid antibody molecules comprise a heavy chain comprising the amino acid sequence of SEQ ID NO: 91 and a light chain comprising the amino acid sequence of SEQ ID NO: 56.

In other embodiments, the aforesaid antibody molecules comprise a heavy chain comprising the amino acid sequence of SEQ ID NO: 91 and a light chain comprising the amino acid sequence of SEQ ID NO: 68.

In other embodiments, the aforesaid antibody molecules comprise a heavy chain comprising the amino acid sequence of SEQ ID NO: 91 and a light chain comprising the amino acid sequence of SEQ ID NO: 72.

In other embodiments, the aforesaid antibody molecules comprise a heavy chain comprising the amino acid sequence of SEQ ID NO: 102 and a light chain comprising the amino acid sequence of SEQ ID NO: 72.

In other embodiments, the aforesaid antibody molecules comprise a heavy chain comprising the amino acid sequence of SEQ ID NO: 40 and a light chain comprising the amino acid sequence of SEQ ID NO: 44.

In other embodiments, the aforesaid antibody molecules comprise a heavy chain comprising the amino acid sequence of SEQ ID NO: 40 and a light chain comprising the amino acid sequence of SEQ ID NO: 48.

In other embodiments, the aforesaid antibody molecules comprise a heavy chain comprising the amino acid sequence of SEQ ID NO: 52 and a light chain comprising the amino acid sequence of SEQ ID NO: 48.

In other embodiments, the aforesaid antibody molecules comprise a heavy chain comprising the amino acid sequence of SEQ ID NO: 52 and a light chain comprising the amino acid sequence of SEQ ID NO: 56.

In other embodiments, the aforesaid antibody molecules comprise a heavy chain comprising the amino acid sequence of SEQ ID NO: 40 and a light chain comprising the amino acid sequence of SEQ ID NO: 56.

In other embodiments, the aforesaid antibodies comprise a heavy chain comprising the amino acid sequence of SEQ ID NO: 40 and a light chain comprising the amino acid sequence of SEQ ID NO: 60.

In other embodiments, the aforesaid antibody molecules comprise a heavy chain comprising the amino acid sequence of SEQ ID NO: 40 and a light chain comprising the amino acid sequence of SEQ ID NO: 64.

In other embodiments, the aforesaid antibody molecules comprise a heavy chain comprising the amino acid sequence of SEQ ID NO: 52 and a light chain comprising the amino acid sequence of SEQ ID NO: 68.

In other embodiments, the aforesaid antibody molecules comprise a heavy chain comprising the amino acid sequence of SEQ ID NO: 40 and a light chain comprising the amino acid sequence of SEQ ID NO: 68.

In other embodiments, the aforesaid antibody molecules comprise a heavy chain comprising the amino acid sequence of SEQ ID NO: 52 and a light chain comprising the amino acid sequence of SEQ ID NO: 72.

In other embodiments, the aforesaid antibody molecules comprise a heavy chain comprising the amino acid sequence of SEQ ID NO: 40 and a light chain comprising the amino acid sequence of SEQ ID NO: 72.

In other embodiments, the aforesaid antibody molecules comprise a heavy chain comprising the amino acid sequence of SEQ ID NO: 40 and a light chain comprising the amino acid sequence of SEQ ID NO: 76.

In other embodiments, the aforesaid antibody molecules comprise a heavy chain comprising the amino acid sequence of SEQ ID NO: 40 and a light chain comprising the amino acid sequence of SEQ ID NO: 80.

In other embodiments, the aforesaid antibody molecules comprise a heavy chain comprising the amino acid sequence of SEQ ID NO: 84 and a light chain comprising the amino acid sequence of SEQ ID NO: 72.

In other embodiments, the aforesaid antibodies comprise a heavy chain comprising the amino acid sequence of SEQ ID NO: 84 and a light chain comprising the amino acid sequence of SEQ ID NO: 68.

In other embodiments, the aforesaid antibody molecules comprise a heavy chain comprising the amino acid sequence of SEQ ID NO: 88 and a light chain comprising the amino acid sequence of SEQ ID NO: 68.

In other embodiments, the aforesaid antibody molecules are chosen from a Fab, F(ab′)2, Fv, or a single chain Fv fragment (scFv).

In other embodiments, the aforesaid antibody molecules comprise a heavy chain constant region selected from IgG1, IgG2, IgG3, and IgG4.

In other embodiments, the aforesaid antibody molecules comprise a light chain constant region chosen from the light chain constant regions of kappa or lambda.

In other embodiments, the aforesaid antibody molecules comprise a human IgG4 heavy chain constant region with a mutation at position 228 according to EU numbering or position 108 of SEQ ID NO: 212 or 214 and a kappa light chain constant region.

In other embodiments, the aforesaid antibody molecules comprise a human IgG4 heavy chain constant region with a Serine to Proline mutation at position 228 according to EU numbering or position 108 of SEQ ID NO: 212 or 214 and a kappa light chain constant region.

In other embodiments, the aforesaid antibody molecules comprise a human IgG1 heavy chain constant region with an Asparagine to Alanine mutation at position 297 according to EU numbering or position 180 of SEQ ID NO: 216 and a kappa light chain constant region.

In other embodiments, the aforesaid antibody molecules comprise a human IgG1 heavy chain constant region with an Aspartate to Alanine mutation at position 265 according to EU numbering or position 148 of SEQ ID NO: 217, and Proline to Alanine mutation at position 329 according to EU numbering or position 212 of SEQ ID NO: 217 and a kappa light chain constant region.

In other embodiments, the aforesaid antibody molecules comprise a human IgG1 heavy chain constant region with a Leucine to Alanine mutation at position 234 according to EU numbering or position 117 of SEQ ID NO: 218, and Leucine to Alanine mutation at position 235 according to EU numbering or position 118 of SEQ ID NO: 218 and a kappa light chain constant region.

In other embodiments, the aforesaid antibody molecules are capable of binding to human PD-1 with a dissociation constant (K_{D}) of less than about 0.2 nM.

In some embodiments, the aforesaid antibody molecules bind to human PD-1 with a K_{D} of less than about 0.2 nM, 0.15 nM, 0.1 nM, 0.05 nM, or 0.02 nM, e.g., about 0.13 nM to 0.03 nM, e.g., about 0.077 nM to 0.088 nM, e.g., about 0.083 nM, e.g., as measured by a Biacore method.

In other embodiments, the aforesaid antibody molecules bind to cynomolgus PD-1 with a K_{D} of less than about 0.2 nM, 0.15 nM, 0.1 nM, 0.05 nM, or 0.02 nM, e.g., about 0.11 nM to 0.08 nM, e.g., about 0.093 nM, e.g., as measured by a Biacore method.

In certain embodiments, the aforesaid antibody molecules bind to both human PD-1 and cynomolgus PD-1 with similar K_{D}, e.g., in the nM range, e.g., as measured by a Biacore method. In some embodiments, the aforesaid antibody molecules bind to a human PD-1-Ig fusion protein with a K_{D} of less than about 0.1 nM, 0.075 nM, 0.05 nM, 0.025 nM, or 0.01 nM, e.g., about 0.04 nM, e.g., as measured by ELISA.

In some embodiments, the aforesaid antibody molecules bind to Jurkat cells that express human PD-1 (e.g., human PD-1-transfected Jurkat cells) with a K_{D} of less than about 0.1 nM, 0.075 nM, 0.05 nM, 0.025 nM, or 0.01 nM, e.g., about 0.06 nM, e.g., as measured by FACS analysis.

In some embodiments, the aforesaid antibody molecules bind to cynomolgus T cells with a K_{D} of less than about 1 nM, 0.75 nM, 0.5 nM, 0.25 nM, or 0.1 nM, e.g., about 0.4 nM, e.g., as measured by FACS analysis.

In some embodiments, the aforesaid antibody molecules bind to cells that express cynomolgus PD-1 (e.g., cells transfected with cynomolgus PD-1) with a K_{D} of less than about 1 nM, 0.75 nM, 0.5 nM, 0.25 nM, or 0.01 nM, e.g., about 0.6 nM, e.g., as measured by FACS analysis.

In certain embodiments, the aforesaid antibody molecules are not cross-reactive with mouse or rat PD-1. In other embodiments, the aforesaid antibodies are cross-reactive with rhesus PD-1. For example, the cross-reactivity can be measured by a Biacore method or a binding assay using cells that expresses PD-1 (e.g., human PD-1-expressing 300.19 cells). In other embodiments, the aforesaid antibody molecules bind an extracellular Ig-like domain of PD-1.

In other embodiments, the aforesaid antibody molecules are capable of reducing binding of PD-1 to PD-L1, PD-L2, or both, or a cell that expresses PD-L1, PD-L2, or both.

In some embodiments, the aforesaid antibody molecules reduce (e.g., block) PD-L1 binding to a cell that expresses PD-1 (e.g., human PD-1-expressing 300.19 cells) with an IC50 of less than about 1.5 nM, 1 nM, 0.8 nM, 0.6 nM, 0.4 nM, 0.2 nM, or 0.1 nM, e.g., between about 0.79 nM and about 1.09 nM, e.g., about 0.94 nM, or about 0.78 nM or less, e.g., about 0.3 nM. In some embodiments, the aforesaid antibodies reduce (e.g., block) PD-L2 binding to a cell that expresses PD-1 (e.g., human PD-1-expressing 300.19 cells) with an IC50 of less than about 2 nM, 1.5 nM, 1 nM, 0.5 nM, or 0.2 nM, e.g., between about 1.05 nM and about 1.55 nM, or about 1.3 nM or less, e.g., about 0.9 nM.

In other embodiments, the aforesaid antibody molecules are capable of enhancing an antigen-specific T cell response.

In embodiments, the antibody molecule is a monospecific antibody molecule or a bispecific antibody molecule. In embodiments, the antibody molecule has a first binding specificity for PD-1 and a second binding specify for TIM-3, LAG-3, CEACAM (e.g., CEACAM-1, CEACAM-3, and/or CEACAM-5), PD-L1 or PD-L2. In embodiments, the antibody molecule comprises an antigen binding fragment of an antibody, e.g., a half antibody or antigen binding fragment of a half antibody.

In some embodiments, the aforesaid antibody molecules increase the expression of TL-2 from cells activated by Staphylococcal enterotoxin B (SEB) (e.g., at 25 μg/mL) by at least about 2, 3, 4, 5-fold, e.g., about 2 to 3-fold, e.g., about 2 to 2.6-fold, e.g., about 2.3-fold, compared to the expression of IL-2 when an isotype control (e.g., IgG4) is used, e.g., as measured in a SEB T cell activation assay or a human whole blood ex vivo assay.

In some embodiments, the aforesaid antibody molecules increase the expression of IFN-γ from T cells stimulated by anti-CD3 (e.g., at 0.1 μg/mL) by at least about 2, 3, 4, 5-fold, e.g., about 1.2 to 3.4-fold, e.g., about 2.3-fold, compared to the expression of IFN-γ when an isotype control (e.g., IgG4) is used, e.g., as measured in an IFN-γ activity assay.

In some embodiments, the aforesaid antibody molecules increase the expression of IFN-γ from T cells activated by SEB (e.g., at 3 pg/mL) by at least about 2, 3, 4, 5-fold, e.g., about 0.5 to 4.5-fold, e.g., about 2.5-fold, compared to the expression of IFN-γ when an isotype control (e.g., IgG4) is used, e.g., as measured in an IFN-γ activity assay.

In some embodiments, the aforesaid antibody molecules increase the expression of IFN-γ from T cells activated with an CMV peptide by at least about 2, 3, 4, 5-fold, e.g., about 2 to 3.6-fold, e.g., about 2.8-fold, compared to the expression of IFN-γ when an isotype control (e.g., IgG4) is used, e.g., as measured in an IFN-γ activity assay.

In some embodiments, the aforesaid antibody molecules increase the proliferation of CD8⁺ T cells activated with an CMV peptide by at least about 1, 2, 3, 4, 5-fold, e.g., about 1.5-fold, compared to the proliferation of CD8⁺ T cells when an isotype control (e.g., IgG4) is used, e.g., as measured by the percentage of CD8⁺ T cells that passed through at least n (e.g., n=2 or 4) cell divisions.

In certain embodiments, the aforesaid antibody molecules has a Cmax between about 100 μg/mL and about 500 μg/mL, between about 150 μg/mL and about 450 μg/mL, between about 250 μg/mL and about 350 μg/mL, or between about 200 μg/mL and about 400 μg/mL, e.g., about 292.5 μg/mL, e.g., as measured in monkey.

In certain embodiments, the aforesaid antibody molecules has a T₁ between about 250 hours and about 650 hours, between about 300 hours and about 600 hours, between about 350 hours and about 550 hours, or between about 400 hours and about 500 hours, e.g., about 465.5 hours, e.g., as measured in monkey.

In some embodiments, the aforesaid antibody molecules bind to PD-1 with a Kd slower than 5×10⁻⁴, 1×10⁻⁴, 5×10⁻⁵, or 1×10⁻⁵ s⁻¹, e.g., about 2.13×10⁻⁴ s⁻¹, e.g., as measured by a Biacore method. In some embodiments, the aforesaid antibody molecules bind to PD-1 with a Ka faster than 1×10⁴, 5×10⁴, 1×10⁵, or 5×10⁵ M⁻¹s⁻¹, e.g., about 2.78×10⁵ M⁻¹s⁻¹, e.g., as measured by a Biacore method.

In some embodiments, the aforesaid anti-PD-1 antibody molecules bind to one or more residues within the C strand, CC′ loop, C′ strand and FG loop of PD-1. The domain structure of PD-1 is described, e.g., in Cheng et al., “Structure and Interactions of the Human Programmed Cell Death 1 Receptor” J Biol. Chem. 2013, 288:11771-11785. As described in Cheng et. al., the C strand comprises residues F43-M50, the CC′ loop comprises S51-N54, the C′ strand comprises residues Q55-F62, and the FG loop comprises residues L108-I114 (amino acid numbering according to Chang et al. supra). Accordingly, in some embodiments, an anti-PD-1 antibody as described herein binds to at least one residue in one or more of the ranges F43-M50, S51-N54, Q55-F62, and L108-I114 of PD-1. In some embodiments, an anti-PD-1 antibody as described herein binds to at least one residue in two, three, or all four of the ranges F43-M50, S51-N54, Q55-F62, and L108-I114 of PD-1. In some embodiments, the anti-PD-1 antibody binds to a residue in PD-1 that is also part of a binding site for one or both of PD-LI and PD-L2.

In another aspect, the invention provides an isolated nucleic acid molecule encoding any of the aforesaid antibody molecules, vectors and host cells thereof.

An isolated nucleic acid encoding the antibody heavy chain variable region or light chain variable region, or both, of any the aforesaid antibody molecules is also provided.

In one embodiment, the isolated nucleic acid encodes heavy chain CDRs 1-3, wherein said nucleic acid comprises a nucleotide sequence of SEQ ID NO: 108-112, 223, 122-126, 133-137, or 144-146.

In another embodiment, the isolated nucleic acid encodes light chain CDRs 1-3, wherein said nucleic acid comprises a nucleotide sequence of SEQ ID NO: 113-120, 127-132, or 138-143.

In other embodiments, the aforesaid nucleic acid further comprises a nucleotide sequence encoding a heavy chain variable domain, wherein said nucleotide sequence is at least 85% identical to any of SEQ ID NO: 39, 51, 83, 87, 90, 95, or 101.

In other embodiments, the aforesaid nucleic acid further comprises a nucleotide sequence encoding a heavy chain variable domain, wherein said nucleotide sequence comprises any of SEQ ID NO: 39, 51, 83, 87, 90, 95, or 101.

In other embodiments, the aforesaid nucleic acid further comprises a nucleotide sequence encoding a heavy chain, wherein said nucleotide sequence is at least 85% identical to any of SEQ ID NO: 41, 53, 85, 89, 92, 96, or 103.

In other embodiments, the aforesaid nucleic acid further comprises a nucleotide sequence encoding a heavy chain, wherein said nucleotide sequence comprises any of SEQ ID NO: 41, 53, 85, 89, 92, 96, or 103.

In other embodiments, the aforesaid nucleic acid further comprises a nucleotide sequence encoding a light chain variable domain, wherein said nucleotide sequence is at least 85% identical to any of SEQ ID NO: 45, 49, 57, 61, 65, 69, 73, 77, 81, 94, 98, 100, 105, or 107.

In other embodiments, the aforesaid nucleic acid further comprises a nucleotide sequence encoding a light chain variable domain, wherein said nucleotide sequence comprises any of SEQ ID NO: 45, 49, 57, 61, 65, 69, 73, 77, 81, 94, 98, 100, 105, or 107.

In other embodiments, the aforesaid nucleic acid further comprises a nucleotide sequence encoding a light chain, wherein said nucleotide sequence is at least 85% identical to any of SEQ ID NO: 45, 49, 57, 61, 65, 69, 73, 77, 81, 94, 98, 100, 105 or 107.

In other embodiments, the aforesaid nucleic acid further comprises a nucleotide sequence encoding a light chain, wherein said nucleotide sequence comprises any of SEQ ID NO: 45, 49, 57, 61, 65, 69, 73, 77, 81, 94, 98, 100, 105 or 107.

In certain embodiments, one or more expression vectors and host cells comprising the aforesaid nucleic acids are provided.

A method of producing an antibody molecule or fragment thereof, comprising culturing the host cell as described herein under conditions suitable for gene expression is also provided.

In one aspect, the invention features a method of providing an antibody molecule described herein. The method includes: providing a PD-1 antigen (e.g., an antigen comprising at least a portion of a PD-1 epitope); obtaining an antibody molecule that specifically binds to the PD-1 polypeptide; and evaluating if the antibody molecule specifically binds to the PD-1 polypeptide, or evaluating efficacy of the antibody molecule in modulating, e.g., inhibiting, the activity of the PD-1. The method can further include administering the antibody molecule to a subject, e.g., a human or non-human animal.

In another aspect, the invention provides, compositions, e.g., pharmaceutical compositions, which include a pharmaceutically acceptable carrier, excipient or stabilizer, and at least one of the therapeutic agents, e.g., anti-PD-1 antibody molecules described herein. In one embodiment, the composition, e.g., the pharmaceutical composition, includes a combination of the antibody molecule and one or more agents, e.g., a therapeutic agent or other antibody molecule, as described herein. In one embodiment, the antibody molecule is conjugated to a label or a therapeutic agent.

In certain embodiments, the combinations described herein comprises a PD-1 inhibitor which is chosen from Spartalizumab (PDR001, Novartis), Nivolumab (Bristol-Myers Squibb), Pembrolizumab (Merck & Co), Pidilizumab (CureTech), MEDIO680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

Pharmaceutical Compositions and Kits

In another aspect, the present invention provides compositions, e.g., pharmaceutically acceptable compositions, which include an antibody molecule described herein, formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions of this invention may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody molecule can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody molecule is 0.1-30 mg/kg, more preferably 1-25 mg/kg. Dosages and therapeutic regimens of the anti-PD-1 antibody molecule can be determined by a skilled artisan. In certain embodiments, the anti-PD-1 antibody molecule is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the anti-PD-1 antibody molecule is administered at a dose from about 10 to 20 mg/kg every other week.

As another example, non-limiting range for a therapeutically or prophylactically effective amount of an antibody molecule is 200-500 mg, more preferably 300-400 mg/kg. Dosages and therapeutic regimens of the anti-PD-1 antibody molecule can be determined by a skilled artisan. In certain embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. The dosing schedule (e.g., flat dosing schedule) can vary from e.g., once a week to once every 2, 3, 4, 5, or 6 weeks. In one embodiment the anti-PD-1 antibody molecule is administered at a dose from about 300 mg to 400 mg once every three or once every four weeks. In one embodiment, the anti-PD-1 antibody molecule is administered at a dose from about 300 mg once every three weeks. In one embodiment, the anti-PD-1 antibody molecule is administered at a dose from about 400 mg once every four weeks. In one embodiment, the anti-PD-1 antibody molecule is administered at a dose from about 300 mg once every four weeks. In one embodiment, the anti-PD-1 antibody molecule is administered at a dose from about 400 mg once every three weeks. While not wishing to be bound by theory, in some embodiments, flat or fixed dosing can be beneficial to patients, for example, to save drug supply and to reduce pharmacy errors.

In some embodiments, the clearance (CL) of the anti-PD-1 antibody molecule is from about 6 to 16 mL/h, e.g., about 7 to 15 mL/h, about 8 to 14 mL/h, about 9 to 12 mL/h, or about 10 to 11 mL/h, e.g., about 8.9 mL/h, 10.9 mL/h, or 13.2 mL/h.

In some embodiments, the exponent of weight on CL of the anti-PD-1 antibody molecule is from about 0.4 to 0.7, about 0.5 to 0.6, or 0.7 or less, e.g., 0.6 or less, or about 0.54.

In some embodiments, the volume of distribution at steady state (Vss) of the anti-PD-1 antibody molecule is from about 5 to 10 V, e.g., about 6 to 9 V, about 7 to 8 V, or about 6.5 to 7.5 V, e.g., about 7.2 V.

In some embodiments, the half-life of the anti-PD-1 antibody molecule is from about 10 to 30 days, e.g., about 15 to 25 days, about 17 to 22 days, about 19 to 24 days, or about 18 to 22 days, e.g., about 20 days.

In some embodiments, the Cmin (e.g., for a 80 kg patient) of the anti-PD-1 antibody molecule is at least about 0.4 μg/mL, e.g., at least about 3.6 μg/mL, e.g., from about 20 to 50 μg/mL, e.g., about 22 to 42 μg/mL, about 26 to 47 μg/mL, about 22 to 26 μg/mL, about 42 to 47 μg/mL, about 25 to 35 μg/mL, about 32 to 38 μg/mL, e.g., about 31 μg/mL or about 35 μg/mL. In one embodiment, the Cmin is determined in a patient receiving the anti-PD-1 antibody molecule at a dose of about 400 mg once every four weeks. In another embodiment, the Cmin is determined in a patient receiving the anti-PD-1 antibody molecule at a dose of about 300 mg once every three weeks. In certain embodiments, the Cmin is at least about 50-fold higher, e.g., at least about 60-fold, 65-fold, 70-fold, 75-fold, 80-fold, 85-fold, 90-fold, 95-fold, or 100-fold, e.g., at least about 77-fold, higher than the EC50 of the anti-PD-1 antibody molecule, e.g., as determined based on IL-2 change in an SEB ex-vivo assay. In other embodiments, the Cmin is at least 5-fold higher, e.g., at least 6-fold, 7-fold, 8-fold, 9-fold, or 10-fold, e.g., at least about 8.6-fold, higher than the EC90 of the anti-PD-1 antibody molecule, e.g., as determined based on IL-2 change in an SEB ex-vivo assay.

The antibody molecule can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the modified antibody or antibody fragment is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention is a kit comprising an antibody molecule described herein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

Further Uses of the Combination Therapies

The combinations, e.g., the anti-PD-1 antibody molecules disclosed herein, have in vitro and in vivo diagnostic, as well as therapeutic and prophylactic utilities. For example, these molecules can be administered to cells in culture, in vitro or ex vivo, or to a human subject, to treat, prevent, and/or diagnose a variety of disorders, such as cancers and infectious disorders.

Accordingly, in one aspect, the invention provides a method of modifying an immune response in a subject comprising administering to the subject the combination described herein, such that the immune response in the subject is modified. In one embodiment, the immune response is enhanced, stimulated or up-regulated.

As used herein, the term “subject” is a human patient having a disorder or condition characterized by abnormal PD-1 functioning.

Throughout the text of this application, should there be a discrepancy between the text of the specification and the sequence listing, the text of the specification shall prevail.

TABLE 1
Amino acid and nucleotide sequences for murine, chimeric and humanized antibody
molecules. The antibody molecules include murine mAb BAP049, chimeric mAbs BAP049-
chi and BAP049-chi-Y, and humanized mAbs BAP049-hum01 to BAP049-hum16 and
BAP049-Clone-A to BAP049-Clone-E. The amino acid and nucleotide sequences of the
heavy and light chain CDRs, the heavy and light chain variable regions, and the
heavy and light chains are shown.

| BAP049 HC | | |
|---|---|---|
| SEQ ID NO: 1 (Kabat) | HCDR1 | TYWMH |
| SEQ ID NO: 2 (Kabat) | HCDR2 | NTYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 6 | VH | QVQLQQPGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTLVTVSA |
| SEQ ID NO: 7 | DNA VH | CAGGTCCAGCTGCAGCAACCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAAGGG |
| | | ACTCTGGTCACTGTCTCTGCA |
| SEQ ID NO: 8 | VH | QVQLQQSGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | LTTGTGAYWGQGTLVTVSA |
| SEQ ID NO: 9 | DNA VH | CAGGTCCAGCTGCAGCAGTCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAAGGG |
| | | ACTCTGGTCACTGTCTCTGCA |
| BAP049 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 12 (Kabat) | LCDR3 | QNDYSYPCT |
| SEQ ID NO: 13 (Chothia) | LCDR1 | SQSLLDSGNQKNF |
| SEQ ID NO: 14 (Chothia) | LCDR2 | WAS |
| SEQ ID NO: 15 (Chothia) | LCDR3 | DYSYPC |
| SEQ ID NO: 16 | VL | DIVMTQSPSSLTVTAGEKVTMSCKSSQSLLDSG |
| | | NQKNFLTWYQQKPGQPPKLLIFWASTRESGVPD |
| | | RFTGSGSVTDFTLTISSVQAEDLAVYYCQNDYS |
| | | YPCTFGGGTKLEIK |
| SEQ ID NO: 17 | DNA VL | GACATTGTGATGACCCAGTCTCCATCCTCCCTG |
| | | ACTGTGACAGCAGGAGAGAAGGTCACTATGAGC |
| | | TGCAAGTCCAGTCAGAGTCTGTTAGACAGTGGA |
| | | AATCAAAAGAACTTCTTGACCTGGTACCAGCAG |
| | | AAACCAGGGCAGCCTCCTAAACTGTTGATCTTC |
| | | TGGGCATCCACTAGGGAATCTGGGGTCCCTGAT |
| | | CGCTTCACAGGCAGTGGATCTGTAACAGATTTC |
| | | ACTCTCACCATCAGCAGTGTGCAGGCTGAAGAC |
| | | CTGGCAGTTTATTACTGTCAGAATGATTATAGT |
| | | TATCCGTGCACGTTCGGAGGGGGGACCAAGCTG |
| | | GAAATAAAA |
| BAP049-chi HC | | |
| SEQ ID NO: 1 (Kabat) | HCDR1 | TYWMH |
| SEQ ID NO: 2 (Kabat) | HCDR2 | NTYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 18 | VH | QVQLQQPGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSS |
| SEQ ID NO: 19 | DNA VH | CAGGTCCAGCTGCAGCAGCCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCC |
| SEQ ID NO: 20 | HC | QVQLQQPGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 21 | DNA HC | CAGGTCCAGCTGCAGCAGCCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| SEQ ID NO: 22 | VH | QVQLQQSGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSS |
| SEQ ID NO: 23 | DNA VH | CAGGTCCAGCTGCAGCAGTCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCC |
| SEQ ID NO: 30 | HC | QVQLQQSGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 31 | DNA HC | CAGGTCCAGCTGCAGCAGTCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-chi LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 12 (Kabat) | LCDR3 | QNDYSYPCT |
| SEQ ID NO: 13 (Chothia) | LCDR1 | SQSLLDSGNQKNF |
| SEQ ID NO: 14 (Chothia) | LCDR2 | WAS |
| SEQ ID NO: 15 (Chothia) | LCDR3 | DYSYPC |
| SEQ ID NO: 24 | VL | DIVMTQSPSSLTVTAGEKVTMSCKSSQSLLDSG |
| | | NQKNFLTWYQQKPGQPPKLLIFWASTRESGVPD |
| | | RFTGSGSVTDFTLTISSVQAEDLAVYYCQNDYS |
| | | YPCTFGQGTKVEIK |
| SEQ ID NO: 25 | DNA VL | GACATTGTGATGACCCAGTCTCCATCCTCCCTG |
| | | ACTGTGACAGCAGGAGAGAAGGTCACTATGAGC |
| | | TGCAAGTCCAGTCAGAGTCTGTTAGACAGTGGA |
| | | AATCAAAAGAACTTCTTGACCTGGTACCAGCAG |
| | | AAACCAGGGCAGCCTCCTAAACTGTTGATCTTC |
| | | TGGGCATCCACTAGGGAATCTGGGGTCCCTGAT |
| | | CGCTTCACAGGCAGTGGATCTGTAACAGATTTC |
| | | ACTCTCACCATCAGCAGTGTGCAGGCTGAAGAC |
| | | CTGGCAGTTTATTACTGTCAGAATGATTATAGT |
| | | TATCCGTGCACGTTCGGCCAAGGGACCAAGGTG |
| | | GAAATCAAA |
| SEQ ID NO: 26 | LC | DIVMTQSPSSLTVTAGEKVTMSCKSSQSLLDSG |
| | | NQKNFLTWYQQKPGQPPKLLIFWASTRESGVPD |
| | | RFTGSGSVTDFTLTISSVQAEDLAVYYCQNDYS |
| | | YPCTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 27 | DNA LC | GACATTGTGATGACCCAGTCTCCATCCTCCCTG |
| | | ACTGTGACAGCAGGAGAGAAGGTCACTATGAGC |
| | | TGCAAGTCCAGTCAGAGTCTGTTAGACAGTGGA |
| | | AATCAAAAGAACTTCTTGACCTGGTACCAGCAG |
| | | AAACCAGGGCAGCCTCCTAAACTGTTGATCTTC |
| | | TGGGCATCCACTAGGGAATCTGGGGTCCCTGAT |
| | | CGCTTCACAGGCAGTGGATCTGTAACAGATTTC |
| | | ACTCTCACCATCAGCAGTGTGCAGGCTGAAGAC |
| | | CTGGCAGTTTATTACTGTCAGAATGATTATAGT |
| | | TATCCGTGCACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-chi-Y HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 18 | VH | QVQLQQPGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSS |
| SEQ ID NO: 19 | DNA VH | CAGGTCCAGCTGCAGCAGCCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCC |
| SEQ ID NO: 20 | HC | QVQLQQPGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 21 | DNA HC | CAGGTCCAGCTGCAGCAGCCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| SEQ ID NO: 22 | VH | QVQLQQSGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSS |
| SEQ ID NO: 23 | DNA VH | CAGGTCCAGCTGCAGCAGTCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCC |
| SEQ ID NO: 30 | HC | QVQLQQSGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 31 | DNA HC | CAGGTCCAGCTGCAGCAGTCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-chi-Y LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 34 | VL | DIVMTQSPSSLTVTAGEKVTMSCKSSQSLLDSG |
| | | NQKNFLTWYQQKPGQPPKLLIFWASTRESGVPD |
| | | RFTGSGSVTDFTLTISSVQAEDLAVYYCQNDYS |
| | | YPYTFGQGTKVEIK |
| SEQ ID NO: 35 | DNA VL | GACATTGTGATGACCCAGTCTCCATCCTCCCTG |
| | | ACTGTGACAGCAGGAGAGAAGGTCACTATGAGC |
| | | TGCAAGTCCAGTCAGAGTCTGTTAGACAGTGGA |
| | | AATCAAAAGAACTTCTTGACCTGGTACCAGCAG |
| | | AAACCAGGGCAGCCTCCTAAACTGTTGATCTTC |
| | | TGGGCATCCACTAGGGAATCTGGGGTCCCTGAT |
| | | CGCTTCACAGGCAGTGGATCTGTAACAGATTTC |
| | | ACTCTCACCATCAGCAGTGTGCAGGCTGAAGAC |
| | | CTGGCAGTTTATTACTGTCAGAATGATTATAGT |
| | | TATCCGTACACGTTCGGCCAAGGGACCAAGGTG |
| | | LGAAATCAAA |
| SEQ ID NO: 36 | LC | DIVMTQSPSSLTVTAGEKVTMSCKSSQSLLDSG |
| | | NQKNFLTWYQQKPGQPPKLLIFWASTRESGVPD |
| | | RFTGSGSVTDFTLTISSVQAEDLAVYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 37 | DNA LC | GACATTGTGATGACCCAGTCTCCATCCTCCCTG |
| | | ACTGTGACAGCAGGAGAGAAGGTCACTATGAGC |
| | | TGCAAGTCCAGTCAGAGTCTGTTAGACAGTGGA |
| | | AATCAAAAGAACTTCTTGACCTGGTACCAGCAG |
| | | AAACCAGGGCAGCCTCCTAAACTGTTGATCTTC |
| | | TGGGCATCCACTAGGGAATCTGGGGTCCCTGAT |
| | | CGCTTCACAGGCAGTGGATCTGTAACAGATTTC |
| | | ACTCTCACCATCAGCAGTGTGCAGGCTGAAGAC |
| | | CTGGCAGTTTATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum01 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 39 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 40 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 41 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum01 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 42 | VL | EIVLTQSPATLSLSPGERATLSCKSSQSLLDSG |
| | | NQKNFLTWYQQKPGQAPRLLIYWASTRESGVPS |
| | | RFSGSGSGTEFTLTISSLQPDDFATYYCQNDYS |
| | | YPYTFGQGTKVEIK |
| SEQ ID NO: 43 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCATCA |
| | | AGGTTCAGCGGCAGTGGATCTGGGACAGAATTC |
| | | ACTCTCACCATCAGCAGCCTGCAGCCTGATGAT |
| | | TTTGCAACTTATTACTGTCAGAATGATTATAGT |
| | | TATCCGTACACGTTCGGCCAAGGGACCAAGGTG |
| | | GAAATCAAA |
| SEQ ID NO: 44 | LC | EIVLTQSPATLSLSPGERATLSCKSSQSLLDSG |
| | | NQKNFLTWYQQKPGQAPRLLIYWASTRESGVPS |
| | | RFSGSGSGTEFTLTISSLQPDDFATYYCQNDYS |
| | | YPYTEGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 45 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCATCA |
| | | AGGTTCAGCGGCAGTGGATCTGGGACAGAATTC |
| | | ACTCTCACCATCAGCAGCCTGCAGCCTGATGAT |
| | | TTTGCAACTTATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum02 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 6 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 39 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 40 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 41 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum02 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 46 | VL | DIQMTQSPSSLSASVGDRVTITCKSSQSLLDSG |
| | | NQKNFLTWYQQKPGQAPRLLIYWASTRESGIPP |
| | | RFSGSGYGTDFTLTINNIESEDAAYYFCQNDYS |
| | | YPYTFGQGTKVEIK |
| SEQ ID NO: 47 | DNA VL | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGATCCCACCT |
| | | CGATTCAGTGGCAGCGGGTATGGAACAGATTTT |
| | | ACCCTCACAATTAATAACATAGAATCTGAGGAT |
| | | GCTGCATATTACTTCTGTCAGAATGATTATAGT |
| | | TATCCGTACACGTTCGGCCAAGGGACCAAGGTG |
| | | GAAATCAAA |
| SEQ ID NO: 48 | LC | DIQMTQSPSSLSASVGDRVTITCKSSQSLLDSG |
| | | NQKNFLTWYQQKPGQAPRLLIYWASTRESGIPP |
| | | RFSGSGYGTDFTLTINNIESEDAAYYFCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 49 | DNA LC | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGATCCCACCT |
| | | CGATTCAGTGGCAGCGGGTATGGAACAGATTTT |
| | | ACCCTCACAATTAATAACATAGAATCTGAGGAT |
| | | GCTGCATATTACTTCTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum03 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 50 | VH | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 51 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 52 | HC | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 53 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum03 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 46 | VL | DIQMTQSPSSLSASVGDRVTITCKSSQSLLDSG |
| | | NQKNFLTWYQQKPGQAPRLLIYWASTRESGIPP |
| | | RFSGSGYGTDFTLTINNIESEDAAYYFCQNDYS |
| | | YPYTFGQGTKVEIK |
| SEQ ID NO: 47 | DNA VL | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGATCCCACCT |
| | | CGATTCAGTGGCAGCGGGTATGGAACAGATTTT |
| | | ACCCTCACAATTAATAACATAGAATCTGAGGAT |
| | | GCTGCATATTACTTCTGTCAGAATGATTATAGT |
| | | TATCCGTACACGTTCGGCCAAGGGACCAAGGTG |
| | | GAAATCAAA |
| SEQ ID NO: 48 | LC | DIQMTQSPSSLSASVGDRVTITCKSSQSLLDSG |
| | | NQKNFLTWYQQKPGQAPRLLIYWASTRESGIPP |
| | | RFSGSGYGTDFTLTINNIESEDAAYYFCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 49 | DNA LC | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGATCCCACCT |
| | | CGATTCAGTGGCAGCGGGTATGGAACAGATTTT |
| | | ACCCTCACAATTAATAACATAGAATCTGAGGAT |
| | | GCTGCATATTACTTCTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum04 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 50 | VH | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 51 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 52 | HC | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 53 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum04 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNE |
| SEQ ID NO: 14 (Chothia) | LCDR2 | WAS |
| SEQ ID NO: 33 (Chothia) | LCDR3 | DYSYPY |
| SEQ ID NO: 54 | VL | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIK |
| SEQ ID NO: 55 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCATCA |
| | | AGGTTCAGTGGAAGTGGATCTGGGACAGATTTT |
| | | ACTTTCACCATCAGCAGCCTGCAGCCTGAAGAT |
| | | ATTGCAACATATTACTGTCAGAATGATTATAGT |
| | | TATCCGTACACGTTCGGCCAAGGGACCAAGGTG |
| | | GAAATCAAA |
| SEQ ID NO: 56 | LC | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTGGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSENRGEC |
| SEQ ID NO: 57 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCATCA |
| | | AGGTTCAGTGGAAGTGGATCTGGGACAGATTTT |
| | | ACTTTCACCATCAGCAGCCTGCAGCCTGAAGAT |
| | | ATTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum05 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 39 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 40 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 41 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum05 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 54 | VL | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIK |
| SEQ ID NO: 55 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | GGGCATCCACTAGGGAATCTGGGGTCCCATCA |
| | | AGGTTCAGTGGAAGTGGATCTGGGACAGATTTT |
| | | ACTTTCACCATCAGCAGCCTGCAGCCTGAAGAT |
| | | ATTGCAACATATTACTGTCAGAATGATTATAGT |
| | | TATCCGTACACGTTCGGCCAAGGGACCAAGGTG |
| | | GAAATCAAA |
| SEQ ID NO: 56 | LC | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 57 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCATCA |
| | | AGGTTCAGTGGAAGTGGATCTGGGACAGATTTT |
| | | ACTTTCACCATCAGCAGCCTGCAGCCTGAAGAT |
| | | ATTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum06 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 39 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 40 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 41 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum06 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 58 | VL | DIVMTQTPLSLPVTPGEPASISCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 59 | DNA VL | GATATTGTGATGACCCAGACTCCACTCTCCCTG |
| | | CCCGTCACCCCTGGAGAGCCGGCCTCCATCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 60 | LC | DIVMTQTPLSLPVTPGEPASISCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 61 | DNA LC | GATATTGTGATGACCCAGACTCCACTCTCCCTG |
| | | CCCGTCACCCCTGGAGAGCCGGCCTCCATCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum07 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 39 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 40 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 41 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum07 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 62 | VL | EIVLTQSPATLSLSPGERATLSCKSSQSLLDSG |
| | | NQKNFLTWYQQKPGKAPKLLIYWASTRESGVPS |
| | | RFSGSGSGTDFTFTISSLEAEDAATYYCQNDYS |
| | | YPYTFGQGTKVEIK |
| SEQ ID NO: 63 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 64 | LC | EIVLTQSPATLSLSPGERATLSCKSSQSLLDSG |
| | | NQKNFLTWYQQKPGKAPKLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 65 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum08 HC | | |
| SEQ ID NO: 1 (Kabat) | HCDR1 | TPYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 50 | VH | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 51 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 52 | HC | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 53 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum08 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 66 | VL | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 67 | DNA VL | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 68 | LC | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 69 | DNA LC | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum09 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 39 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 40 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 41 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum09 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 66 | VL | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 67 | DNA VL | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 68 | LC | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 69 | DNA LC | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum10 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 50 | VH | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 51 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 52 | HC | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 53 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum10 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 70 | VL | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 71 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 72 | LC | EIVLTQSPATLSLSPGEPATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTEGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSENRGEC |
| SEQ ID NO: 73 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum11 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 39 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 40 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 41 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum11 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 70 | VL | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 71 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 72 | LC | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 73 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum12 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 39 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 40 | HC | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 41 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum12 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 74 | VL | DIQMTQSPSSLSASVGDRVTITCKSSQSLLDSG |
| | | NQKNFLTWYLQKPGQSPQLLIYWASTRESGVPS |
| | | RFSGSGSGTDFTFTISSLEAEDAATYYCQNDYS |
| | | YPYTFGQGTKVEIK |
| SEQ ID NO: 75 | DNA VL | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCTGCAG |
| | | AAGCCAGGGCAGTCTCCACAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 76 | LC | DIQMTQSPSSLSASVGDRVTITCKSSQSLLDSG |
| | | NQKNFLTWYLQKPGQSPQLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 77 | DNA LC | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCTGCAG |
| | | AAGCCAGGGCAGTCTCCACAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum13 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 39 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 40 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 41 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum13 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 78 | VL | DVVMTQSPLSLPVTLGQPASISCKSSQSLLDSG |
| | | NQKNFLTWYQQKPGKAPKLLIYWASTRESGVPS |
| | | RFSGSGSGTDFTFTISSLEAEDAATYYCQNDYS |
| | | YPYTFGQGTKVEIK |
| SEQ ID NO: 79 | DNA VL | GATGTTGTGATGACTCAGTCTCCACTCTCCCTG |
| | | CCCGTCACCCTTGGACAGCCGGCCTCCATCTCC |
| | | TGCAAGTCCAGTCAGAGTCTGTTAGACAGTGGA |
| | | AATCAAAAGAACTTCTTAACCTGGTATCAGCAG |
| | | AAACCAGGGAAAGCTCCTAAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 80 | LC | DVVMTQSPLSLPVTLGQPASISCKSSQSLLDSG |
| | | NQKNFLTWYQQKPGKAPKLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 81 | DNA LC | GATGTTGTGATGACTCAGTCTCCACTCTCCCTG |
| | | CCCGTCACCCTTGGACAGCCGGCCTCCATCTCC |
| | | TGCAAGTCCAGTCAGAGTCTGTTAGACAGTGGA |
| | | AATCAAAAGAACTTCTTAACCTGGTATCAGCAG |
| | | AAACCAGGGAAAGCTCCTAAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum14 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 82 | VH | QVQLVQSGAEVKKPGASVKVSCKASGYTFTTYW |
| | | MHWIRQSPSRGLEWLGNIYPGIGGSNFDEKFKN |
| | | RFTISRDNSKNTLYLQMNSLRAEDTAVYYCTRW |
| | | TTGTGAYWGQGTTVTVSS |
| SEQ ID NO: 83 | DNA VH | CAGGTTCAGCTGGTGCAGTCTGGAGCTGAGGTG |
| | | AAGAAGCCTGGGGCCTCAGTGAAGGTCTCCTGC |
| | | AAGGCTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTACTGGGGCCAGGGC |
| | | ACCACCGTGACCGTGTCCTCC |
| SEQ ID NO: 84 | HC | QVQLVQSGAEVKKPGASVKVSCKASGYTFTTYW |
| | | MHWIRQSPSRGLEWLGNIYPGTGGSNFDEKFKN |
| | | RFTISRDNSKNTLYLQMNSLRAEDTAVYYCTRW |
| | | TFGTGAYWGQGTFVFVSSASTKGPSVFPLAPCS |
| | | RSTSESTAALGCLVKDYFPEPVTVSWNSGALTS |
| | | GVHTFPAVLQSSGLYSLSSVVFVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 85 | DNA HC | CAGGTTCAGCTGGTGCAGTCTGGAGCTGAGGTG |
| | | AAGAAGCCTGGGGCCTCAGTGAAGGTCTCCTGC |
| | | AAGGCTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTACTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum14 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 70 | VL | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 71 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 72 | LC | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTEGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 73 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum15 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 82 | VH | QVQLVQSGAEVKKPGASVKVSCKASGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 83 | DNA VH | CAGGTTCAGCTGGTGCAGTCTGGAGCTGAGGTG |
| | | AAGAAGCCTGGGGCCTCAGTGAAGGTCTCCTGC |
| | | AAGGCTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTACTGGGGCCAGGGC |
| | | ACCACCGTGACCGTGTCCTCC |
| SEQ ID NO: 84 | HC | QVQLVQSGAEVKKPGASVKVSCKASGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 85 | DNA HC | CAGGTTCAGCTGGTGCAGTCTGGAGCTGAGGTG |
| | | AAGAAGCCTGGGGCCTCAGTGAAGGTCTCCTGC |
| | | AAGGCTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTACTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum15 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 66 | VL | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 67 | DNA VL | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 68 | LC | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 69 | DNA LC | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-hum16 HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 86 | VH | EVQLVQSGAEVKKPGESLRISCKGSGYTFTTYW |
| | | MHWVRQAPGQGLEWMGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 87 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCCCTGGACAAGGG |
| | | CTTGAGTGGATGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 88 | HC | EVQLVQSGAEVKKPGESLRISCKGSGYTFTTYW |
| | | MHWVRQAPGQGLEWMGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 89 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCCCTGGACAAGGG |
| | | CTTGAGTGGATGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| BAP049-hum16 LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 66 | VL | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 67 | DNA VL | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 68 | LC | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSENRGEC |
| SEQ ID NO: 69 | DNA LC | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |
| BAP049-Clone-A HC | | |
| SEQ ID NO: 1 (Kabat) | HCDR1 | TYWMH |
| SEQ ID NO: 2 (Kabat) | HCDR2 | NIYPGIGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 90 | DNA VH | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGGTGCGACAGGCTACCGGCCAGGGC |
| | | CTGGAATGGATGGGCAACATCTATCCTGGCACC |
| | | GGCGGCTCCAACTTCGACGAGAAGTTCAAGAAC |
| | | AGAGTGACCATCACCGCCGACAAGTCCACCTCC |
| | | ACCGCCTACATGGAACTGTCCTCCCTGAGATCC |
| | | GAGGACACCGCCGTGTACTACTGCACCCGGTGG |
| | | ACAACCGGCACAGGCGCTTATTGGGGCCAGGGC |
| | | ACCACAGTGACCGTGTCCTCT |
| SEQ ID NO: 91 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLG |
| SEQ ID NO: 92 | DNA HC | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGGTGCGACAGGCTACCGGCCAGGGC |
| | | CTGGAATGGATGGGCAACATCTATCCTGGCACC |
| | | GGCGGCTCCAACTTCGACGAGAAGTTCAAGAAC |
| | | AGAGTGACCATCACCGCCGACAAGTCCACCTCC |
| | | ACCGCCTACATGGAACTGTCCTCCCTGAGATCC |
| | | GAGGACACCGCCGTGTACTACTGCACCCGGTGG |
| | | ACAACCGGCACAGGCGCTTATTGGGGCCAGGGC |
| | | ACCACAGTGACCGTGTCCTCTGCTTCTACCAAG |
| | | GGGCCCAGCGTGTTCCCCCTGGCCCCCTGCTCC |
| | | AGAAGCACCAGCGAGAGCACAGCCGCCCTGGGC |
| | | TGCCTGGTGAAGGACTACTTCCCCGAGCCCGTG |
| | | ACCGTGTCCTGGAACAGCGGAGCCCTGACCAGC |
| | | GGCGTGCACACCTTCCCCGCCGTGCTGCAGAGC |
| | | AGCGGCCTGTACAGCCTGAGCAGCGTGGTGACC |
| | | GTGCCCAGCAGCAGCCTGGGCACCAAGACCTAC |
| | | ACCTGTAACGTGGACCACAAGCCCAGCAACACC |
| | | AAGGTGGACAAGAGGGTGGAGAGCAAGTACGGC |
| | | CCACCCTGCCCCCCCTGCCCAGCCCCCGAGTTC |
| | | CTGGGCGGACCCAGCGTGTTCCTGTTCCCCCCC |
| | | AAGCCCAAGGACACCCTGATGATCAGCAGAACC |
| | | CCCGAGGTGACCTGTGTGGTGGTGGACGTGTCC |
| | | CAGGAGGACCCCGAGGTCCAGTTCAACTGGTAC |
| | | GTGGACGGCGTGGAGGTGCACAACGCCAAGACC |
| | | AAGCCCAGAGAGGAGCAGTTTAACAGCACCTAC |
| | | CGGGTGGTGTCCGTGCTGACCGTGCTGCACCAG |
| | | GACTGGCTGAACGGCAAAGAGTACAAGTGTAAG |
| | | GTCTCCAACAAGGGCCTGCCAAGCAGCATCGAA |
| | | AAGACCATCAGCAAGGCCAAGGGCCAGCCTAGA |
| | | GAGCCCCAGGTCTACACCCTGCCACCCAGCCAA |
| | | GAGGAGATGACCAAGAACCAGGTGTCCCTGACC |
| | | TGTCTGGTGAAGGGCTTCTACCCAAGCGACATC |
| | | GCCGTGGAGTGGGAGAGCAACGGCCAGCCCGAG |
| | | AACAACTACAAGACCACCCCCCCAGTGCTGGAC |
| | | AGCGACGGCAGCTTCTTCCTGTACAGCAGGCTG |
| | | ACCGTGGACAAGTCCAGATGGCAGGAGGGCAAC |
| | | GTCTTTAGCTGCTCCGTGATGCACGAGGCCCTG |
| | | CACAACCACTACACCCAGAAGAGCCTGAGCCTG |
| | | TCCCTGGGC |
| BAP049-Clone-A LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 42 | VL | EIVLTQSPATLSLSPGERATLSCKSSQSLLDSG |
| | | NQKNFLTWYQQKPGQAPRLLIYWASTRESGVPS |
| | | RFSGSGSGTEFTLTISSLQPDDFATYYCQNDYS |
| | | YPYTFGQGTKVEIK |
| SEQ ID NO: 93 | DNA VL | GAGATCGTGCTGACCCAGTCCCCTGCCACCCTG |
| | | TCACTGTCTCCAGGCGAGAGAGCTACCCTGTCC |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGAGTTT |
| | | ACCCTGACCATCTCCAGCCTGCAGCCCGACGAC |
| | | TTCGCCACCTACTACTGCCAGAACGACTACTCC |
| | | TACCCCTACACCTTCGGCCAGGGCACCAAGGTG |
| | | GAAATCAAG |
| SEQ ID NO: 44 | LC | EIVLTQSPATLSLSPGERATLSCKSSQSLLDSG |
| | | NQKNFLTWYQQKPGQAPRLLIYWASTRESGVPS |
| | | RFSGSGSGTEFTLTISSLQPDDFATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 94 | DNA LC | GAGATCGTGCTGACCCAGTCCCCTGCCACCCTG |
| | | TCACTGTCTCCAGGCGAGAGAGCTACCCTGTCC |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGAGTTT |
| | | ACCCTGACCATCTCCAGCCTGCAGCCCGACGAC |
| | | TTCGCCACCTACTACTGCCAGAACGACTACTCC |
| | | TACCCCTACACCTTCGGCCAGGGCACCAAGGTG |
| | | GAAATCAAGCGTACGGTGGCCGCTCCCAGCGTG |
| | | TTCATCTTCCCCCCAAGCGACGAGCAGCTGAAG |
| | | AGCGGCACCGCCAGCGTGGTGTGTCTGCTGAAC |
| | | AACTTCTACCCCAGGGAGGCCAAGGTGCAGTGG |
| | | AAGGTGGACAACGCCCTGCAGAGCGGCAACAGC |
| | | CAGGAGAGCGTCACCGAGCAGGACAGCAAGGAC |
| | | TCCACCTACAGCCTGAGCAGCACCCTGACCCTG |
| | | AGCAAGGCCGACTACGAGAAGCACAAGGTGTAC |
| | | GCCTGTGAGGTGACCCACCAGGGCCTGTCCAGC |
| | | CCCGTGACCAAGAGCTTCAACAGGGGCGAGTGC |
| BAP049-Clone-B HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 95 | DNA VH | GAGGTGCAGCTGGTGCAGTCAGGCGCCGAAGTG |
| | | AAGAAGCCCGGCGAGTCACTGAGAATTAGCTGT |
| | | AAAGGTTCAGGCTACACCTTCACTACCTACTGG |
| | | ATGCACTGGGTCCGCCAGGCTACCGGTCAAGGC |
| | | CTCGAGTGGATGGGTAATATCTACCCCGGCACC |
| | | GGCGGCTCTAACTTCGACGAGAAGTTTAAGAAT |
| | | AGAGTGACTATCACCGCCGATAAGTCTACTAGC |
| | | ACCGCCTATATGGAACTGTCTAGCCTGAGATCA |
| | | GAGGACACCGCCGTCTACTACTGCACTAGGTGG |
| | | ACTACCGGCACAGGCGCCTACTGGGGTCAAGGC |
| | | ACTACCGTGACCGTGTCTAGC |
| SEQ ID NO: 91 | HC | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLG |
| SEQ ID NO: 96 | DNA HC | GAGGTGCAGCTGGTGCAGTCAGGCGCCGAAGTG |
| | | AAGAAGCCCGGCGAGTCACTGAGAATTAGCTGT |
| | | AAAGGTTCAGGCTACACCTTCACTACCTACTGG |
| | | ATGCACTGGGTCCGCCAGGCTACCGGTCAAGGC |
| | | CTCGAGTGGATGGGTAATATCTACCCCGGCACC |
| | | GGCGGCTCTAACTTCGACGAGAAGTTTAAGAAT |
| | | AGAGTGACTATCACCGCCGATAAGTCTACTAGC |
| | | ACCGCCTATATGGAACTGTCTAGCCTGAGATCA |
| | | GAGGACACCGCCGTCTACTACTGCACTAGGTGG |
| | | ACTACCGGCACAGGCGCCTACTGGGGTCAAGGC |
| | | ACTACCGTGACCGTGTCTAGCGCTAGCACTAAG |
| | | GGCCCGTCCGTGTTCCCCCTGGCACCTTGTAGC |
| | | CGGAGCACTAGCGAATCCACCGCTGCCCTCGGC |
| | | TGCCTGGTCAAGGATTACTTCCCGGAGCCCGTG |
| | | ACCGTGTCCTGGAACAGCGGAGCCCTGACCTCC |
| | | GGAGTGCACACCTTCCCCGCTGTGCTGCAGAGC |
| | | TCCGGGCTGTACTCGCTGTCGTCGGTGGTCACG |
| | | GTGCCTTCATCTAGCCTGGGTACCAAGACCTAC |
| | | ACTTGCAACGTGGACCACAAGCCTTCCAACACT |
| | | AAGGTGGACAAGCGCGTCGAATCGAAGTACGGC |
| | | CCACCGTGCCCGCCTTGTCCCGCGCCGGAGTTC |
| | | CTCGGCGGTCCCTCGGTCTTTCTGTTCCCACCG |
| | | AAGCCCAAGGACACTTTGATGATTTCCCGCACC |
| | | CCTGAAGTGACATGCGTGGTCGTGGACGTGTCA |
| | | CAGGAAGATCCGGAGGTGCAGTTCAATTGGTAC |
| | | GTGGATGGCGTCGAGGTGCACAACGCCAAAACC |
| | | AAGCCGAGGGAGGAGCAGTTCAACTCCACTTAC |
| | | CGCGTCGTGTCCGTGCTGACGGTGCTGCATCAG |
| | | GACTGGCTGAACGGGAAGGAGTACAAGTGCAAA |
| | | GTGTCCAACAAGGGACTTCCTAGCTCAATCGAA |
| | | AAGACCATCTCGAAAGCCAAGGGACAGCCCCGG |
| | | GAACCCCAAGTGTATACCCTGCCACCGAGCCAG |
| | | GAAGAAATGACTAAGAACCAAGTCTCATTGACT |
| | | TGCCTTGTGAAGGGCTTCTACCCATCGGATATC |
| | | GCCGTGGAATGGGAGTCCAACGGCCAGCCGGAA |
| | | AACAACTACAAGACCACCCCTCCGGTGCTGGAC |
| | | TCAGACGGATCCTTCTTCCTCTACTCGCGGCTG |
| | | ACCGTGGATAAGAGCAGATGGCAGGAGGGAAAT |
| | | GTGTTCAGCTGTTCTGTGATGCATGAAGCCCTG |
| | | CACAACCACTACACTCAGAAGTCCCTGTCCCTC |
| | | TCCCTGGGA |
| BAP049-Clone-B LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 54 | VL | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIK |
| SEQ ID NO: 97 | DNA VL | GAGATCGTCCTGACTCAGTCACCCGCTACCCTG |
| | | AGCCTGAGCCCTGGCGAGCGGGCTACACTGAGC |
| | | TGTAAATCTAGTCAGTCACTGCTGGATAGCGGT |
| | | AATCAGAAGAACTTCCTGACCTGGTATCAGCAG |
| | | AAGCCCGGTAAAGCCCCTAAGCTGCTGATCTAC |
| | | TGGGCCTCTACTAGAGAATCAGGCGTGCCCTCT |
| | | AGGTTTAGCGGTAGCGGTAGTGGCACCGACTTC |
| | | ACCTTCACTATCTCTAGCCTGCAGCCCGAGGAT |
| | | ATCGCTACCTACTACTGTCAGAACGACTATAGC |
| | | TACCCCTACACCTTCGGTCAAGGCACTAAGGTC |
| | | GAGATTAAG |
| SEQ ID NO: 56 | LC | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 98 | DNA LC | GAGATCGTCCTGACTCAGTCACCCGCTACCCTG |
| | | AGCCTGAGCCCTGGCGAGCGGGCTACACTGAGC |
| | | TGTAAATCTAGTCAGTCACTGCTGGATAGCGGT |
| | | AATCAGAAGAACTTCCTGACCTGGTATCAGCAG |
| | | AAGCCCGGTAAAGCCCCTAAGCTGCTGATCTAC |
| | | TGGGCCTCTACTAGAGAATCAGGCGTGCCCTCT |
| | | AGGTTTAGCGGTAGCGGTAGTGGCACCGACTTC |
| | | ACCTTCACTATCTCTAGCCTGCAGCCCGAGGAT |
| | | ATCGCTACCTACTACTGTCAGAACGACTATAGC |
| | | TACCCCTACACCTTCGGTCAAGGCACTAAGGTC |
| | | GAGATTAAGCGTACGGTGGCCGCTCCCAGCGTG |
| | | TTCATCTTCCCCCCCAGCGACGAGCAGCTGAAG |
| | | AGCGGCACCGCCAGCGTGGTGTGCCTGCTGAAC |
| | | AACTTCTACCCCCGGGAGGCCAAGGTGCAGTGG |
| | | AAGGTGGACAACGCCCTGCAGAGCGGCAACAGC |
| | | CAGGAGAGCGTCACCGAGCAGGACAGCAAGGAC |
| | | TCCACCTACAGCCTGAGCAGCACCCTGACCCTG |
| | | AGCAAGGCCGACTACGAGAAGCATAAGGTGTAC |
| | | GCCTGCGAGGTGACCCACCAGGGCCTGTCCAGC |
| | | CCCGTGACCAAGAGCTTCAACAGGGGCGAGTGC |
| BAP049-Clone-C HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 90 | DNA VH | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGGTGCGACAGGCTACCGGCCAGGGC |
| | | CTGGAATGGATGGGCAACATCTATCCTGGCACC |
| | | GGCGGCTCCAACTTCGACGAGAAGTTCAAGAAC |
| | | AGAGTGACCATCACCGCCGACAAGTCCACCTCC |
| | | ACCGCCTACATGGAACTGTCCTCCCTGAGATCC |
| | | GAGGACACCGCCGTGTACTACTGCACCCGGTGG |
| | | ACAACCGGCACAGGCGCTTATTGGGGCCAGGGC |
| | | ACCACAGTGACCGTGTCCTCT |
| SEQ ID NO: 91 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLG |
| SEQ ID NO: 92 | DNA HC | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGGTGCGACAGGCTACCGGCCAGGGC |
| | | CTGGAATGGATGGGCAACATCTATCCTGGCACC |
| | | GGCGGCTCCAACTTCGACGAGAAGTTCAAGAAC |
| | | AGAGTGACCATCACCGCCGACAAGTCCACCTCC |
| | | ACCGCCTACATGGAACTGTCCTCCCTGAGATCC |
| | | GAGGACACCGCCGTGTACTACTGCACCCGGTGG |
| | | ACAACCGGCACAGGCGCTTATTGGGGCCAGGGC |
| | | ACCACAGTGACCGTGTCCTCTGCTTCTACCAAG |
| | | GGGCCCAGCGTGTTCCCCCTGGCCCCCTGCTCC |
| | | AGAAGCACCAGCGAGAGCACAGCCGCCCTGGGC |
| | | TGCCTGGTGAAGGACTACTTCCCCGAGCCCGTG |
| | | ACCGTGTCCTGGAACAGCGGAGCCCTGACCAGC |
| | | GGCGTGCACACCTTCCCCGCCGTGCTGCAGAGC |
| | | AGCGGCCTGTACAGCCTGAGCAGCGTGGTGACC |
| | | GTGCCCAGCAGCAGCCTGGGCACCAAGACCTAC |
| | | ACCTGTAACGTGGACCACAAGCCCAGCAACACC |
| | | AAGGTGGACAAGAGGGTGGAGAGCAAGTACGGC |
| | | CCACCCTGCCCCCCCTGCCCAGCCCCCGAGTTC |
| | | CTGGGCGGACCCAGCGTGTTCCTGTTCCCCCCC |
| | | AAGCCCAAGGACACCCTGATGATCAGCAGAACC |
| | | CCCGAGGTGACCTGTGTGGTGGTGGACGTGTCC |
| | | CAGGAGGACCCCGAGGTCCAGTTCAACTGGTAC |
| | | GTGGACGGCGTGGAGGTGCACAACGCCAAGACC |
| | | AAGCCCAGAGAGGAGCAGTTTAACAGCACCTAC |
| | | CGGGTGGTGTCCGTGCTGACCGTGCTGCACCAG |
| | | GACTGGCTGAACGGCAAAGAGTACAAGTGTAAG |
| | | GTCTCCAACAAGGGCCTGCCAAGCAGCATCGAA |
| | | AAGACCATCAGCAAGGCCAAGGGCCAGCCTAGA |
| | | GAGCCCCAGGTCTACACCCTGCCACCCAGCCAA |
| | | GAGGAGATGACCAAGAACCAGGTGTCCCTGACC |
| | | TGTCTGGTGAAGGGCTTCTACCCAAGCGACATC |
| | | GCCGTGGAGTGGGAGAGCAACGGCCAGCCCGAG |
| | | AACAACTACAAGACCACCCCCCCAGTGCTGGAC |
| | | AGCGACGGCAGCTTCTTCCTGTACAGCAGGCTG |
| | | ACCGTGGACAAGTCCAGATGGCAGGAGGGCAAC |
| | | GTCTTTAGCTGCTCCGTGATGCACGAGGCCCTG |
| | | CACAACCACTACACCCAGAAGAGCCTGAGCCTG |
| | | TCCCTGGGC |
| BAP04 9-Clone-C LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 66 | VL | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 99 | DNA VL | GAGATCGTGCTGACCCAGTCCCCCGACTTCCAG |
| | | TCCGTGACCCCCAAAGAAAAAGTGACCATCACA |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGACTTT |
| | | ACCTTCACCATCTCCAGCCTGGAAGCCGAGGAC |
| | | GCCGCCACCTACTACTGCCAGAACGACTACTCC |
| | | TACCCCTACACCTTCGGCCAGGGCACCAAGGTG |
| | | GAAATCAAG |
| SEQ ID NO: 68 | LC | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 100 | DNA LC | GAGATCGTGCTGACCCAGTCCCCCGACTTCCAG |
| | | TCCGTGACCCCCAAAGAAAAAGTGACCATCACA |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGACTTT |
| | | ACCTTCACCATCTCCAGCCTGGAAGCCGAGGAC |
| | | GCCGCCACCTACTACTGCCAGAACGACTACTCC |
| | | TACCCCTACACCTTCGGCCAGGGCACCAAGGTG |
| | | GAAATCAAGCGTACGGTGGCCGCTCCCAGCGTG |
| | | TTCATCTTCCCCCCAAGCGACGAGCAGCTGAAG |
| | | AGCGGCACCGCCAGCGTGGTGTGTCTGCTGAAC |
| | | AACTTCTACCCCAGGGAGGCCAAGGTGCAGTGG |
| | | AAGGTGGACAACGCCCTGCAGAGCGGCAACAGC |
| | | CAGGAGAGCGTCACCGAGCAGGACAGCAAGGAC |
| | | TCCACCTACAGCCTGAGCAGCACCCTGACCCTG |
| | | AGCAAGGCCGACTACGAGAAGCACAAGGTGTAC |
| | | GCCTGTGAGGTGACCCACCAGGGCCTGTCCAGC |
| | | CCCGTGACCAAGAGCTTCAACAGGGGCGAGTGC |
| BAP049-Clone-D HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 50 | VH | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | FTGTGAYWGQGTTVTVSS |
| SEQ ID NO: 101 | DNA VH | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGATCCGGCAGTCCCCCTCTAGGGGC |
| | | CTGGAATGGCTGGGCAACATCTACCCTGGCACC |
| | | GGCGGCTCCAACTTCGACGAGAAGTTCAAGAAC |
| | | AGGTTCACCATCTCCCGGGACAACTCCAAGAAC |
| | | ACCCTGTACCTGCAGATGAACTCCCTGCGGGCC |
| | | GAGGACACCGCCGTGTACTACTGTACCAGATGG |
| | | ACCACCGGAACCGGCGCCTATTGGGGCCAGGGC |
| | | ACAACAGTGACCGTGTCCTCC |
| SEQ ID NO: 102 | HC | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLG |
| SEQ ID NO: 103 | DNA HC | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGATCCGGCAGTCCCCCTCTAGGGGC |
| | | CTGGAATGGCTGGGCAACATCTACCCTGGCACC |
| | | GGCGGCTCCAACTTCGACGAGAAGTTCAAGAAC |
| | | AGGTTCACCATCTCCCGGGACAACTCCAAGAAC |
| | | ACCCTGTACCTGCAGATGAACTCCCTGCGGGCC |
| | | GAGGACACCGCCGTGTACTACTGTACCAGATGG |
| | | ACCACCGGAACCGGCGCCTATTGGGGCCAGGGC |
| | | ACAACAGTGACCGTGTCCTCCGCTTCTACCAAG |
| | | GGGCCCAGCGTGTTCCCCCTGGCCCCCTGCTCC |
| | | AGAAGCACCAGCGAGAGCACAGCCGCCCTGGGC |
| | | TGCCTGGTGAAGGACTACTTCCCCGAGCCCGTG |
| | | ACCGTGTCCTGGAACAGCGGAGCCCTGACCAGC |
| | | GGCGTGCACACCTTCCCCGCCGTGCTGCAGAGC |
| | | AGCGGCCTGTACAGCCTGAGCAGCGTGGTGACC |
| | | GTGCCCAGCAGCAGCCTGGGCACCAAGACCTAC |
| | | ACCTGTAACGTGGACCACAAGCCCAGCAACACC |
| | | AAGGTGGACAAGAGGGTGGAGAGCAAGTACGGC |
| | | CCACCCTGCCCCCCCTGCCCAGCCCCCGAGTTC |
| | | CTGGGCGGACCCAGCGTGTTCCTGTTCCCCCCC |
| | | AAGCCCAAGGACACCCTGATGATCAGCAGAACC |
| | | CCCGAGGTGACCTGTGTGGTGGTGGACGTGTCC |
| | | CAGGAGGACCCCGAGGTCCAGTTCAACTGGTAC |
| | | GTGGACGGCGTGGAGGTGCACAACGCCAAGACC |
| | | AAGCCCAGAGAGGAGCAGTTTAACAGCACCTAC |
| | | CGGGTGGTGTCCGTGCTGACCGTGCTGCACCAG |
| | | GACTGGCTGAACGGCAAAGAGTACAAGTGTAAG |
| | | GTCTCCAACAAGGGCCTGCCAAGCAGCATCGAA |
| | | AAGACCATCAGCAAGGCCAAGGGCCAGCCTAGA |
| | | GAGCCCCAGGTCTACACCCTGCCACCCAGCCAA |
| | | GAGGAGATGACCAAGAACCAGGTGTCCCTGACC |
| | | TGTCTGGTGAAGGGCTTCTACCCAAGCGACATC |
| | | GCCGTGGAGTGGGAGAGCAACGGCCAGCCCGAG |
| | | AACAACTACAAGACCACCCCCCCAGTGCTGGAC |
| | | AGCGACGGCAGCTTCTTCCTGTACAGCAGGCTG |
| | | ACCGTGGACAAGTCCAGATGGCAGGAGGGCAAC |
| | | GTCTTTAGCTGCTCCGTGATGCACGAGGCCCTG |
| | | CACAACCACTACACCCAGAAGAGCCTGAGCCTG |
| | | TCCCTGGGC |
| BAP04 9-Clone-D LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | LQNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 70 | VL | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 104 | DNA VL | GAGATCGTGCTGACCCAGTCCCCTGCCACCCTG |
| | | TCACTGTCTCCAGGCGAGAGAGCTACCCTGTCC |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGACTTT |
| | | ACCTTCACCATCTCCAGCCTGGAAGCCGAGGAC |
| | | GCCGCCACCTACTACTGCCAGAACGACTACTCC |
| | | TACCCCTACACCTTCGGCCAGGGCACCAAGGTG |
| | | GAAATCAAG |
| SEQ ID NO: 72 | LC | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTEGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 105 | DNA LC | GAGATCGTGCTGACCCAGTCCCCTGCCACCCTG |
| | | TCACTGTCTCCAGGCGAGAGAGCTACCCTGTCC |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGACTTT |
| | | ACCTTCACCATCTCCAGCCTGGAAGCCGAGGAC |
| | | GCCGCCACCTACTACTGCCAGAACGACTACTCC |
| | | TACCCCTACACCTTCGGCCAGGGCACCAAGGTG |
| | | GAAATCAAGCGTACGGTGGCCGCTCCCAGCGTG |
| | | TTCATCTTCCCCCCAAGCGACGAGCAGCTGAAG |
| | | AGCGGCACCGCCAGCGTGGTGTGTCTGCTGAAC |
| | | AACTTCTACCCCAGGGAGGCCAAGGTGCAGTGG |
| | | AAGGTGGACAACGCCCTGCAGAGCGGCAACAGC |
| | | CAGGAGAGCGTCACCGAGCAGGACAGCAAGGAC |
| | | TCCACCTACAGCCTGAGCAGCACCCTGACCCTG |
| | | AGCAAGGCCGACTACGAGAAGCACAAGGTGTAC |
| | | GCCTGTGAGGTGACCCACCAGGGCCTGTCCAGC |
| | | CCCGTGACCAAGAGCTTCAACAGGGGCGAGTGC |
| BAP049-Clone-E HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 95 | DNA VH | GAGGTGCAGCTGGTGCAGTCAGGCGCCGAAGTG |
| | | AAGAAGCCCGGCGAGTCACTGAGAATTAGCTGT |
| | | AAAGGTTCAGGCTACACCTTCACTACCTACTGG |
| | | ATGCACTGGGTCCGCCAGGCTACCGGTCAAGGC |
| | | CTCGAGTGGATGGGTAATATCTACCCCGGCACC |
| | | GGCGGCTCTAACTTCGACGAGAAGTTTAAGAAT |
| | | AGAGTGACTATCACCGCCGATAAGTCTACTAGC |
| | | ACCGCCTATATGGAACTGTCTAGCCTGAGATCA |
| | | GAGGACACCGCCGTCTACTACTGCACTAGGTGG |
| | | ACTACCGGCACAGGCGCCTACTGGGGTCAAGGC |
| | | ACTACCGTGACCGTGTCTAGC |
| SEQ ID NO: 91 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLIVDKSRWQEGNVFSCSVMHEAL |
| | | HNHYTQKSLSLSLG |
| SEQ ID NO: 96 | DNA HC | GAGGTGCAGCTGGTGCAGTCAGGCGCCGAAGTG |
| | | AAGAAGCCCGGCGAGTCACTGAGAATTAGCTGT |
| | | AAAGGTTCAGGCTACACCTTCACTACCTACTGG |
| | | ATGCACTGGGTCCGCCAGGCTACCGGTCAAGGC |
| | | CTCGAGTGGATGGGTAATATCTACCCCGGCACC |
| | | GGCGGCTCTAACTTCGACGAGAAGTTTAAGAAT |
| | | AGAGTGACTATCACCGCCGATAAGTCTACTAGC |
| | | ACCGCCTATATGGAACTGTCTAGCCTGAGATCA |
| | | GAGGACACCGCCGTCTACTACTGCACTAGGTGG |
| | | ACTACCGGCACAGGCGCCTACTGGGGTCAAGGC |
| | | ACTACCGTGACCGTGTCTAGCGCTAGCACTAAG |
| | | GGCCCGTCCGTGTTCCCCCTGGCACCTTGTAGC |
| | | CGGAGCACTAGCGAATCCACCGCTGCCCTCGGC |
| | | TGCCTGGTCAAGGATTACTTCCCGGAGCCCGTG |
| | | ACCGTGTCCTGGAACAGCGGAGCCCTGACCTCC |
| | | GGAGTGCACACCTTCCCCGCTGTGCTGCAGAGC |
| | | TCCGGGCTGTACTCGCTGTCGTCGGTGGTCACG |
| | | GTGCCTTCATCTAGCCTGGGTACCAAGACCTAC |
| | | ACTTGCAACGTGGACCACAAGCCTTCCAACACT |
| | | AAGGTGGACAAGCGCGTCGAATCGAAGTACGGC |
| | | CCACCGTGCCCGCCTTGTCCCGCGCCGGAGTTC |
| | | CTCGGCGGTCCCTCGGTCTTTCTGTTCCCACCG |
| | | AAGCCCAAGGACACTTTGATGATTTCCCGCACC |
| | | CCTGAAGTGACATGCGTGGTCGTGGACGTGTCA |
| | | CAGGAAGATCCGGAGGTGCAGTTCAATTGGTAC |
| | | GTGGATGGCGTCGAGGTGCACAACGCCAAAACC |
| | | AAGCCGAGGGAGGAGCAGTTCAACTCCACTTAC |
| | | CGCGTCGTGTCCGTGCTGACGGTGCTGCATCAG |
| | | GACTGGCTGAACGGGAAGGAGTACAAGTGCAAA |
| | | GTGTCCAACAAGGGACTTCCTAGCTCAATCGAA |
| | | AAGACCATCTCGAAAGCCAAGGGACAGCCCCGG |
| | | GAACCCCAAGTGTATACCCTGCCACCGAGCCAG |
| | | GAAGAAATGACTAAGAACCAAGTCTCATTGACT |
| | | TGCCTTGTGAAGGGCTTCTACCCATCGGATATC |
| | | GCCGTGGAATGGGAGTCCAACGGCCAGCCGGAA |
| | | AACAACTACAAGACCACCCCTCCGGTGCTGGAC |
| | | TCAGACGGATCCTTCTTCCTCTACTCGCGGCTG |
| | | ACCGTGGATAAGAGCAGATGGCAGGAGGGAAAT |
| | | GTGTTCAGCTGTTCTGTGATGCATGAAGCCCTG |
| | | CACAACCACTACACTCAGAAGTCCCTGTCCCTC |
| | | TCCCTGGGA |
| BAP049-Clone-E LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 70 | VL | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 106 | DNA VL | GAGATCGTCCTGACTCAGTCACCCGCTACCCTG |
| | | AGCCTGAGCCCTGGCGAGCGGGCTACACTGAGC |
| | | TGTAAATCTAGTCAGTCACTGCTGGATAGCGGT |
| | | AATCAGAAGAACTTCCTGACCTGGTATCAGCAG |
| | | AAGCCCGGTCAAGCCCCTAGACTGCTGATCTAC |
| | | TGGGCCTCTACTAGAGAATCAGGCGTGCCCTCT |
| | | AGGTTTAGCGGTAGCGGTAGTGGCACCGACTTC |
| | | ACCTTCACTATCTCTAGCCTGGAAGCCGAGGAC |
| | | GCCGCTACCTACTACTGTCAGAACGACTATAGC |
| | | TACCCCTACACCTTCGGTCAAGGCACTAAGGTC |
| | | GAGATTAAG |
| SEQ ID NO: 72 | LC | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIEPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 107 | DNA LC | GAGATCGTCCTGACTCAGTCACCCGCTACCCTG |
| | | AGCCTGAGCCCTGGCGAGCGGGCTACACTGAGC |
| | | TGTAAATCTAGTCAGTCACTGCTGGATAGCGGT |
| | | AATCAGAAGAACTTCCTGACCTGGTATCAGCAG |
| | | AAGCCCGGTCAAGCCCCTAGACTGCTGATCTAC |
| | | TGGGCCTCTACTAGAGAATCAGGCGTGCCCTCT |
| | | AGGTTTAGCGGTAGCGGTAGTGGCACCGACTTC |
| | | ACCTTCACTATCTCTAGCCTGGAAGCCGAGGAC |
| | | GCCGCTACCTACTACTGTCAGAACGACTATAGC |
| | | TACCCCTACACCTTCGGTCAAGGCACTAAGGTC |
| | | GAGATTAAGCGTACGGTGGCCGCTCCCAGCGTG |
| | | TTCATCTTCCCCCCCAGCGACGAGCAGCTGAAG |
| | | AGCGGCACCGCCAGCGTGGTGTGCCTGCTGAAC |
| | | AACTTCTACCCCCGGGAGGCCAAGGTGCAGTGG |
| | | AAGGTGGACAACGCCCTGCAGAGCGGCAACAGC |
| | | CAGGAGAGCGTCACCGAGCAGGACAGCAAGGAC |
| | | TCCACCTACAGCCTGAGCAGCACCCTGACCCTG |
| | | AGCAAGGCCGACTACGAGAAGCATAAGGTGTAC |
| | | GCCTGCGAGGTGACCCACCAGGGCCTGTCCAGC |
| | | CCCGTGACCAAGAGCTTCAACAGGGGCGAGTGC |
| BAP049 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | PPGGGCATCCACTAGGGAATCT |
| SEQ ID NO: 115 (Kabat) | LCDR3 | CAGAATGATTATAGTTATCCGTGCACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 118 (Chothia) | LCDR3 | GATTATAGTTATCCGTGC |
| BAP049-chi HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-chi LC | | |
| SEQ ID NO: 113 (Kabat) | LCDR1 | CAAAAGAACTTCTTGACC |
| SEQ ID NO: 114 (Kabat) | LCDR2 | TGGGCATCCACTAGGGAATCT |
| SEQ ID NO: 115 (Kabat) | LCDR3 | CAGAATGATTATAGTTATCCGTGCACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 118 (Chothia) | LCDR3 | GATTATAGTTATCCGTGC |
| BAP049-chi Y HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-chi Y LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum01 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum01 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum02 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum02 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum03 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum03 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum04 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum04 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum05 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum05 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum06 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum06 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum07 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum07 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum08 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum08 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum09 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum09 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID_NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum10 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum10 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum11 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum11 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum12 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum12 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum13 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum13 LC | | |
| SEQ ID NO: 121 (Kabat) | LCDR1 | AAGTCCAGTCAGAGTCTGTTAGACAGTGGAAAT |
| | | CAAAAGAACTTCTTAACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum14 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 223 (Kabat) | HCDR3 | TGGACTACTGGGACGGGAGCTTAC |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 223 (Chothia) | HCDR3 | TGGACTACTGGGACGGGAGCTTAC |
| BAP049-hum14 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum15 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 223 (Kabat) | HCDR3 | TGGACTACTGGGACGGGAGCTTAC |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 223 (Chothia) | HCDR3 | TGGACTACTGGGACGGGAGCTTAC |
| BAP049-hum15 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-hum16 HC | | |
| SEQ ID NO: 108 (Kabat) | HCDR1 | ACTTACTGGATGCAC |
| SEQ ID NO: 109 (Kabat) | HCDR2 | AATATTTATCCTGGTACTGGTGGTTCTAACTTC |
| | | GATGAGAAGTTCAAGAAC |
| SEQ ID NO: 110 (Kabat) | HCDR3 | TGGACTACTGGGACGGGAGCTTAT |
| SEQ ID NO: 111 (Chothia) | HCDR1 | GGCTACACATTCACCACTTAC |
| SEQ ID NO: 112 (Chothia) | HCDR2 | TATCCTGGTACTGGTGGT |
| SEQ ID NO: 110 (Chothia) | HCDR3 | TGGACTACTGGGACGGGAGCTTAT |
| BAP049-hum16 LC | | |
| SEQ ID NO: 113 (Kabat) | LCDR1 | AAGTCCAGTCAGAGTCTGTTAGACAGTGGAAAT |
| | | CAAAAGAACTTCTTGACC |
| SEQ ID NO: 114 (Kabat) | LCDR2 | TGGGCATCCACTAGGGAATCT |
| SEQ ID NO: 119 (Kabat) | LCDR3 | CAGAATGATTATAGTTATCCGTACACG |
| SEQ ID NO: 116 (Chothia) | LCDR1 | AGTCAGAGTCTGTTAGACAGTGGAAATCAAAAG |
| | | AACTTC |
| SEQ ID NO: 117 (Chothia) | LCDR2 | TGGGCATCC |
| SEQ ID NO: 120 (Chothia) | LCDR3 | GATTATAGTTATCCGTAC |
| BAP049-Clone-A HC | | |
| SEQ ID NO: 122 (Kabat) | HCDR1 | ACCTACTGGATGCAC |
| SEQ ID NO: 123 (Kabat) | HCDR2 | AACATCTATCCTGGCACCGGCGGCTCCAACTTC |
| | | GACGAGAAGTTCAAGAAC |
| SEQ ID NO: 124 (Kabat) | HCDR3 | TGGACAACCGGCACAGGCGCTTAT |
| SEQ ID NO: 125 (Chothia) | HCDR1 | GGCTACACCTTCACCACCTAC |
| SEQ ID NO: 126 (Chothia) | HCDR2 | TATCCTGGCACCGGCGGC |
| SEQ ID NO: 124 (Chothia) | HCDR3 | TGGACAACCGGCACAGGCGCTTAT |
| BAP049-Clone-A LC | | |
| SEQ ID NO: 127 (Kabat) | LCDR1 | AAGTCCTCCCAGTCCCTGCTGGACTCCGGCAAC |
| | | CAGAAGAACTTCCTGACC |
| SEQ ID NO: 128 (Kabat) | LCDR2 | TGGGCCTCCACCCGGGAATCT |
| SEQ ID NO: 129 (Kabat) | LCDR3 | CAGAACGACTACTCCTACCCCTACACC |
| SEQ ID NO: 130 (Chothia) | LCDR1 | TCCCAGTCCCTGCTGGACTCCGGCAACCAGAAG |
| | | AACTTC |
| SEQ ID NO: 131 (Chothia) | LCDR2 | TGGGCCTCC |
| SEQ ID NO: 132 (Chothia) | LCDR3 | GACTACTCCTACCCCTAC |
| BAP049-Clone-B HC | | |
| SEQ ID NO: 133 (Kabat) | HCDR1 | ACCTACTGGATGCAC |
| SEQ ID NO: 134 (Kabat) | HCDR2 | AATATCTACCCCGGCACCGGCGGCTCTAACTTC |
| | | GACGAGAAGTTTAAGAAT |
| SEQ ID NO: 135 (Kabat) | HCDR3 | TGGACTACCGGCACAGGCGCCTAC |
| SEQ ID NO: 136 (Chothia) | HCDR1 | GGCTACACCTTCACTACCTAC |
| SEQ ID NO: 137 (Chothia) | HCDR2 | TACCCCGGCACCGGCGGC |
| SEQ ID NO: 135 (Chothia) | HCDR3 | TGGACTACCGGCACAGGCGCCTAC |
| BAP049-Clone-B LC | | |
| SEQ ID NO: 138 (Kabat) | LCDR1 | AAATCTAGTCAGTCACTGCTGGATAGCGGTAAT |
| | | CAGAAGAACTTCCTGACC |
| SEQ ID NO: 139 (Kabat) | LCDR2 | TGGGCCTCTACTAGAGAATCA |
| SEQ ID NO: 140 (Kabat) | LCDR3 | CAGAACGACTATAGCTACCCCTACACC |
| SEQ ID NO: 141 (Chothia) | LCDR1 | AGTCAGTCACTGCTGGATAGCGGTAATCAGAAG |
| | | AACTTC |
| SEQ ID NO: 142 (Chothia) | LCDR2 | TGGGCCTCT |
| SEQ ID NO: 143 (Chothia) | LCDR3 | GACTATAGCTACCCCTAC |
| BAP049-Clone-C HC | | |
| SEQ ID NO: 122 (Kabat) | HCDR1 | ACCTACTGGATGCAC |
| SEQ ID NO: 123 (Kabat) | HCDR2 | AACATCTATCCTGGCACCGGCGGCTCCAACTTC |
| | | GACGAGAAGTTCAAGAAC |
| SEQ ID NO: 124 (Kabat) | HCDR3 | TGGACAACCGGCACAGGCGCTTAT |
| SEQ ID NO: 125 (Chothia) | HCDR1 | GGCTACACCTTCACCACCTAC |
| SEQ ID NO: 126 (Chothia) | HCDR2 | TATCCTGGCACCGGCGGC |
| SEQ ID NO: 124 (Chothia) | HCDR3 | TGGACAACCGGCACAGGCGCTTAT |
| BAP049-Clone-C LC | | |
| SEQ ID NO: 127 (Kabat) | LCDR1 | AAGTCCTCCCAGTCCCTGCTGGACTCCGGCAAC |
| | | CAGAAGAACTTCCTGACC |
| SEQ ID NO: 128 (Kabat) | LCDR2 | TGGGCCTCCACCCGGGAATCT |
| SEQ ID NO: 129 (Kabat) | LCDR3 | CAGAACGACTACTCCTACCCCTACACC |
| SEQ ID NO: 130 (Chothia) | LCDR1 | TCCCAGTCCCTGCTGGACTCCGGCAACCAGAAG |
| | | AACTTC |
| SEQ ID NO: 131 (Chothia) | LCDR2 | TGGGCCTCC |
| SEQ ID NO: 132 (Chothia) | LCDR3 | GACTACTCCTACCCCTAC |
| BAP049-Clone-D HC | | |
| SEQ ID NO: 122 (Kabat) | HCDR1 | ACCTACTGGATGCAC |
| SEQ ID NO: 144 (Kabat) | HCDR2 | AACATCTACCCTGGCACCGGCGGCTCCAACTTC |
| | | GACGAGAAGTTCAAGAAC |
| SEQ ID NO: 145 (Kabat) | HCDR3 | TGGACCACCGGAACCGGCGCCTAT |
| SEQ ID NO: 125 (Chothia) | HCDR1 | GGCTACACCTTCACCACCTAC |
| SEQ ID NO: 146 (Chothia) | HCDR2 | TACCCTGGCACCGGCGGC |
| SEQ ID NO: 145 (Chothia) | HCDR3 | TGGACCACCGGAACCGGCGCCTAT |
| BAP049-Clone-D LC | | |
| SEQ ID NO: 127 (Kabat) | LCDR1 | AAGTCCTCCCAGTCCCTGCTGGACTCCGGCAAC |
| | | CAGAAGAACTTCCTGACC |
| SEQ ID NO: 128 (Kabat) | LCDR2 | TGGGCCTCCACCCGGGAATCT |
| SEQ ID NO: 129 (Kabat) | LCDR3 | CAGAACGACTACTCCTACCCCTACACC |
| SEQ ID NO: 130 (Chothia) | LCDR1 | TCCCAGTCCCTGCTGGACTCCGGCAACCAGAAG |
| | | AACTTC |
| SEQ ID NO: 131 (Chothia) | LCDR2 | TGGGCCTCC |
| SEQ ID NO: 132 (Chothia) | LCDR3 | GACTACTCCTACCCCTAC |
| BAP049-Clone-E HC | | |
| SEQ ID NO: 133 (Kabat) | HCDR1 | ACCTACTGGATGCAC |
| SEQ ID NO: 134 (Kabat) | HCDR2 | AATATCTACCCCGGCACCGGCGGCTCTAACTTC |
| | | GACGAGAAGTTTAAGAAT |
| SEQ ID NO: 135 (Kabat) | HCDR3 | TGGACTACCGGCACAGGCGCCTAC |
| SEQ ID NO: 136 (Chothia) | HCDR1 | GGCTACACCTTCACTACCTAC |
| SEQ ID NO: 137 (Chothia) | HCDR2 | TACCCCGGCACCGGCGGC |
| SEQ ID NO: 135 (Chothia) | HCDR3 | TGGACTACCGGCACAGGCGCCTAC |
| BAP049-Clone-E LC | | |
| SEQ ID NO: 138 (Kabat) | LCDR1 | AAATCTAGTCAGTCACTGCTGGATAGCGGTAAT |
| | | CAGAAGAACTTCCTGACC |
| SEQ ID NO: 139 (Kabat) | LCDR2 | TGGGCCTCTACTAGAGAATCA |
| SEQ ID NO: 140 (Kabat) | LCDR3 | CAGAACGACTATAGCTACCCCTACACC |
| SEQ ID NO: 141 (Chothia) | LCDR1 | AGTCAGTCACTGCTGGATAGCGGTAATCAGAAG |
| | | AACTTC |
| SEQ ID NO: 142 (Chothia) | LCDR2 | TGGGCCTCT |
| SEQ ID NO: 143 (Chothia) | LCDR3 | GACTATAGCTACCCCTAC |

TABLE 2
Amino acid and nucleotide sequences of
the heavy and light chain framework
regions for humanized mAbs BAP049-hum01
to BAP049-hum16 and BAP049-Clone-A
to BAP049-Clone-E

| | Amino Acid | Nucleotide |
|---|---|---|
| | Sequence | Sequence |
| VHFW1 | EVQLVQSGAEV | GAAGTGCAGCTGGTGC |
| (type a) | KKPGESLRISC | AGTCTGGAGCAGAGGT |
| | KGS | GAAAAAGCCCGGGGAG |
| | (SEQ ID | TCTCTGAGGATCTCCT |
| | NO: 147) | GTAAGGGTTCT |
| | | (SEQ ID NO: 148) |
| | | GAAGTGCAGCTGGTGC |
| | | AGTCTGGCGCCGAAGT |
| | | GAAGAAGCCTGGCGAG |
| | | TCCCTGCGGATCTCCT |
| | | GCAAGGGCTCT |
| | | (SEQ ID NO: 149) |
| | | GAGGTGCAGCTGGTGC |
| | | AGTCAGGCGCCGAAGT |
| | | GAAGAAGCCCGGCGAG |
| | | TCACTGAGAATTAGCT |
| | | GTAAAGGTTCA |
| | | (SEQ ID NO: 150) |
| VHFW1 | QVQLVQSGAEV | CAGGTTCAGCTGGTGC |
| (type b) | KKPGASVKVSC | AGTCTGGAGCTGAGGT |
| | KAS | GAAGAAGCCTGGGGCC |
| | (SEQ ID | TCAGTGAAGGTCTCCT |
| | NO: 151) | GCAAGGCTTCT |
| | | (SEQ ID NO: 152) |
| VHFW2 | WVRQATGQGLE | TGGGTGCGACAGGCCA |
| (type a) | WMG | CTGGACAAGGGCTTGA |
| | (SEQ ID | GTGGATGGGT |
| | NO: 153) | (SEQ ID NO: 154) |
| | | TGGGTGCGACAGGCTA |
| | | CCGGCCAGGGCCTGGA |
| | | ATGGATGGGC |
| | | (SEQ ID NO: 155) |
| | | TGGGTCCGCCAGGCTA |
| | | CCGGTCAAGGCCTCGA |
| | | GTGGATGGGT |
| | | (SEQ ID NO: 156) |
| VHFW2 | WIRQSPSRGLE | TGGATCAGGCAGTCCC |
| (type b) | WLG | CATCGAGAGGCCTTGA |
| | (SEQ ID | GTGGCTGGGT |
| | NO: 157) | (SEQ ID NO: 158) |
| | | TGGATCCGGCAGTCCC |
| | | CCTCTAGGGGCCTGGA |
| | | ATGGCTGGGC |
| | | (SEQ ID NO: 159) |
| VHFW2 | WVRQAPGQGLE | TGGGTGCGACAGGCCC |
| (type c) | WMG | CTGGACAAGGGCTTGA |
| | (SEQ ID | GTGGATGGGT |
| | NO: 160) | (SEQ ID NO: 161) |
| VHFW3 | RVTITADKSTS | AGAGTCACGATTACCG |
| (type a) | TAYMELSSLRS | CGGACAAATCCACGAG |
| | EDTAVYYCTR | CACAGCCTACATGGAG |
| | (SEQ ID | CTGAGCAGCCTGAGAT |
| | NO: 162) | CTGAGGACACGGCCGT |
| | | GTATTACTGTACAAGA |
| | | (SEQ ID NO: 163) |
| | | AGAGTGACCATCACCG |
| | | CCGACAAGTCCACCTC |
| | | CACCGCCTACATGGAA |
| | | CTGTCCTCCCTGAGAT |
| | | CCGAGGACACCGCCGT |
| | | GTACTACTGCACCCGG |
| | | (SEQ ID NO: 164) |
| | | AGAGTGACTATCACCG |
| | | CCGATAAGTCTACTAG |
| | | CACCGCCTATATGGAA |
| | | CTGTCTAGCCTGAGAT |
| | | CAGAGGACACCGCCGT |
| | | CTACTACTGCACTAGG |
| | | (SEQ ID NO: 165) |
| VHFW3 | RFTISRDNSK | AGATTCACCATCTCCA |
| (type b) | NTLYLQMNSL | GAGACAATTCCAAGAA |
| | RAEDTAVYYC | CACGCTGTATCTTCAA |
| | TR | ATGAACAGCCTGAGAG |
| | (SEQ ID | CCGAGGACACGGCCGT |
| | NO: 166) | GTATTACTGTACAAGA |
| | | (SEQ ID NO: 167) |
| | | AGGTTCACCATCTCCC |
| | | GGGACAACTCCAAGAA |
| | | CACCCTGTACCTGCAG |
| | | ATGAACTCCCTGCGGG |
| | | CCGAGGACACCGCCGT |
| | | GTACTACTGTACCAGA |
| | | (SEQ ID NO: 168) |
| VHFW4 | WGQGTTVT | TGGGGCCAGGGCACCA |
| | VSS | CCGTGACCGTGTCCTC |
| | (SEQ ID | C |
| | NO: 169) | (SEQ ID NO: 170) |
| | | TGGGGCCAGGGCACCA |
| | | CAGTGACCGTGTCCTC |
| | | T |
| | | (SEQ ID NO: 171) |
| | | TGGGGTCAAGGCACTA |
| | | CCGTGACCGTGTCTAG |
| | | C |
| | | (SEQ ID NO: 172) |
| | | TGGGGCCAGGGCACAA |
| | | CAGTGACCGTGTCCTC |
| | | C |
| | | (SEQ ID NO: 173) |
| VLFW1 | EIVLTQSPDF | GAAATTGTGCTGACTC |
| (type a) | QSVTPKEKVT | AGTCTCCAGACTTTCA |
| | ITC | GTCTGTGACTCCAAAG |
| | (SEQ ID | GAGAAAGTCACCATCA |
| | NO: 174) | CCTGC |
| | | (SEQ ID NO: 175) |
| | | GAGATCGTGCTGACCC |
| | | AGTCCCCCGACTTCCA |
| | | GTCCGTGACCCCCAAA |
| | | GAAAAAGTGACCATCA |
| | | CATGC |
| | | (SEQ ID NO: 176) |
| VLFW1 | EIVLTQSPATL | GAAATTGTGTTGACAC |
| (type b) | SLSPGERATLS | AGTCTCCAGCCACCCT |
| | C | GTCTTTGTCTCCAGGG |
| | (SEQ ID | GAAAGAGCCACCCTCT |
| | NO: 177) | CCTGC |
| | | (SEQ ID NO: 178) |
| | | GAGATCGTGCTGACCC |
| | | AGTCCCCTGCCACCCT |
| | | GTCACTGTCTCCAGGC |
| | | GAGAGAGCTACCCTGT |
| | | CCTGC |
| | | (SEQ ID NO: 179) |
| | | GAGATCGTCCTGACTC |
| | | AGTCACCCGCTACCCT |
| | | GAGCCTGAGCCCTGGC |
| | | GAGCGGGCTACACTGA |
| | | GCTGT |
| | | (SEQ ID NO: 180) |
| VLFW1 | DIVMTQTPLSLP | GATATTGTGATGACCC |
| (type c) | VTPGEPASISC | AGACTCCACTCTCCCT |
| | (SEQ ID | GCCCGTCACCCCTGGA |
| | NO: 181) | GAGCCGGCCTCCATCT |
| | | CCTGC |
| | | (SEQ ID NO: 182) |
| VLFW1 | DWMTQSPLSLP | GATGTTGTGATGACTC |
| (type d) | VTLGQPASISC | AGTCTCCACTCTCCCT |
| | (SEQ ID | GCCCGTCACCCTTGGA |
| | NO: 183) | CAGCCGGCCTCCATCT |
| | | CCTGC |
| | | (SEQ ID NO: 184) |
| VLFW1 | DIQMTQSPSSL | GACATCCAGATGACCC |
| (type e) | SASVGDRVTIT | AGTCTCCATCCTCCCT |
| | C | GTCTGCATCTGTAGGA |
| | (SEQ ID | GACAGAGTCACCATCA |
| | NO: 185) | CTTGC |
| | | (SEQ ID NO: 186) |
| VLFW2 | WYQQKPGQAPR | TGGTACCAGCAGAAAC |
| (type a) | LLIY | CTGGCCAGGCTCCCAG |
| | (SEQ ID | GCTCCTCATCTAT |
| | NO: 187) | (SEQ ID NO: 188) |
| | | TGGTATCAGCAGAAGC |
| | | CCGGCCAGGCCCCCAG |
| | | ACTGCTGATCTAC |
| | | (SEQ ID NO: 189) |
| | | TGGTATCAGCAGAAGC |
| | | CCGGTCAAGCCCCTAG |
| | | ACTGCTGATCTAC |
| | | (SEQ ID NO: 190) |
| VLFW2 | WYQQKPGKAP | TGGTATCAGCAGAAAC |
| (type b) | KLLIY | CAGGGAAAGCTCCTAA |
| | (SEQ ID | GCTCCTGATCTAT |
| | NO: 191) | (SEQ ID NO: 192) |
| | | TGGTATCAGCAGAAGC |
| | | CCGGTAAAGCCCCTAA |
| | | GCTGCTGATCTAC |
| | | (SEQ ID NO: 193) |
| VLFW2 | WYLQKPGQ | TGGTACCTGCAGAAGC |
| (type c) | SPQLLIY | CAGGGCAGTCTCCACA |
| | (SEQ ID | GCTCCTGATCTAT |
| | NO: 194) | (SEQ ID NO: 195) |
| VLFW3 | GVPSRFSG | GGGGTCCCCTCGAGGT |
| (type a) | SGSGTDFT | TCAGTGGCAGTGGATC |
| | FTISSLEA | TGGGACAGATTTCACC |
| | EDAATYYC | TTTACCATCAGTAGCC |
| | (SEQ ID | TGGAAGCTGAAGATGC |
| | NO: 196) | TGCAACATATTACTGT |
| | | (SEQ ID NO: 197) |
| | | GGCGTGCCCTCTAGAT |
| | | TCTCCGGCTCCGGCTC |
| | | TGGCACCGACTTTACC |
| | | TTCACCATCTCCAGCC |
| | | TGGAAGCCGAGGACGC |
| | | CGCCACCTACTACTGC |
| | | (SEQ ID NO: 198) |
| | | GGCGTGCCCTCTAGGT |
| | | TTAGCGGTAGCGGTAG |
| | | TGGCACCGACTTCACC |
| | | TTCACTATCTCTAGCC |
| | | TGGAAGCCGAGGACGC |
| | | CGCTACCTACTACTGT |
| | | (SEQ ID NO: 199) |
| VLFW3 | GIPPRFSGS | GGGATCCCACCTCGAT |
| (type b) | GYGTDFTLT | TCAGTGGCAGCGGGTA |
| | INNIESEDA | TGGAACAGATTTTACC |
| | AYYFC | CTCACAATTAATAACA |
| | (SEQ ID | TAGAATCTGAGGATGC |
| | NO: 200) | TGCATATTACTTCTGT |
| | | (SEQ ID NO: 201) |
| VLFW3 | GVPSRFSGS | GGGGTCCCATCAAGGT |
| (type c) | GSGTEFTLT | TCAGCGGCAGTGGATC |
| | ISSLQPDDF | TGGGACAGAATTCACT |
| | ATYYC | CTCACCATCAGCAGCC |
| | (SEQ ID | TGCAGCCTGATGATTT |
| | NO: 202) | TGCAACTTATTACTGT |
| | | (SEQ ID NO: 203) |
| | | GGCGTGCCCTCTAGAT |
| | | TCTCCGGCTCCGGCTC |
| | | TGGCACCGAGTTTACC |
| | | CTGACCATCTCCAGCC |
| | | TGCAGCCCGACGACTT |
| | | CGCCACCTACTACTGC |
| | | (SEQ ID NO: 204) |
| VLFW3 | GVPSRFSGS | GGGGTCCCATCAAGGT |
| (type d) | GSGTDFTFT | TCAGTGGAAGTGGATC |
| | ISSLQPEDI | TGGGACAGATTTTACT |
| | ATYYC | TTCACCATCAGCAGCC |
| | (SEQ ID | TGCAGCCTGAAGATAT |
| | NO: 205) | TGCAACATATTACTGT |
| | | (SEQ ID NO: 206) |
| | | GGCGTGCCCTCTAGGT |
| | | TTAGCGGTAGCGGTAG |
| | | TGGCACCGACTTCACC |
| | | TTCACTATCTCTAGCC |
| | | TGCAGCCCGAGGATAT |
| | | CGCTACCTACTACTGT |
| | | (SEQ ID NO: 207) |
| VLFW4 | FGQGTKVEI | TTCGGCCAAGGGACCA |
| | K | AGGTGGAAATCAAA |
| | (SEQ ID | (SEQ ID NO: 209) |
| | NO: 208) | TTCGGCCAGGGCACCA |
| | | AGGTGGAAATCAAG |
| | | (SEQ ID NO: 210) |
| | | TTCGGTCAAGGCACTA |
| | | AGGTCGAGATTAAG |
| | | (SEQ ID NO: 211) |

TABLE 3
Constant region amino acid sequences of human IgG
heavy chains and human kappa light chain

| | |
|---|---|
| HC | IgG4 (S228P) mutant constant region amino acid |
| | sequence (EU Numbering) |
| | ASTKGPSVFP LAPCSRSTSE STAALGCLVK DYFPEPVTVS WNSGALTSGV |
| | HTFPAVLQSS GLYSLSSVVT VPSSSLGTKT YTCNVDHKPS NTKVDKRVES |
| | KYGPPCPPCP APEFLGGPSV FLFPPKPKDT LMISRTPEVT CVVVDVSQED |
| | PEVQFNWYVD GVEVHNAKTK PREEQFNSTY RVVSVLTVLH QDWLNGKEYK |
| | CKVSNKGLPS SIEKTISKAK GQPREPQVYT LPPSQEEMTK NQVSLTCLVK |
| | GFYPSDIAVE WESNGQPENN YKTTPPVLDS DGSFFLYSRL TVDKSRWQEG |
| | NVFSCSVMHE ALHNHYTQKS LSLSLGK (SEQ ID NO: 212) |
| LC | Human kappa constant region amino acid sequence |
| | RTVAAPSVFI FPPSDEQLKS GTASVVCLLN NFYPREAKVQ WKVDNALQSG |
| | NSQESVTEQD SKDSTYSLSS TLILSKADYE KHKVYACEVT HQGLSSPVTK |
| | SFNRGEC (SEQ ID NO: 213) |
| HC | IgG4 (S228P) mutant constant region amino acid sequence |
| | lacing C-terminal lysine (K) (EU Numbering) |
| | ASTKGPSVFP LAPCSRSTSE STAALGCLVK DYFPEPVTVS WNSGALTSGV |
| | HTFPAVLQSS GLYSLSSVVT VPSSSLGTKT YTCNVDHKPS NTKVDKRVES |
| | KYGPPCPPCP APEFLGGPSV FLFPPKPKDT LMISRTPEVT CVVVDVSQED |
| | PEVQFNWYVD GVEVHNAKTK PREEQFNSTY RVVSVLTVLH QDWLNGKEYK |
| | CKVSNKGLPS SIEKTISKAK GQPREPQVYT LPPSQEEMTK NQVSLTCLVK |
| | GFYPSDIAVE WESNGQPENN YKTTPPVLDS DGSFFLYSRL TVDKSRWQEG |
| | NVFSCSVMHE ALHNHYTQKS LSLSLG (SEQ ID NO: 214) |
| HC | IgG1 wild type |
| | ASTKGPSVFP LAPSSKSTSG GTAALGCLVK DYFPEPVTVS WNSGALTSGV |
| | HTFPAVLQSS GLYSLSSVVT VPSSSLGTQT YICNVNHKPS NTKVDKRVEP |
| | KSCDKTHTCP PCPAPELLGG PSVFLFPPKP KDTLMISRTP EVTCVVVDVS |
| | HEDPEVKFNW YVDGVEVHNA KTKPREEQYN STYRVVSVLT VLHQDWLNGK |
| | EYKCKVSNKA LPAPIEKTIS KAKGQPREPQ VYTLPPSREE MTKNQVSLIC |
| | LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 215) |
| HC | IgG1 (N297A) mutant constant region amino acid sequence |
| | (EU Numbering) |
| | ASTKGPSVFP LAPSSKSTSG GTAALGCLVK DYFPEPVTVS WNSGALTSGV |
| | HTFPAVLQSS GLYSLSSVVT VPSSSLGTQT YICNVNHKPS NTKVDKRVEP |
| | KSCDKTHTCP PCPAPELLGG PSVFLFPPKP KDTLMISRTP EVTCVVVDVS |
| | HEDPEVKFNW YVDGVEVHNA KTKPREEQYA STYRVVSVLT VLHQDWLNGK |
| | EYKCKVSNKA LPAPIEKTIS KAKGQPREPQ VYTLPPSREE MTKNQVSLIC |
| | LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 216) |
| HC | IgG1 (D265A, P329A) mutant constant region amino acid |
| | sequence (EU Numbering) |
| | ASTKGPSVFP LAPSSKSTSG GTAALGCLVK DYFPEPVTVS WNSGALTSGV |
| | HTFPAVLQSS GLYSLSSVVT VPSSSLGTQT YICNVNHKPS NTKVDKRVEP |
| | KSCDKTHTCP PCPAPELLGG PSVFLFPPKP KDTLMISRTP EVTCVVVAVS |
| | HEDPEVKFNW YVDGVEVHNA KTKPREEQYN STYRVVSVLT VLHQDWLNGK |
| | EYKCKVSNKA LAAPIEKTIS KAKGQPREPQ VYTLPPSREE MTKNQVSLIC |
| | LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 217) |
| HC | IgG1 (L234A, L235A) mutant constant region amino acid |
| | sequence (EU Numbering) |
| | ASTKGPSVFP LAPSSKSTSG GTAALGCLVK DYFPEPVTVS WNSGALTSGV |
| | HTFPAVLQSS GLYSLSSVVT VPSSSLGTQT YICNVNHKPS NTKVDKRVEP |
| | KSCDKTHTCP PCPAPEAAGG PSVFLFPPKP KDTLMISRTP EVTCVVVDVS |
| | HEDPEVKFNW YVDGVEVHNA KTKPREEQYN STYRVVSVLT VLHQDWLNGK |
| | EYKCKVSNKA LPAPIEKTIS KAKGQPREPQ VYTLPPSREE MTKNQVSLIC |
| | LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 218) |

TABLE 4
Amino acid sequences of the heavy
and light chain leader sequences
for humanized mAbs BAP049-Clone-A
to BAP049-Clone-E

| | | |
|---|---|---|
| BAP049- | HC | MEWSWVFLFF |
| Clone-A | | LSVTTGVHS |
| | | (SEQ ID NO: 219) |
| | LC | MSVPTQVLGL |
| | | LLLWLTDARC |
| | | (SEQ ID NO: 220) |
| BAP049- | HC | MAWVWTLPFL |
| Clone-B | | MAAAQSVQA |
| | | (SEQ ID NO: 221) |
| | LC | MSVLTQVLAL |
| | | LLLWLTGTRC |
| | | (SEQ ID NO: 222) |
| BAP049- | HC | MEWSWVFLFFL |
| Clone-C | | SVTTGVHS |
| | | (SEQ ID NO: 219) |
| | LC | MSVPTQVLGLL |
| | | LLWLTDARC |
| | | (SEQ ID NO: 220) |
| BAP049- | HC | MEWSWVFLFFL |
| Clone-D | | SVTTGVHS |
| | | (SEQ ID NO: 219) |
| | LC | MSVPTQVLGLL |
| | | LLWLTDARC |
| | | (SEQ ID NO: 220) |
| BAP049- | HC | MAWVWTLPFLM |
| Clone-E | | AAAQSVQA |
| | | (SEQ ID NO: 221) |
| | LC | MSVLTQVLALL |
| | | LLWLTGTRC |
| | | (SEQ ID NO: 222) |

### EXAMPLES

The Examples below are set forth to aid in the understanding of the inventions but are not intended to, and should not be construed to, limit its scope in any way.

### Example 1: Flat Dosing Schedules for the Anti-PD-1 Antibody Molecule

Based on pharmacokinetic (PK) modeling, utilizing flat dose is expected provide the exposure to patients at the appropriate Cmin concentrations. Over 99.5% of patients will be above EC50 and over 93% of patients will be above EC90. Predicted steady state mean Cmin for the exemplary anti-PD-1 antibody molecule utilizing either 300 mg once every three weeks (Q3W) or 400 mg once every four weeks (Q4W) is expected to be above 20 ug/mL (with highest weight, 150 kg) on average.

TABLE 5
Exemplary PK parameters based on flat dosing schedules

| | |
|---|---|
| Number of patients in PK dataset | 46 |
| CL (mL/h) | 10.9 [8.9, 13.2]; IIV: 62% |
| Exponent of Weight on CL | 0.54 [0.021, 1.06] |
| Volume of distribution at SS (L) | 7.2 [6.5, 7.9]; IIV: 22% |
| Half-Life (days) | 20 [17, 23]; IIV: 64% |
| Predicted Cmin (ug/mL) for 80 kg patient | 31 [22, 42] (400 mg q4w) |
| | 35 [26, 47] (300 mg q3w) |

The expected mean steady state Cmin concentrations for the exemplary anti-PD-1 antibody molecule observed with either doses/regimens (300 mg q3w or 400 mg q4w) will be at least 77 fold higher than the EC50 (0.42 ug/mL) and about 8.6 fold higher than the EC90. The ex vivo potency is based on IL-2 change in SEB ex-vivo assay.

Less than 10% of patients are expected to achieve Cmin concentrations below 3.6 ug/mL for either 300 mg Q3W or 400 mg Q4W. Less than 0.5% of patients are expected to achieve Cmin concentrations below 0.4 μg/mL for either 300 mg Q3W or 400 mg Q4W.

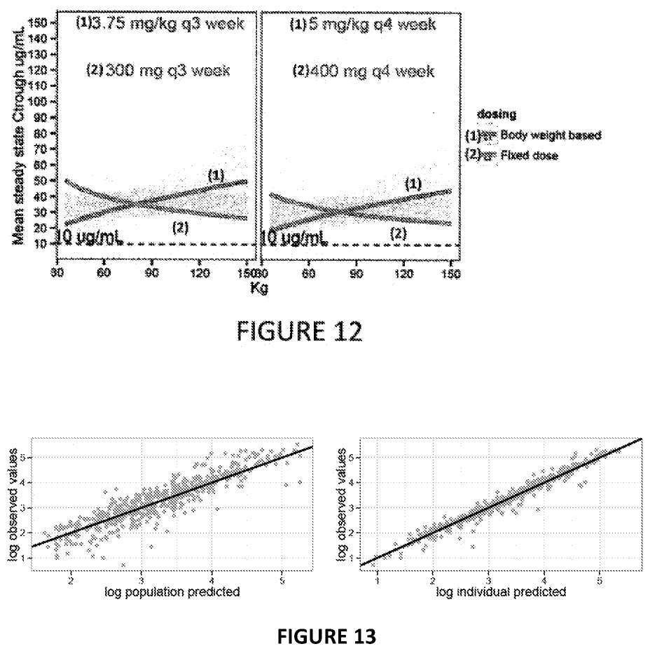

Predicted Ctrough (Cmin) concentrations across the different weights for patients while receiving the same dose of the exemplary anti-PD-1 antibody molecule are shown in FIG. 12. Body weight based dosing is compared to fixed dose (3.75 mg/kg Q3W vs. 300 mg Q3W and 5 mg/kg Q4W vs. 400 mg Q4W). FIG. 12 supports flat dosing of the exemplary anti-PD-1 antibody molecule.

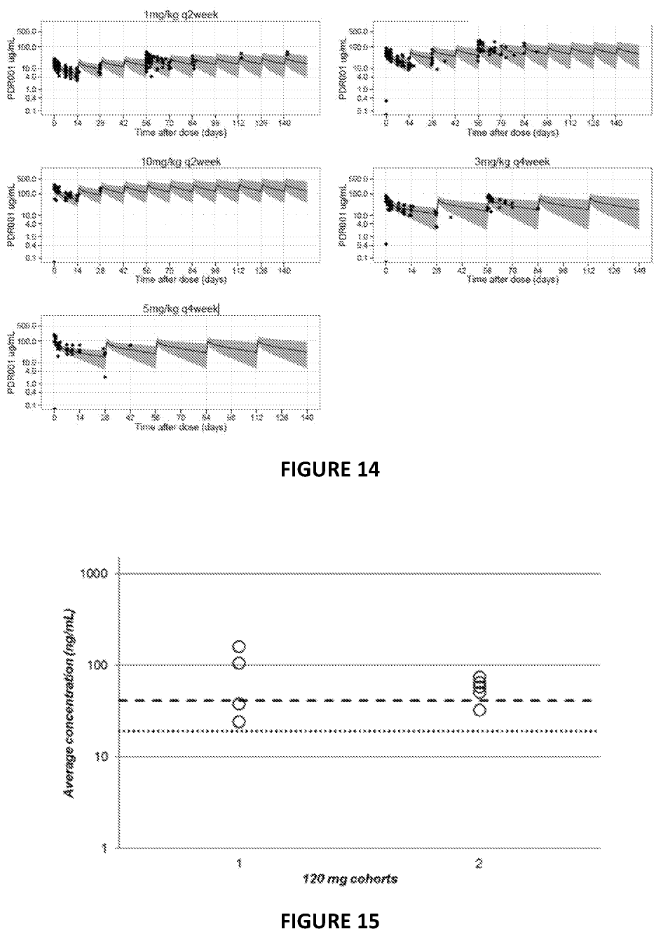

The PK model further is validated. As shown in FIG. 13, the observed versus model predicted concentrations lie on the line of unity. FIG. 14 shows that the model captures accumulation, time course, and within subject variability.

### Example 2: Dose and Dosing Regimen for HDM201

This example provides a summary of the clinical safety and pharmacokinetic (PK) data that supports the dose and regimen of the present invention for single agent HDM201 for patients with solid tumors in the phase 1 trial CHDM201X2101.

Herein, data are disclosed from this multicenter, open-label, first-in-human Phase I study of HDM201 in patients with TP53 wild-type (WT) advanced solid tumors, progressing on standard therapy or for which no standard therapy exists (NCT02143635).

The preferred was found to be 120 mg HDM201 given on dl and d8 of a 4 w cycle (regimen 1B). The data are from the monotherapy trial with a data cut-off date of 19 Sep. 2016.

The primary objective of the phase I part of the study is to determine the Maximum Tolerated Dose (MTD) and/or to identify the preferred dose of HDM201. The study design allowed parallel exploration of the safety, tolerability, and clinical activity of two broad dosing strategies for HDM201 across solid malignancies: intermittent high dose regimens (Regimen 1A and 1B) and extended low dose regimens (Regimen 2A and 2C). Table Ex2.1 summarizes the dosing regimens in each category that were evaluated in solid tumor patients. Table Ex2.2 provides the baseline characteristics of the patients involved in this study.

The endpoint for the primary objective is the incidence of Dose Limiting Toxicities (DLTs) during the first cycle of treatment. Although the primary analysis estimates the MTD based on DLT rate, the final preferred dose determination utilizes additional data beyond cycle 1 DLT rate, including later cycle tolerability, PK, PD and anti-tumor activity.

TABLE EX2.1
HDM201 Dosing regimens and dose levels
evaluated in solid tumor group

| | | | | Total |
|---|---|---|---|---|
| | | Dose levels | | number of |
| | Dosing Regimen | (number of patients) | | patients |
| Intermittent | 1A (d 1 Q3 weeks) | 12.5 mg | (n = 1) | N = 26 |
| high dose | | 25 mg | (n = 1) | |
| regimens | | 50 mg | (n = 4) | |
| | | 100 mg | (n = 4) | |
| | | 200 mg | (n = 5) | |
| | | 250 mg | (n = 6) | |
| | | 350 mg | (n = 5) | |
| | 1B (d 1, d 8 of | 120 mg | (n = 9) | N = 20 |
| | 4 w cycle) | 150 mg | (n = 8) | |
| | | 200 mg | (n = 3) | |
| Extended | 2A (2 weeks on/ | 1 mg | (n = 1) | N = 20 |
| low dose | 2 weeks off) | 2 mg | (n = 2) | |
| regimens | | 4 mg | (n = 4) | |
| | | 7.5 mg | (n = 4) | |
| | | 15 mg | (n = 4) | |
| | | 20 mg | (n = 5) | |
| | 2C (1 week on/ | 15 mg | (n = 8) | N = 19 |
| | 3 weeks off) | 20 mg | (n = 6) | |
| | | 25 mg | (n = 5) | |

Patient Population

Patients involved in this study are characterized by the following criteria:
Patients aged ≥18 years with a locally advanced or metastatic solid malignancy that had progressed despite standard therapy, or for which no effective standard therapy exists
Tumors with documented TP53 WT status (minimum of no mutations in exons 5-8) obtained from a tumor biopsy collected no longer than 36 months before screening
Measurable or non-measurable (but evaluable) disease as per Response Evaluation Criteria in Solid Tumors (RECIST) v1.1
Eastern Cooperative Oncology Group (ECOG) performance status ≤2
No prior treatment with compounds that inhibit the p53-HJDM2 interaction, e.g. RG7388 or NVP-CGM097
No treatment with growth factors targeting the myeloid lineage, e.g. G-CSF, ≤2 weeks prior to study treatment
Absolute neutrophil count >1,500/μL, platelet count >100,000/μL, hemoglobin >9.0 g/dL

Table Ex2.2 provides the baseline characteristics of the patients involved in this study.

TABLE EX2.2
Baseline characteristics (FAS)

| | Regimen 1A | | Regimen 1B | | Regimen 2A | | Regimen 2C | | All Regimens | |
|---|---|---|---|---|---|---|---|---|---|---|
| Characteristic | (n = 26) | | (n = 20) | | (n = 20) | | (n = 19) | | (N = 85) | |
| Age (median), years | 62 | | 63 | | 60 | | 57 | | 60 | |
| Range | 18-80 | | 31-78 | | 38-76 | | 37-74 | | 18-80 | |
| Sex (male), n (%) | 9 | (35) | 11 | (55) | 15 | (75) | 13 | (68) | 48 | (56) |
| Race, n (%) | | | | | | | | | | |
| Caucasian | 14 | (54) | 14 | (70) | 14 | (70) | 15 | (79) | 57 | (67) |
| Black | 1 | (4) | 0 | | 0 | | 0 | | 1 | (1) |
| Asian | 8 | (31) | 5 | (25) | 4 | (20) | 4 | (21) | 21 | (25) |
| Other | 2 | (8) | 1 | (5) | 2 | (10) | 0 | | 5 | (6) |
| Missing | 1 | (4) | 0 | | 0 | | 0 | | 1 | (1) |
| WHO/ECOG PS* | | | | | | | | | | |
| n (%) | | | | | | | | | | |
| 0 | 12 | (46) | 9 | (45) | 11 | (55) | 10 | (53) | 42 | (49) |
| 1 | 14 | (54) | 11 | (55) | 9 | (45) | 9 | (47) | 43 | (51) |
| Tumor type, n (%) | | | | | | | | | | |
| Liposarcoma | 3 | (12) | 4 | (20) | 1 | (5) | 1 | (5) | 9 | (11) |
| Sarcoma (others) | 8 | (31) | 2 | (10) | 6 | (30) | 3 | (16) | 19 | (22) |
| Skin melanoma | 0 | | 1 | (5) | 2 | (10) | 0 | | 3 | (4) |
| Uveal melanoma | 2 | (8) | 3 | (15) | 1 | (5) | 1 | (5) | 7 | (8) |
| Colon | 0 | | 1 | (5) | 4 | (20) | 3 | (16) | 8 | (9) |
| Kidney | 0 | | 0 | | 1 | (5) | 1 | (5) | 2 | (2) |
| Other | 13 | (50) | 9 | (45) | 5 | (25) | 10 | (53) | 37 | (44) |
| Number of prior | | | | | | | | | | |
| antineoplastic | | | | | | | | | | |
| regimens, n (%) | | | | | | | | | | |
| 0 | 0 | | 2 | (10) | 1 | (5) | 1 | (5) | 4 | (5) |
| 1 | 7 | (27) | 5 | (25) | 1 | (5) | 1 | (5) | 14 | (16) |
| 2 | 7 | (27) | 4 | (20) | 7 | (35) | 5 | (26) | 23 | (27) |
| ≥3 | 12 | (46) | 9 | (45) | 11 | (55) | 12 | (63) | 44 | (52) |

*WHO/ECOG PS: Eastern Cooperative Oncology Group/World Health Organization performance status

Statistical Analyses

Dose-escalation decisions were guided by the Bayesian logistic regression model (BLRMV) with the escalation with overdose control principle (EWOC).
Decisions were based on a synthesis of data available from all dose levels and regimens evaluated in the study including dose-limiting toxicities, all Common Terminology Criteria for Adverse Events (CTCAE) Grade ≥2 toxicity data during the first cycle of treatment, and pharmacokinetic and pharmacodynamic data from evaluable patients.
Cycle 2 hematological toxicities were also taken into account for dose escalation and regimen selection.

Dose/Regimen Justification

Of the 4 dosing regimens evaluated in solid tumors with single agent HDM201, the intermittent high dose regimen 1B (dl and d8 of 4 w cycle) were found to have the most favorable therapeutic index. Grade 3/4 thrombocytopenia was lowest in this regimen over all doses tested, and did not occur in patients treated at the selected RDE of 120 mg (see Table Ex2.3-1). The most frequent non-hematologic toxicities were gastrointestinal, but were not dose limiting at any of the dose levels evaluated across the 4 regimens. Pharmacokinetic data demonstrated that therapeutically relevant exposures were achieved at the 120 mg dose level for regimen 1B based on PK/PD modeling of preclinical data, and further supported by the observation of clinical efficacy in patients treated at this dose (1 patient with a long lasting PR, 1 patient with unconfirmed PR and 1 patient with SD). The 120 mg dose was also within the range of favorable doses recommended by the Bayesian logistic regression model (BLRM) supporting dose escalation. Therefore, regimen 1B at the dose of 120 mg was seen as most preferred dose and regimen.

Detailed Clinical Summary

At the time of data cut-off (19 Sep. 16), 85 patients with solid tumors have been treated with HDM201 across the 4 dosing regimens evaluated (see Table Ex2.1). Dose limiting toxicities across all regimens evaluated were primarily related to myelosuppression.

Of all dose-limiting cytopenias, grade 3/4 neutropenia and thrombocytopenia were most commonly observed across the regimens (Table Ex2.3). Therefore, the comparative incidence of grade 3/4 cytopenias (most importantly thrombocytopenia) across the 4 regimens was a key factor informing the selection of regimen and dose for expansion.

It was found that during the study that HDM201-induced myelosuppression can have delayed onset (beyond cycle 1). Therefore, dose limiting hematologic toxicities occurring in cycle 2 were also factored into dose escalation decisions during the course of the study, using a non-binding sensitivity model. Table Ex2.4 summarizes the number of dose limiting toxicities during cycle 1 and dose limiting hematologic toxicities in cycle 2 across all the regimens evaluated in solid tumors.

Intermittent high dose regimen 1A and extended low dose regimen 2A were the first to be evaluated in dose escalation. Both regimens had unfavorable rates of DLT and delayed hematologic toxicities at dose levels achieving predicted therapeutically relevant exposures. Therefore, cohorts exploring two additional regimens were opened: intermittent high dose regimen 1B and extended low dose regimen 2C. In the regimen 2C, DLTs were observed at dose levels at which exposures were below those predicted to be efficacious based on PK/PD modeling.

Twenty patients have been treated according to regimen 1B at 3 different dose levels (120 mg, 150 mg and 200 mg). The most frequent AEs (all grades) reported as suspected due to study treatment in regimen 1B were nausea (12 patients, 60.0%), thrombocytopenia/platelet count decreased (9 patients, 45.0%), neutropenia/neutrophil count decreased (8 patients, 40.0%) and vomiting (5 patients, 25.0%). Nine patients (45.0%) of this group experienced at least one CTCAE grade 3/4 AE suspected to be treatment-related. The three most frequent CTCAE grade 3/4 AEs considered suspected to study treatment were: neutropenia/neutrophil count decreased (6 patients, 30.0%), lipase increase (3 patients, 15%) and thrombocytopenia/platelet count decrease (2 patients, 10.0%). One event of prolonged neutropenia (onset on day 22 and lasting 18 days) meeting DLT criteria was observed in one patient treated at the dose of 150 mg. See Table Ex2.5 for further details. Of the 4 regimens evaluated, regimen 1B had the lowest overall incidence of grade 3/4 thrombocytopenia (Table Ex2.3).

At the preferred dose of 120 mg (regimen 1), there were no cases of grade 3/4 thrombocytopenia AEs (see Table Ex2.3-1). There were no dose interruptions or discontinuations due to thrombocytopenia at this dose level and no patients required platelet transfusions. The incidence of grade 3/4 neutropenia was similar across all regimens, and was observed in 2 out of 9 patients at the 120 mg dose level. There were no non-hematologic dose limiting toxicities or grade 3/4 AEs at this dose level.

Importantly, meaningful clinical activity was observed at the preferred dose of 120 mg (regimen 1). Of 9 patients treated at this dose, there was 1 PR (lasting 18 weeks and still ongoing at the cutoff date) in a patient with soft tissue sarcoma, 1 unconfirmed PR and 1 SD (lasting 8 weeks) both in patients with liposarcoma, indicating that therapeutically relevant exposures are achieved at this dose and schedule.

TABLE EX2.3
All cytopenia adverse events suspected to be study drug related - solid tumors

| | Neutropenia/ | | Leukopenia/white | | | | Thrombocytopenia/ | |
|---|---|---|---|---|---|---|---|---|
| | neutrophil count | | blood cell count | | | | platelet count | |
| | decreased* | | decreased* | | Anemia | | decreased* | |
| | All | | All | | All | | All | |
| | Grades | G3/4 | Grades | G3/4 | Grades | G3/4 | Grades | G3/4 |
| Regimen (n) | n(%) | n(%) | n(%) | n(%) | n(%) | n(%) | n(%) | n(%) |
| Regimen 1A | 9 (34.6) | 8 (30.7) | 9 (34.6) | 5 (19.2) | 10 (38.5) | 3 (11.5) | 12 (46.2) | 8 (30.8) |
| (n = 26) | | | | | | | | |
| Regimen 1B | 8 (40.0) | 6 (30.0) | 5 (25.0) | 1 (5.0) | 5 (25.0) | 0 | 9 (45.0) | 2 (10.0) |
| (n = 20) | | | | | | | | |
| Regimen 2A | 5 (25.0) | 4 (20.0) | 4 (20.0) | 3 (15.0) | 6 (30.0) | 4 (20.0) | 10 (50.0) | 7 (35.0) |
| (n = 20) | | | | | | | | |
| Regimen 2C | 3 (15.8) | 2 (10.5) | 2 (10.5) | 1 (5.3) | 4 (21.1) | 3 (15.8) | 8 (42.1) | 3 (15.8) |
| (n = 19) | | | | | | | | |
| RDE | 2 (22.2) | 2 (22.2) | 3 (33.3) | 0 | 2 (22.2) | 0 | 4 (44.4) | 0 |
| (Regimen | | | | | | | | |
| 1B 120 mg) | | | | | | | | |
| (n = 9) | | | | | | | | |

*includes combination of preferred terms

TABLE EX2.4
Treatment cycle 1 DLTs and Cycle 2 hematologic dose limiting toxicities in solid tumors

| | | | | DLTs | Hematologic dose limiting |
|---|---|---|---|---|---|
| | Dosing Regimen | Dose levels (n) | | (cycle 1) | toxicities (cycle 2) |
| Intermittent | 1A (d 1 Q3 weeks) | 12.5 mg | (n = 1) | 0 | 0 |
| high dose | | 25 mg | (n = 1) | 0 | 0 |
| regimens | | 50 mg | (n = 4) | 0 | 1 |
| | | 100 mg | (n = 4) | 0 | 0 |
| | | 200 mg | (n = 5) | 0 | 1 |
| | | 250 mg | (n = 6) | 0 | 1 |
| | | 350 mg | (n = 5) | 2 | 2 |

| | | | | | |
|---|---|---|---|---|---|
| Total (%) | N = 26 | 2 | (7.7%) | 5 | (19.2%) |

| | | | | |
|---|---|---|---|---|
| 1B (d 1, d 8 of | 120 mg | (n = 9) | 0 | 2 |
| 4 w cycle) | 150 mg | (n = 8) | 1 | 1 |
| | 200 mg | (n = 3) | 0 | Data not available at the |
| | | | | clinical cutoff |

| | | | | | |
|---|---|---|---|---|---|
| Total (%) | N = 20 | 1 | (5%) | 3 | (15%) |

| | | | | | |
|---|---|---|---|---|---|
| Extended | 2A (2 weeks on/ | 1 mg | (n = 1) | 0 | 0 |
| low dose | 2 weeks off) | 2 mg | (n = 2) | 0 | 0 |
| regimens | | 4 mg | (n = 4) | 0 | 0 |
| | | 7.5 mg | (n = 4) | 0 | 0 |
| | | 15 mg | (n = 4) | 0 | 1 |
| | | 20 mg | (n = 5) | 0 | 4 |

| | | | | | |
|---|---|---|---|---|---|
| Total (%) | N = 20 | 0 | (0%) | 5 | (25%) |

| | | | | |
|---|---|---|---|---|
| 2C (1 week on/ | 15 mg | (n = 8) | 0 | 1 |
| 3 weeks off) | 20 mg | (n = 6) | 0 | 0 |
| | 25 mg | (n = 5) | 2 | 0 |

| | | | | | |
|---|---|---|---|---|---|
| Total (%) | N = 19 | 2 | (10.5%) | 1 | (5.3%) |

TABLE EX2.5
All grades and grade 3/4 adverse events, suspected to be study drug
related, by preferred term and treatment - solid tumors - Regimen 1B

| | HDM201 1B | | HDM201 1B | | HDM201 1B | | | | | |
|---|---|---|---|---|---|---|---|---|---|---|
| | 120 mg | | 150 mg | | 200 mg | | All subjects | | | |
| | N = 9 | | N = 8 | | N = 3 | | N = 20 | | | |
| | All | Grade | All | Grade | All | Grade | All | | Grade | |
| | Grades | 3/4 | Grades | 3/4 | Grades | 3/4 | Grades | | 3/4 | |
| MEDDRA Preferred Term | n (%) | n (%) | n (%) | n (%) | n (%) | n (%) | n (%) | | n (%) | |
| Total | 9 (100) | 4 (44.4) | 7 (87.5) | 4 (50.0) | 3 (100) | 1 (33.3) | 19 | (95.0) | 9 | (45.0) |
| Nausea | 7 (77.8) | 1 (11.1) | 4 (50.0) | 0 | 1 (33.3) | 0 | 12 | (60.0) | 1 | (5.0) |
| Neutropenia | 2 (22.2) | 2 (22.2) | 4 (50.0) | 3 (37.5) | 0 | 0 | 6 | (30.0) | 5 | (25.0) |
| Anaemia | 2 (22.2) | 0 | 2 (25.0) | 0 | 1 (33.3) | 0 | 5 | (25.0) | 0 | |
| Diarrhoea | 3 (33.3) | 0 | 2 (25.0) | 0 | 0 | 0 | 5 | (25.0) | 0 | |
| Thrombocytopenia | 1 (11.1) | 0 | 4 (50.0) | 2 (25.0) | 0 | 0 | 5 | (25.0) | 2 | (10.0) |
| Vomiting | 3 (33.3) | 0 | 2 (25.0) | 0 | 0 | 0 | 5 | (25.0) | 0 | |
| Decreased Appetite | 1 (11.1) | 0 | 3 (37.5) | 0 | 0 | 0 | 4 | (20.0) | 0 | |
| Fatigue | 1 (11.1) | 0 | 2 (25.0) | 1 (12.5) | 1 (33.3) | 0 | 4 | (20.0) | 1 | (5.0) |
| Lipase Increased | 1 (11.1) | 0 | 2 (25.0) | 2 (25.0) | 1 (33.3) | 1 (33.3) | 4 | (20.0) | 3 | (15.0) |
| Platelet Count Decreased | 3 (33.3) | 0 | 1 (12.5) | 0 | 0 | 0 | 4 | (20.0) | 0 | |
| Abdominal Pain | 1 (11.1) | 0 | 2 (25.0) | 0 | 0 | 0 | 3 | (15.0) | 0 | |
| Neutrophil Count Decreased | 0 | 0 | 3 (37.5) | 2 (25.0) | 0 | 0 | 3 | (15.0) | 2 | (10.0) |
| White Blood Cell Count Decreased | 2 (22.2) | 0 | 1 (12.5) | 0 | 0 | 0 | 3 | (15.0) | 0 | |
| Asthenia | 1 (11.1) | 0 | 1 (12.5) | 0 | 0 | 0 | 2 | (10.0) | 0 | |
| Blood Creatine Phosphokinase | 2 (22.2) | 1 (11.1) | 0 | 0 | 0 | 0 | 2 | (10.0) | 1 | (5.0) |
| Increased | | | | | | | | | | |
| Blood Creatinine Increased | 1 (11.1) | 0 | 1 (12.5) | 0 | 0 | 0 | 2 | (10.0) | 0 | |
| Leukopenia | 1 (11.1) | 0 | 1 (12.5) | 1 (12.5) | 0 | 0 | 2 | (10.0) | 1 | (5.0) |
| Lymphopenia | 0 | 0 | 2 (25.0) | 1 (12.5) | 0 | 0 | 2 | (10.0) | 1 | (5.0) |

| | | | | | | | | | |
|---|---|---|---|---|---|---|---|---|---|
| Alanine Aminotransferase Increased | 0 | 0 | 1 (12.5) | 0 | 0 | 0 | 1 | (5.0) | 0 |
| Alopecia | 1 (11.1) | 0 | 0 | 0 | 0 | 0 | 1 | (5.0) | 0 |
| Amylase Increased | 0 | 0 | 0 | 0 | 1 (33.3) | 0 | 1 | (5.0) | 0 |
| Blood Bilirubin Increased | 0 | 0 | 1 (12.5) | 0 | 0 | 0 | 1 | (5.0) | 0 |
| Dehydration | 1 (11.1) | 0 | 0 | 0 | 0 | 0 | 1 | (5.0) | 0 |
| Dry Skin | 1 (11.1) | 0 | 0 | 0 | 0 | 0 | 1 | (5.0) | 0 |
| Dysgeusia | 1 (11.1) | 0 | 0 | 0 | 0 | 0 | 1 | (5.0) | 0 |
| Eye Pain | 0 | 0 | 1 (12.5) | 0 | 0 | 0 | 1 | (5.0) | 0 |
| Folliculitis | 0 | 0 | 1 (12.5) | 0 | 0 | 0 | 1 | (5.0) | 0 |
| Gamma-Glutamyltransferase | 0 | 0 | 1 (12.5) | 0 | 0 | 0 | 1 | (5.0) | 0 |
| Increased | | | | | | | | | |
| Headache | 0 | 0 | 1 (12.5) | 0 | 0 | 0 | 1 | (5.0) | 0 |
| Hyperkalaemia | 1 (11.1) | 0 | 0 | 0 | 0 | 0 | 1 | (5.0) | 0 |
| Hypocalcaemia | 1 (11.1) | 0 | 0 | 0 | 0 | 0 | 1 | (5.0) | 0 |
| Influenza Like Illness | 0 | 0 | 1 (12.5) | 0 | 0 | 0 | 1 | (5.0) | 0 |
| Lethargy | 0 | 0 | 1 (12.5) | 0 | 0 | 0 | 1 | (5.0) | 0 |
| Monocytosis | 1 (11.1) | 0 | 0 | 0 | 0 | 0 | 1 | (5.0) | 0 |
| Musculoskeletal Pain | 1 (11.1) | 0 | 0 | 0 | 0 | 0 | 1 | (5.0) | 0 |
| Myalgia | 1 (11.1) | 0 | 0 | 0 | 0 | 0 | 1 | (5.0) | 0 |
| Neuralgia | 0 | 0 | 1 (12.5) | 0 | 0 | 0 | 1 | (5.0) | 0 |
| Oedema | 0 | 0 | 0 | 0 | 1 (33.3) | 0 | 1 | (5.0) | 0 |
| Oral Candidiasis | 0 | 0 | 1 (12.5) | 0 | 0 | 0 | 1 | (5.0) | 0 |
| Pruritus | 0 | 0 | 1 (12.5) | 0 | 0 | 0 | 1 | (5.0) | 0 |
| Weight Decreased | 1 (11.1) | 0 | 0 | 0 | 0 | 0 | 1 | (5.0) | 0 |

Preferred terms are sorted in descending frequency of <all grades> column, as reported in the <All subjects> column.
A subject with multiple occurrences of an AE under one treatment is counted only once in the AE category For that treatment.
A subject with multiple adverse events is counted only once in the total row.
Only AEs occurring during treatment or within 30 days of the last study medication are reported.

Safety

Dose-limiting toxicities, typically occurring during Cycle 2, were neutropenia and thrombocytopenia.
Study drug-related all grade adverse events (AEs; occurring in >10% of all patients) are presented in Table Ex2.6.

TABLE EX2.6
Adverse Events Suspected To Be Study-drug Related, By Combined
Treatment Regimens (All Grades, Occurring in ≥10%)

All Regimens
(N = 85)

| Preferred Term, n (%) | All Grades | Grade 3/4 | |
|---|---|---|---|
| Nausea | 44 (52) | 1 | (1) |
| Thrombocytopenia | 27 (32) | 14 | (16) |
| Anemia | 25 (29) | 10 | (12) |
| Fatigue | 19 (22) | 2 | (2) |
| Decreased appetite | 19 (22) | 2 | (2) |
| Vomiting | 19 (22) | 0 | |
| Neutropenia | 18 (21) | 15 | (18) |
| Platelet count decreased | 15 (18) | 7 | (8) |
| Diarrhea | 13 (15) | 0 | |
| Leukopenia | 12 (14) | 8 | (9) |
| White blood cell count decrease | 11 (13) | 3 | (4) |

The most frequent non-hematologic toxicities were gastrointestinal, but were not dose-limiting at any of the dose levels evaluated across the 4 regimens; the most common all grade gastrointestinal AE was nausea (44/85; 52%), which was mostly mild to moderate in severity.
Study-drug related Grade 3/4 AEs of special interest are shown in Table Ex2.3. Grade 3/4 hematological toxicities suspected to be study-drug related were observed for all treatment regimens, occurring in up to ˜35% of patients. Grade 3/4 thrombocytopenia was lowest in Regimen 1B.

Clinical PK

Pharmacokinetic data have been evaluated throughout the course of the dose escalation. Two HDM201 drug variants have been evaluated during the course of the study (refer to the protocol for further details). Non-compartmental PK analysis showed a median time to reach maximum plasma concentrations ranging from 2.0 to 5.8 h across the dose range (2 to 350 mg). A preliminary dose proportionality assessment showed approximately dose proportional PK (AUClast and Cmax) over the dose range studied. For the majority of dose cohorts, the inter-patient variability (CV % Geo-mean) for AUClast and Cmax was low to moderate (6 to 58.5%). Furthermore, an integrated analysis of all available HDM201 concentrations was conducted using a population approach. The PK of HDM201 was best described by a 1-compartment PK model with a delayed zero- and first-order absorption process, and a linear clearance. Body weight was identified as a statistically significant covariate on apparent central volume of distribution (Vc/F), in which Vc/F increased with increasing body weight.

To further support the preferred dose for HDM201, compartmental PK modeling was used to estimate the individual average concentration per cycle for the 9 patients treated at 120 mg on regimen 1B (FIG. 15). For the majority of patients (7 out of 9), the estimated average drug concentrations per cycle were near or above the most conservative average tumor stasis concentration of ˜41 ng/mL per cycle determined from PKPD modeling of preclinical data (human SJSA-1 xenograft rat model).

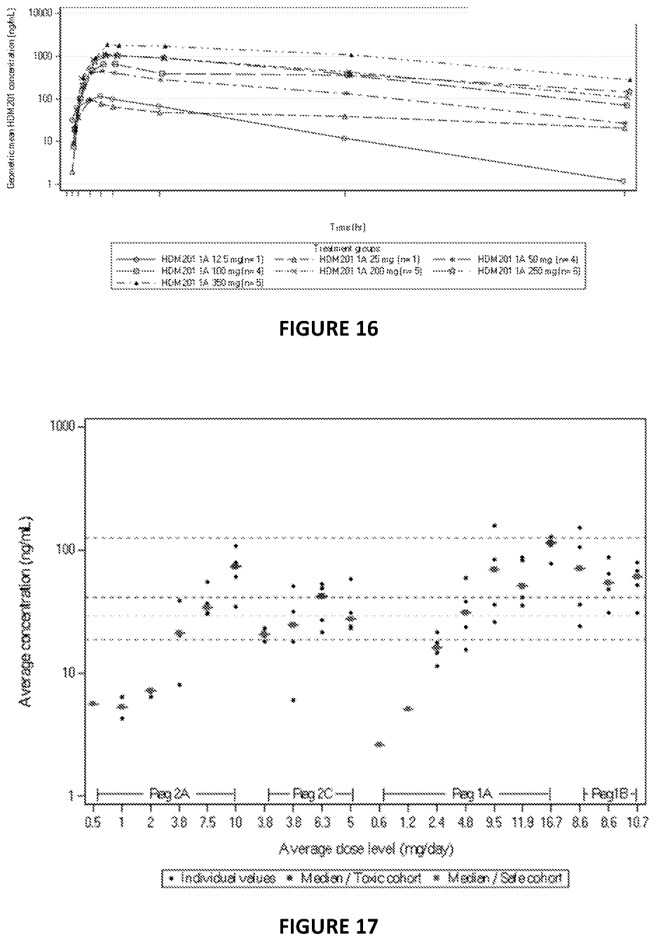

Representative geometric mean plasma concentration-time profiles for NVP-HDM201 after single dose (Day 1) for treatment Regimen 1A (12.5-350 mg) are presented in FIG. 16

Oral absorption was fast (median Tmax 2-5.8 hours) and did not vary by dose group (2-350 mg)

Mean plasma exposures (AUClast and Cmax) increased with increasing dose, with no major deviations from dose proportionality after single and repeated doses

NVP-HDM201 steady-state was generally reached by Day 8, with limited accumulation upon daily dosing

Median half-life estimated after Day 1 single dose (50-350 mg) ranged from 13.7 to 23.1 h

Inter-patient variability (CV % Geo-mean) in exposure was generally low to moderate.

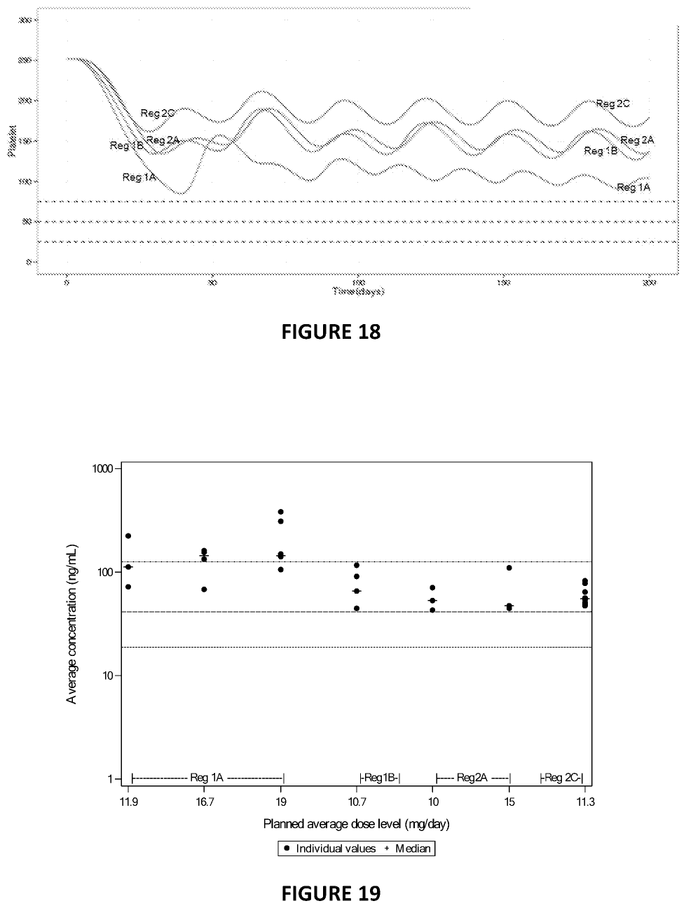

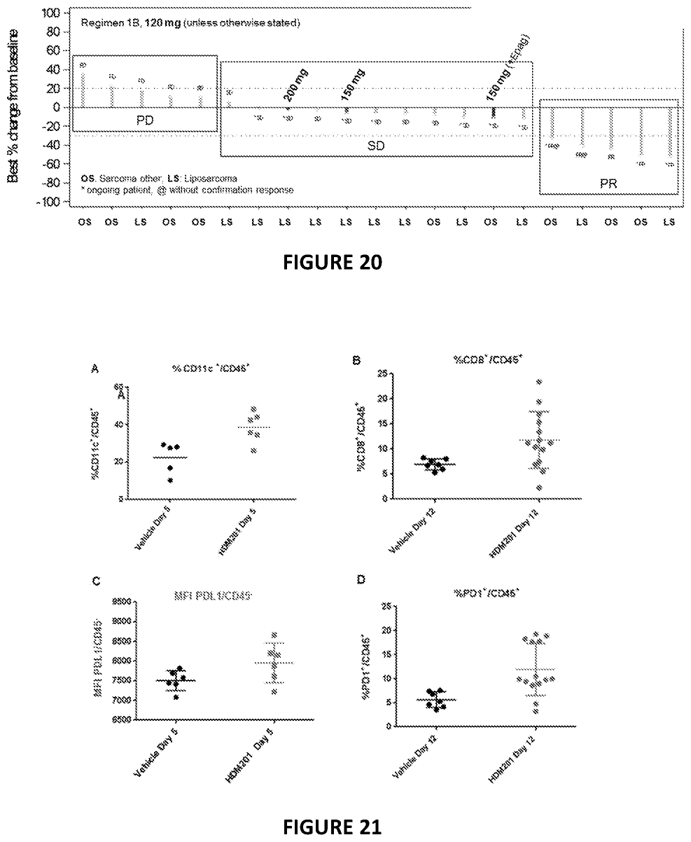

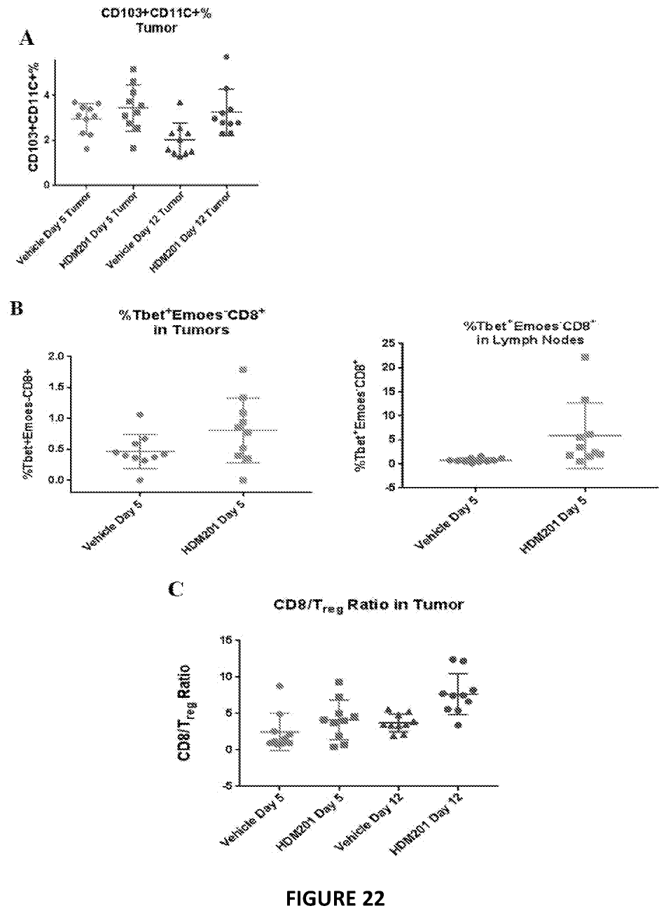

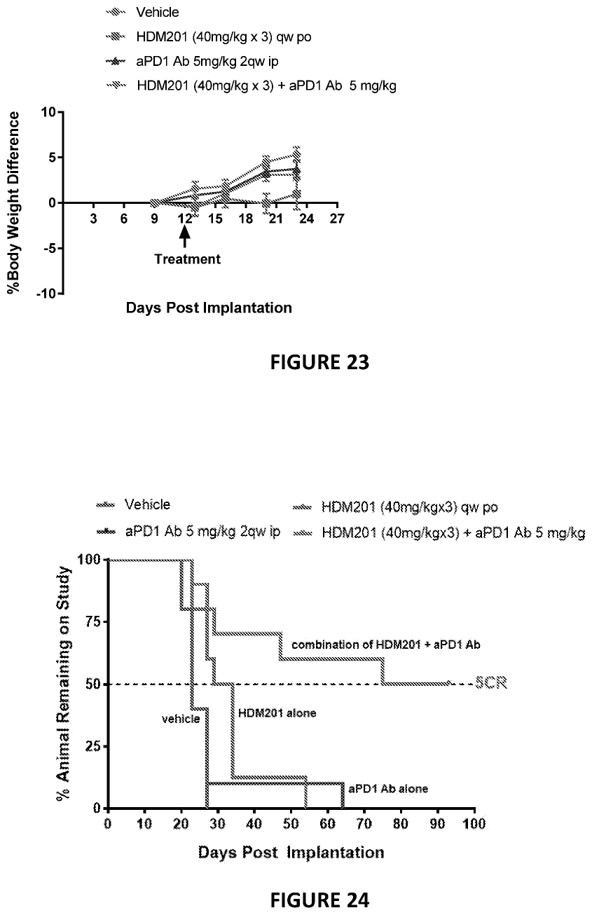

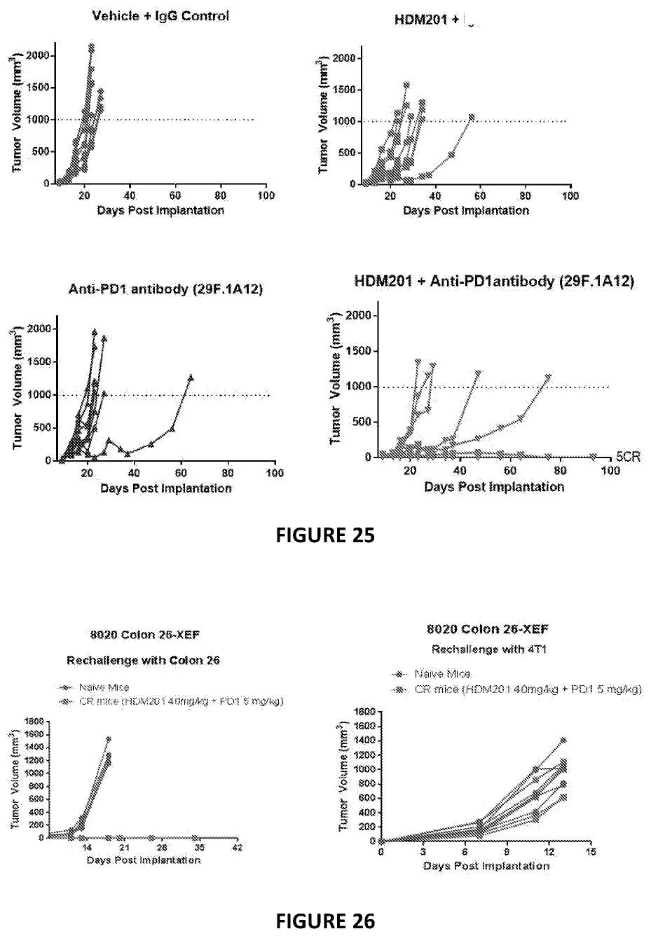

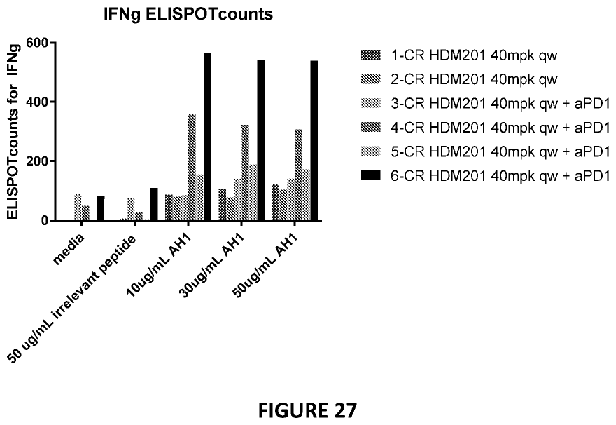

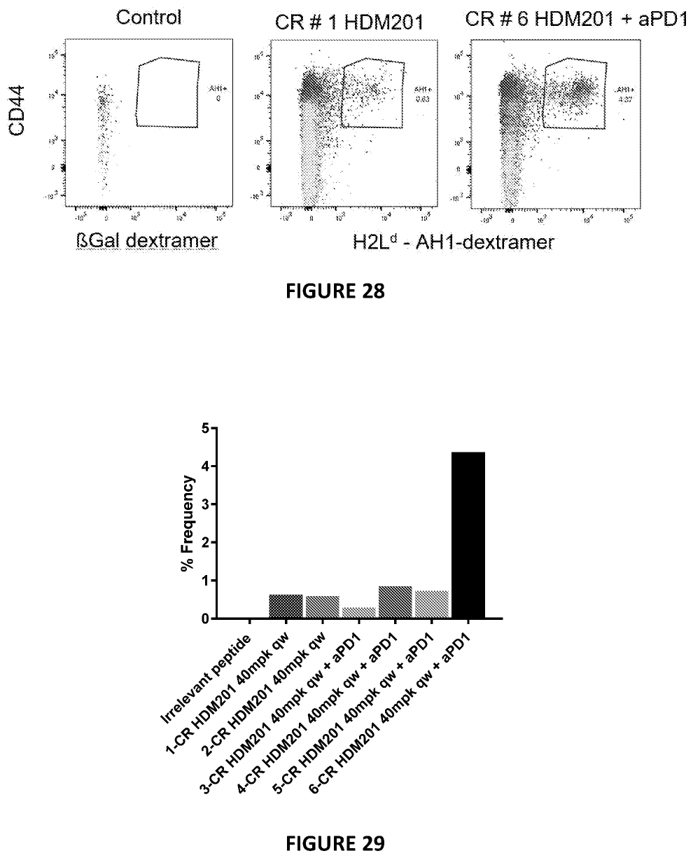

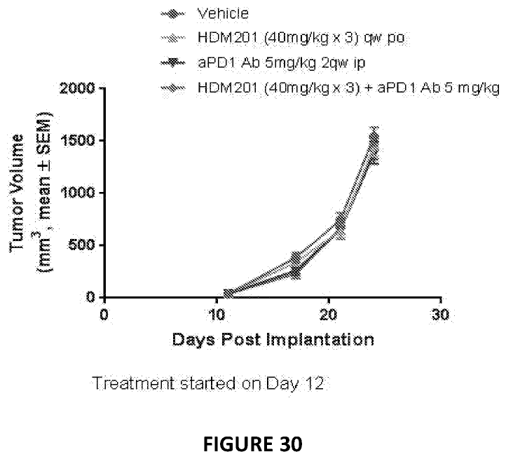

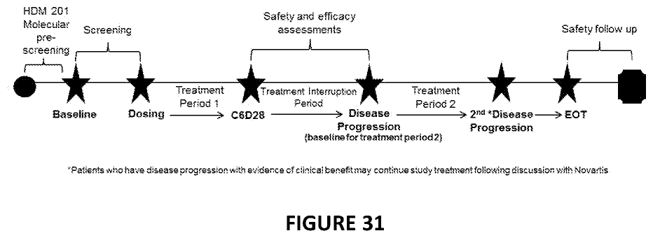

Compartmental population PK modeling of NVP-HDM201 was used to estimate the individual average plasma concentration for Cycle 1 and to allow comparison with preclinical average concentration for tumor stasis derived by PK/PD tumor growth modeling. The results are shown in FIG. 17.
Compared with Regimen 2A/2C, the average plasma concentration reached with Regimen 1A/1B was closer to the predicted preclinical target efficacious levels (125 ng/mL) required for 95% tumor regression (upper dashed line in FIG. 18) and near or above the estimated average concentrations for the most conservative average tumor stasis concentration of ≈41 ng/mL (dashed line) determined from PK/PD modeling of human SJSA-1 xenograft rat model (FIG. 17).
The dashed line at concentration of ≈19 ng/mL represents average tumor stasis determined from PK/PD modeling of preclinical data from a liposarcoma (HSAX2655) patient-derived xenograft rat model.
The dashed line at concentration 29.4 ng/mL represents IC50 value determined from the cellular activity in SJSA-1 cell line.

Statistical Analysis

This study utilizes a Bayesian logistic regression model (BLRM) to support dose escalation and estimate the MTD and/or determine the preferred dose for HDM201. The BLRM with escalation with overdose control (EWOC) enables incorporation of available prior information and updates the model parameters based upon new information about observed dose limiting toxicities (DLT) seen in the clinical study. During the course of the dose escalation for regimen 1A and 1B, DLT incidence has been used to update the model and support the decision for the next dose. When during the course of the study it became apparent that HDM201 induced bone marrow toxicity occurred predominantly during cycle 2, a non-binding sensitivity model including cycle 1 DLT and hematologic dose limiting AEs in cycle 2 (weighting all cytopenias equally) was used to guide dose escalation/RDE determinations. Additionally, decisions were at all times based on a synthesis of relevant data available from all dose levels evaluated in the study including low grade toxicities, PK, and PD data (when available) from evaluable patients.

The results of the BLRM using cycle 1 DLT events data from patients treated on regimen 1B (dose level 120 mg, 150 mg and 200 mg), supported escalation up to 400 mg HDM201. Median DLT rate at 120 mg was 3.5% and 25.7% as per protocol analysis and sensitivity analysis, respectively. Thus, 120 mg was found as preferred dose upon considering the lower incidence of clinically relevant grade 3/4 thrombocytopenia, manageable neutropenia, and the meaningful clinical activity observed at this dose.

Efficacy

At the time of data cut-off 2/46 (4%) patients receiving the high-dose intermittent regimens achieved PR (1 patient with STS-intimal sarcoma receiving Regimen 1A; 1 patient with

STS-hemangiopericytoma receiving Regimen 1B) (Table Ex2.7). 15/46 (33%) patients receiving the high-dose intermittent regimens and 14/39 (36%) patients receiving the low-dose extended regimens achieved SD (Table Ex2.7).

While meaningful disease control was observed in all dosing regimens (DCR: 34%), PRs were only seen in Regimens 1A and 1, suggesting that the high-dose intermittent regimens are more active.

By September 2017, strong antitumor efficacy had been observed for sarcoma patients (liposarcoma and other sarcomas). Out of 21 sarcoma patients treated with HDM201 according to regimen 1B, 5 patients showed partial response (PR), and 11 stable disease (SD). The disease only progressed (PD) in 5 patients (see FIG. 20).

TABLE EX2.7
Best Overall Response (FAS) (November 2016)

| | Regimen 1A | | Regimen 1B | | Regimen 2A | | Regimen 2C | |
|---|---|---|---|---|---|---|---|---|
| BOR, n (%) | (n = 26) | | (n = 20) | | (n = 20) | | (n = 19) | |
| CR | 0 | | 0 | | 0 | | 0 | |
| PR | 1 | (4) | 1 | (5) | 0 | | 0 | |
| SD | 8 | (31) | 7 | (35) | 7 | (35) | 7 | (37) |
| PD | 14 | (54) | 12 | (60) | 12 | (60) | 10 | (53) |
| Unknown | 3 | (12) | 0 | | 1 | (5) | 2 | (11) |
| ORR | 1 | (4) | 1 | (5) | 0 | | 0 | |
| 95% CI | 0.1-19.6 | | 0.1-24.9 | | 0.0-16.8 | | 0.0-17.6 | |
| DCR | 9 | (35) | 8 | (40) | 7 | (35) | 7 | (37) |

| | | | | |
|---|---|---|---|---|
| 95% CI | 17.2-55.7 | 19.1-63.9 | 15.4-59.2 | 16.3-61.6 |

BOR: best overall response; CI, confidence interval; CR: complete response; DCR: disease control rate (CR or PR or SD); FAS: full analysis set; ORR: overall response rate (CR or PR); PD: progressive disease; PR: confirmed partial response; SD: stable disease; BOR is based on investigator's assessment of disease status using RECIST 1.1; CR and PR are confirmed by repeat assessments performed not less than 4 weeks after the criteria for response is first met. The 95% CI is calculated using the exact (Clopper-Pearson) interval.

The median relative dose intensity (RDI) for patients with at least stable disease or better at the end of 32 weeks of treatment was similar in low-dose extended Regimens 2A and 2C. Of the 2 high-dose intermittent regimens, Regimen 1B had a more favorable RDI, supporting its overall better tolerability at therapeutically relevant doses (Table Ex2.8).

TABLE EX2.8
Relative Dose Intensity Summary For Patients With At Least
Stable Disease At The End Of 32 Weeks Of Treatment (SAS)

| Relative dose | | | | |
|---|---|---|---|---|
| intensity during | Regimen | Regimen | Regimen | Regimen |
| the first 32 weeks | 1A | 1B | 2A | 2C |
| of treatment | (n = 20) | (n = 20) | (n = 13) | (n = 19) |
| N | 11 (55) | 8 (40) | 7 (53.8) | 9 (47.4) |
| Median | 0.73 | 0.87 | 0.97 | 1 |
| Range | 0.33-1 | 0.5-1 | 0.72-1.42 | 0.61-1 |

SAS, safety analysis set.
n = total number of patients treated including only the treatment groups in the corresponding regimens:
Regimen 1A: ≥100 mg; Regimen 1B: ≥120 mg; Regimen 2A: ≥7.5 mg; Regimen 2C: ≥15 mg
N = number of patients with at least one SD or PR or CR or patients discontinued treatment for reasons other than PD.

PK/PD Model of Thrombocytopenia

Based on individual PK and platelet counts data over time a PK/PD model was established.

PK model: 1 compartment with biphasic absorption.
PD model: Adjusted Friberg model for thrombocytopenia including PLT transfusions and effect on HDM201 on proliferative cells and regulations.

### Data Base:

n=73 subjects
1301 PK observations
1023 PD platelets observations
427 PD GDF15 observations
The platelet kinetic profiles shown in FIG. 18 are modeled based on the following doses as tested in each regimen (in order from top to bottom in FIG. 18):
Reg2C (D1-7 Q4 wk): 25 mg ((25 mg×7 administration days)/28 days cycle=6.25 mg/day)
Reg2A (D1-14 Q4 wk): 20 mg ((20 mg×14 administration days)/28 days cycle=10 mg/day)
Reg1B (Days 1, 8 Q4 wk): 150 mg ((150 mg×2 admin. days)/28 days cycle=10.7 mg/day)
Reg1A (D1 Q3 wk): 350 mg ((350 mg×1 administration day)/21 days cycle=16.7 mg/day)

Based on this modeling, 1B has best overall platelet kinetic profile of the regimens that have demonstrated single agent activity.

The first occurrence of G4 thrombocytopenia with regimen 1B 150 mg in the clinical study occurred only after 100 days.

Addition of Eltrombopag to 1B could mitigate the relative delay and decreased peak of platelet recovery with subsequent cycles.

### Example 3: Pre-Clinical Investigations on the Combination of a PD-1 Inhibitor with the HDM2 Inhibitor HDM201

In this example, the effect of MDM2 inhibitor NVP-HDM201 (HDM201) on immune modulation in the Colon 26 colorectal adenocarcinoma (CRC) syngeneic mouse model is demonstrated. Using a multi-color FACS analysis, it was observed HDM201 increased number of CD103⁺CD11⁺ dendritic cells (DC) in the tumors at early time point (Day 5 post treatment), reflecting activation of DCs for antigen cross-presentation. HDM201 also increased the percentage of Tbet⁺EOMES-CD8⁺ T cells in the tumors as well as tumor draining lymph nodes; suggesting T cells were primed by DCs. At a later time point (Day 12 post treatment), increased CD8/T_{reg} ratio in the tumors was observed, indicating the induction of an effective immune response. In addition, HDM201 induced the upregulation of immune-suppressive proteins such as programmed death ligand 1 (PD-L1) on CD45⁻ cells and programed death-1 (PD1) in CD45⁺ T cells.

The anti-tumor effects of HDM201 as a monotherapy or in combination with an anti-PD1 antibody was assessed in the Colon 26 CRC syngeneic mouse model. HDM201 at 40 mg/kg inhibited tumor growth, while the addition of PD-1 blockade with an anti-PD1 antibody resulted in synergistic and durable tumor regression. The rate of complete tumor regression (CR) was significantly increased in the combination group (5 out of 10 CR) as compared to either treatment alone (no CR). This robust anti-tumor activity in the combination arm was consistent with the immune-modulation by HDM201, whereby the mice that achieved CR also developed long term specific memory against Colon 26 cells but not 4T1 cells. Taken together, these data demonstrated that MDM2 inhibition appears to modulate dendritic cell function, T cell priming, and CD8/T_{reg} ratio in the tumors, leading to tumor growth inhibition; combination with anti-PD1 antibody further released T cells from immunosuppressive state, and significantly improved the anti-tumor response. These data support the exploration of this combination in the clinic.

To investigate the immune-modulatory effects of HDM201, the Colon 26 murine CRC model was used, which was selected based on its wildtype p53 status. Our hypothesis being that inhibition of MDM2/p53 interaction will upregulate PDL1 in tumor cells and PD1 in lymphocytes, while blockade of the PD1/PDL1 interaction will potentiate the anti-tumor effects of HDM201.

Materials and Methods

### Materials

### Animals and Maintenance Conditions

For all experiments, animals were housed in a 12 hour (h) light/dark cycle facility and had access to food and water ad libitum. Animal characteristics are summarized in Table Ex3.1.

TABLE EX3.1
Animal Characteristics

| Species | Strain | Category | Vendor | Gender | Weight | Age |
|---|---|---|---|---|---|---|
| Mouse | Balb/c | Wild type | Jackson | Female | 18-25 g | 6-8 weeks |
| | | | Lab | | | |

### Statement on Animal Welfare

Animals were allowed to acclimate in the Novartis NIBR animal facility for at least 3 days prior to experimentation. Animals were handled in accordance with Novartis IACUC regulations and guidelines.

### Cells and Cell Culture Conditions

Syngeneic tumor models are mouse derived tumor cell lines implanted into animals of the same strain of mice from which the tumor was originated. This allows for the use of immunocompetent animals, which is central for testing of antibodies targeting immune cells used in these studies. Colon 26 is a Balb/c mouse colon carcinoma cell line induced by N-nitroso-N-methylurethane (Griswold D P and Corbett T H; A colon tumor model for anticancer agent evaluation Cancer 36:2441-2444, 1975). 4T1 is a spontaneously arising mammary tumor from Balb/c mice (Aslakson C J, Miller F R. Selective events in the metastatic process defined by analysis of the sequential dissemination of subpopulations of a mouse mammary tumor. Cancer Res. 52: 1399-1405, 1992).

Colon 26 cells were obtained from the Genomics Institute of the Novartis Research Foundation. 4T1 cells were purchased from ATCC. The master stocks for both cell lines were generated by the CLE (Cell Line Encyclopedia). Colon 26 and 4T1 cells were cultured in RPMI 1640 containing 10% heat-inactivated fetal bovine serum without antibiotics; the cells were free of mycoplasma and viral contamination in the IMPACT VIII PCR assay panel (IDEXX RADIL, IDEXX Laboratories INC, Westbrook, Me.).

### Compound Formulation and Antibody

HDM201-BB (succinic acid) was formulated in 0.5% w/v Methylcellulose (MC) solution in 50 mM phosphate buffer (pH 6.8) to a final concentration of 4.84 mg/ml (4 mg/ml free base). The salt/free base ratio is 1.21. The formulation was administered at 10 ml/kg, every 3 h for three times (3×q3h) on the first day of the week, with weekly (qw) administration by oral gavage (po). The formulation was stable for 3 weeks at 4° C. when protected from light.

An anti-PD1 antibody (Clone 29F.1A12, murine cross reactive) and its isotype control (Rat IgG2a) were purchased from BioLegend (San Diego, Calif., USA). Both antibodies were formulated to a final concentration of 0.5 mg/ml in PBS (Gibco, Life Technologies), and administered at a volume of 10 ml/kg by intraperitoneal injection (ip) twice a week (2 qw) for two weeks.

### Methods

### Colon 26 Syngeneic Tumor Model in Female Balb/c Mice.

Colon 26 cells were harvested at 80-95% confluence, washed, and re-suspended in cold PBS at a concentration of 2×10⁶ cells/ml. Finally, 0.2×10⁶ cells in a total volume of 100 μL were implanted subcutaneously (sc) into the upper right flank of naive Balb/c mice.

For Study 8020 Colon 26-XEF, animals were randomized and enrolled onto the study when tumor volumes reached a range of 27-60 mm³ on day 10 post cell implantation. All treatments were initiated three days later on day 13. For the PD studies, animals were randomized when the mean tumor volume reached 100˜120 mm³.

### Animal Monitoring

Animal well-being, behavior, and general health were monitored daily. Any moribund animals were euthanized.

Study Design

The designs of studies 7628 Colon 26-XPD, 8063 Colon 26-XPD and 8020 Colon 26-XEF including dose and schedule for treatment groups are summarized in Tables Ex3.2 to Ex3.4. Animals were weighed on dosing day(s) and the dosing volume was adjusted to body weight to 10 ml/kg. Tumor dimensions and body weights were recorded at the time of randomization and twice weekly thereafter for the study duration. The following data were collected after each day of data collection: incidence of mortality, individual and group average body weights, and individual and group average tumor volume.

TABLE EX3.2
Dose and Schedule for Study 7628 Colon 26-XPD

| | | | Time Points | |
|---|---|---|---|---|
| | | Number of | Post First | Sample |
| Groups | Treatment | Mice | Dose | Collection |
| 1 | Vehicle 10 ml/kg | 10 | Day 5 | Tumor, |
| | (3 × q3h) PO Day 0 | | | lymph node, |
| | | | | and spleen |
| 2 | HDM201 40 mg/kg | 10 | Day 5 | Tumor, |
| | (3 × q3h) PO Day 0 | | | lymph node, |
| | | | | and spleen |
| 3 | Vehicle 10 ml/kg | 10 | Day 12 | Tumor, |
| | (3 × q3h) PO Day 0, 7 | | | lymph node, |
| | | | | and spleen |
| 4 | HDM201 40 mg/kg | 10 | Day 12 | Tumor, |
| | (3 × q3h) PO Day 0, 7 | | | lymph node, |
| | | | | and spleen |

TABLE EX3.3
Dose and Schedule for Study 8063 Colon 26-XPD

| | | | Time Points | |
|---|---|---|---|---|
| | | Number of | Post First | Sample |
| Groups | Treatment | Mice | Dose | Collection |
| 1 | Vehicle 10 ml/kg | 8 | Day 5 | Tumor and |
| | (3 × q3h) PO Day 0 | | | spleen |
| 2 | HDM201 40 mg/kg | 8 | Day 5 | Tumor and |
| | (3 × q3h) PO Day 0 | | | spleen |
| 3 | Vehicle 10 ml/kg | 8 | Day 12 | Tumor and |
| | (3 × q3h) PO Day 0, 7 | | | spleen |
| 4 | HDM201 40 mg/kg | 8 | Day 12 | Tumor and |
| | (3 × q3h) PO Day 0, 7 | | | spleen |

TABLE EX3.4
Dose and Schedule for Study 8020 Colon 26-XEF

| | | Number of |
|---|---|---|
| Groups | Treatment | Mice |
| 1 | Vehicle 10 ml/kg (3 × q3h) PO Day 0, 7, 14 + | 10 |
| | Rat IgG2a 5 mg/kg IP Day 0, 4, 7, 10 | |
| 2 | HDM201 40 mg/kg (3 × q3h) PO Day 0, 7, 14 + | 10 |
| | Rat IgG2a 5 mg/kg IP Day 0, 4, 7, 10 | |
| 3 | Vehicle 10 ml/kg (3 × q3h) PO Day 0, 7, 14 + | 10 |
| | aPD1 Ab 5 mg/kg IP Day 0, 4, 7, 10 | |
| 4 | HDM201 40 mg/kg (3 × q3h) PO Day 0, 7, 14 + | 10 |
| | aPD1 Ab 5 mg/kg IP Day 0, 4, 7, 10 | |

Flow Cytometry Analysis

The tumor infiltrating lymphocytes (TILs) from tumors were analyzed by flow cytometry for both studies (7849 Colon 26-XPD and 8063 Colon 26-XPD). Lymph node lymphocytes were analyzed for 8063 Colon 26-XPD. The samples were plated into two separate 96 well plates, one for T cell staining (Table Ex3.5) and one for myeloid cell staining (Table Ex3.6).

TABLE EX3.5
Flow Cytometry Panels (7628 Colon 26-XPD)

| Panel | Marker | Clone | Fluorophore | Dilution |
|---|---|---|---|---|
| T Cells | CD45 | 30-F11 | BV510 | 1:200 |
| T Cells | CD11b/CD19 | 70/M1 | BV711 | 1:200 |
| T Cells | CD4 | GK1.5 | BV421 | 1:200 |
| T Cells | CD8 | 53-6.7 | BV650 | 1:200 |
| T Cells | FOXP3 | FJK-16s | APC | 1:100 |
| T Cells | PD-1 | 29F.1A12 | BV605 | 1:100 |
| T Cells | PD-L1 | 10F.9G2 | PE.Cy7 | 1:100 |
| T Cells | Live/Dead | Stain | Ef780 | 1:5000 |
| Myeloid cells | CD45 | 30-F11 | BV510 | 1:400 |
| Myeloid cells | CD11b | M1/70 | BV711 | 1:200 |
| Myeloid cells | CD11c | N418 | PE | 1:200 |
| Myeloid cells | Ly6C | HK1.4 | FITC | 1:200 |
| Myeloid cells | Ly6G | 1A8 | PacBlue | 1:200 |
| Myeloid cells | PD-L1 | 10F.9G2 | PE.Cy7 | 1:100 |
| Myeloid cells | PD-1 | 29F.1A12 | BV605 | 1:100 |
| Myeloid cells | Live/Dead | Stain | Ef780 | 1:5000 |

TABLE EX3.6
Flow Cytometry Panels (8063 Colon 26-XPD)

| Panel | Marker | Clone | Fluorophore | Dilution |
|---|---|---|---|---|
| T Cells | CD45 | 30-F11 | BV510 | 1:400 |
| T Cells | CD4 | GK1.5 | BUV395 | 1:200 |
| T Cells | CD8 | 53-6.7 | BV650 | 1:200 |
| T Cells | Foxp3 | FJK16s | AF488 | 1:100 |
| T Cells | T-bet | 4B10 | BV421 | 1:100 |
| T Cells | EOMES | Dan11Mag | PE.Cy7 | 1:100 |
| T Cells | TIM-3 | 5D12 | PE | 1:200 |
| T Cells | PD-1 | 29F.1A12 | BV605 | 1:200 |
| T Cells | PDL1 | 10F.9G2 | BV711 | 1:100 |
| T Cells | CD11b | M1/70 | BUV737 | 1:400 |
| T Cells | Live/Dead | Stain | Ef780 | 1:2000 |
| Myeloid Cells | CD45 | 30-F11 | BV785 | 1:500 |
| Myeloid Cells | CD11b | M1/70 | BUV737 | 1:1000 |
| Myeloid Cells | CD11c | N418 | APC-eFluor780 | 1:100 |
| Myeloid Cells | F480 | BM8 | APC | 1:100 |
| Myeloid Cells | I-A/I-E | M5/114.15.2 | BV650 | 1:400 |
| Myeloid Cells | Ly6C | HK1.4 | PECy7 | 1:500 |
| Myeloid Cells | Ly6G | 1A8 | BUV395 | 1:100 |
| Myeloid Cells | CD103 | 2.00E+07 | eFluor450 | 1:100 |
| Myeloid Cells | CD86 | Michel-17 | FITC | 1:100 |
| Myeloid Cells | CD40 | 1C10 | PerCP-eFluor710 | 1:100 |
| Myeloid Cells | PDL1 | 10F.9G2 | PE | 1:100 |
| Myeloid Cells | Live/Dead | Stain | Yellow | 1:1000 |

Tissue Processing

For Study 7628 Colon26-XPD, tumors and spleens were collected from mice on Day 5 and Day 12 post initiation of treatment. Single cell suspensions were generated according to RDS-2016-00163. Briefly, the tissues were minced with scissors followed by mechanical homogenization in dissociation buffer containing RPMI 1640 (Gibco, Life Technologies) with Liberase™ research grade collagenase (Roche) and DNase 1 recombinase (Roche) using the GentleMAX (Miltenyi). Following a 15 minute incubation at 37° C. in a water bath, the homogenates were quenched with 10% FBS and filtered on a 70 μM cell strainer (Falcon). At the end of this process, the single cell suspension of cells was obtained and 2 million cells were plated into 96-well plates for staining with either a T cell or myeloid cell panel of antibodies.

For Study 8063 Colon 26-XPD, tumors and lymph nodes were collected and then processed both mechanically and enzymatically into a single cell suspension according to RDS-2017-00141. The digestion process involves 4-5 consecutive digestion cycles with new digestion buffer containing DNase I (Roche), Collagenase P (Roche), and dispase (Gibco) in each cycle. At the end of this process, cell suspension was filtered on a 70 μM cell strainer to obtain single cell suspension. Two million cells were plated into 96 well plates for staining of T cell panel or myeloid cell panel antibodies.

FACS Staining and Data Acquisition

Once the cells were plated, the samples were stained with the live/dead staining as shown in Table Ex3.5 and Ex3.6. Following this, the samples were blocked with a 1:50 dilution of mouse Fc block (Miltenyi Biotec) for 30 minutes on ice. The samples were spun for 5 minutes at 1500 rpm and then stained with a fluorochrome-conjugated surface antibody mix as shown in Table Ex3.5 and Ex3.6 for 60 minutes. During the blocking and staining procedures, cells were maintained at 4° C. and protected from light.

For intracellular staining of T cells, after surface staining, the plates were spun again for 5 minutes at 1500 rpm, and then the cells were fixed and permeabilized overnight using a fix/perm kit (eBioscience). The cells were washed with a permeabilization buffer and then stained with the intracellular antibodies for 1 hour at 4° C. in the dark. The plates were washed twice in permeabilization buffer and suspended in 200 μl PBS. Data acquisition was performed using the LSRFortessa™ (BD Biosciences).

Data Analysis

### Body Weight

The percent change in body weight was calculated as (BW_{current}−BW_{D0})/(BW_{D0})×100%. Data was presented as mean percent body weight change from initial body weight measurement deemed Mean D₀±SEM. Do when referring to body weight correlates with measurements taken 7-10 days post tumor cell implant or 1-3 days prior of treatment initiation.

### Tumor Volume

Percent treatment/control (% T/C) and percent regression (% Reg) values were calculated using the following formulas, respectively:

% T/C=100×ΔT/ΔC if ΔT>0

% Reg=100×ΔT/Tᵢₙᵢₜᵢₐₗ if ΔT<0

where:
T=mean tumor volume of the drug-treated group on a given day of the study;
ΔT=mean tumor volume of the drug-treated group on a given day of the study−mean tumor volume of the drug-treated group on initial day of dosing;
Tᵢₙᵢₜᵢₐₗ=mean tumor volume of the drug-treated group on initial day of dosing;
C=mean tumor volume of the control group on final day of all the vehicle treated-mouse on study;
AC=mean tumor volume of the control group on final day of all the vehicle treated-mouse on study−mean tumor volume of the control group on initial day of dosing.

### Time to End Point

A Kaplan-Meier survival analysis was performed to compare differences in time to endpoint (TTE). Mice were scored as achieving tumor endpoint once tumor volume exceeded 1000 mm³ and scored as dead (“1”). Log-Rank (Mantel-Cox) survival analysis was performed (SigmaPlot13.0). Graphical analysis of median time to endpoint was performed in Prism (GraphPad v7).

### Flow Data Analysis

Analysis was performed after each run using FLOWJO v10.0.7 software from Treestar. For each analysis, the population of interest was gated to identify live leukocytes using a combination of morphological parameters (All cells: SSC-A vs FSC-A, single cells: SSC-H vs SSC-W; FSC-H vs FSC-W), and dead cell exclusion using eFluor780 (BD Biosciences) or yellow dye (Invitrogen). CD45⁺CD4⁺ and CD45⁺CD8⁺ labeling was used to gating T cells followed by CD4⁺Foxp3⁻ (T conventional), and CD4⁺ FoxP3⁺ (Treg) subsets. Tbet⁺EOMES⁻ cells were gated for newly primed T cells. Myeloid cells were gated according to published strategy by Broz and Krummel (Broz M L, Krummel M E. The emerging understanding of myeloid cells as partners and targets in tumor rejection Cancer Immunol Res. 2015 April; 3(4):313-9). Dendritic cells (DC) were gated for CD11b⁺CD11C⁺CD103⁺DCs. CD45⁻ specific labeling was used to identity non-lymphocytes including tumor cells, endothelial cells and fibroblasts.

### Statistical Analysis

For flow data, unpaired T-test and one way ANOVA were performed in SigmaPlot 13.0. Delta tumor volume and percent body weight difference were used for statistical analysis. Between groups comparisons were carried out using the ANOVA or Kruskal-Wallis ANOVA followed by a post hoc Tukey test. For time to end point analysis, Log-Rank (Mantel-Cox) survival analysis was performed (SigmaPlot 13.0). Graphical analysis of median time to endpoint was performed in Prism (GraphPad v7). For all statistical evaluations, the level of significance was set at p<0.05. Significance compared to the vehicle control group is reported unless otherwise stated.

Results

Pharmacodynamics: Immune Profiling (7628 Colon 26-XPD and 8063 Colon 26-XPD)

Immune profiling of TILs was performed by flow cytometry accordingly to the panel illustrated in Table Ex3.5 and Table Ex3.6. On Day 5 and Day 12 post first dose, animals were euthanized. Tumors, tumor draining lymph nodes and spleen were harvested for TIL characterization. Myeloid and T cell compartments from tumors and lymph nodes were enumerated and results are shown in FIGS. 21 and 22. Splenocytes were used mainly for staining controls (data not shown).

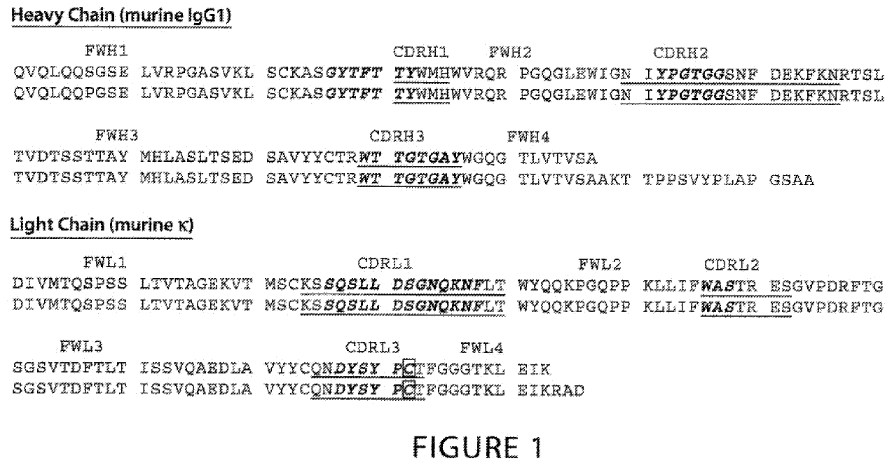

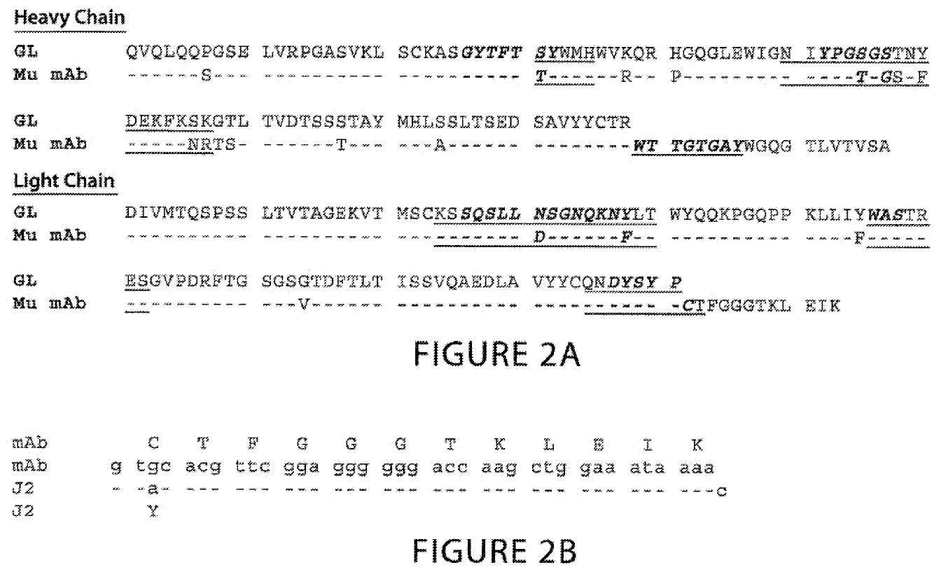

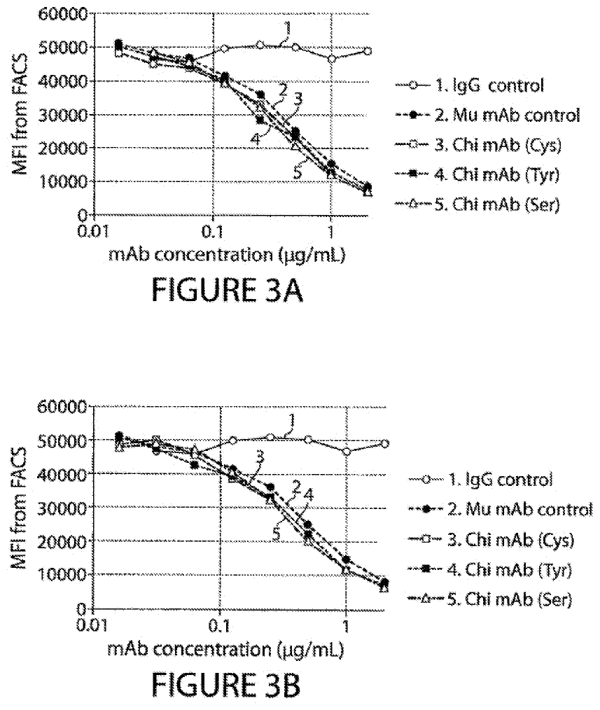

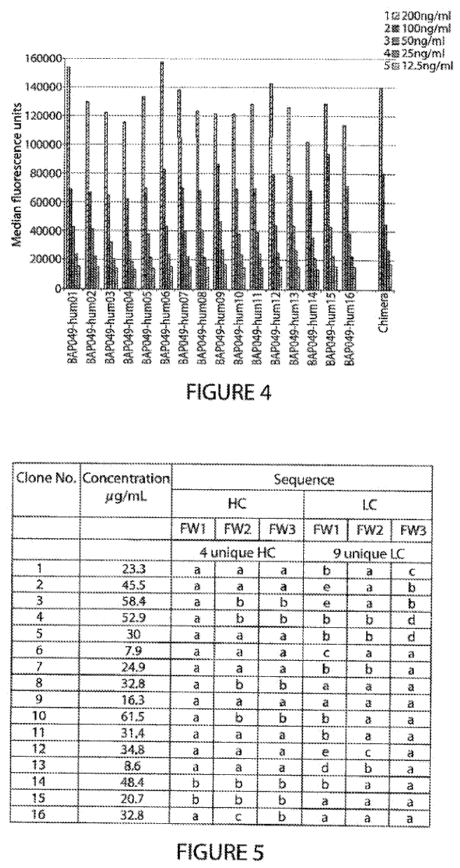

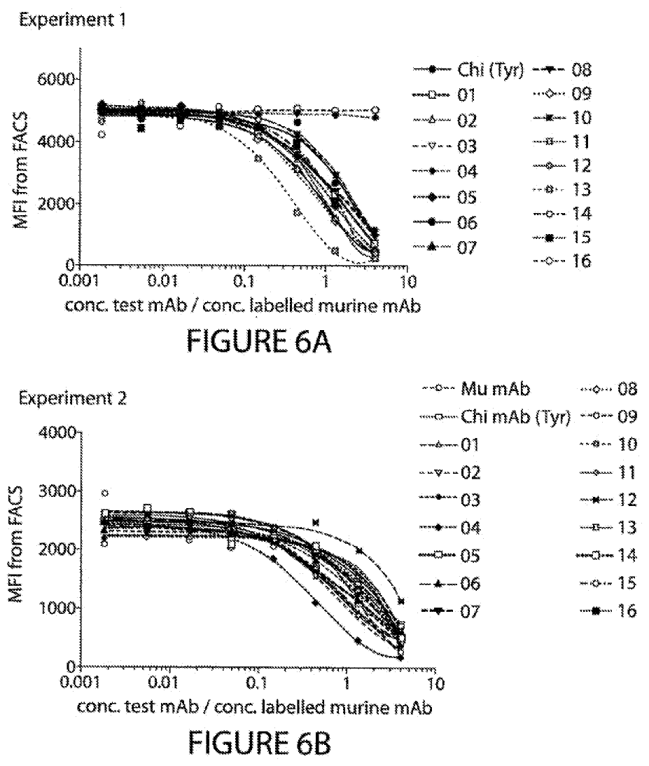

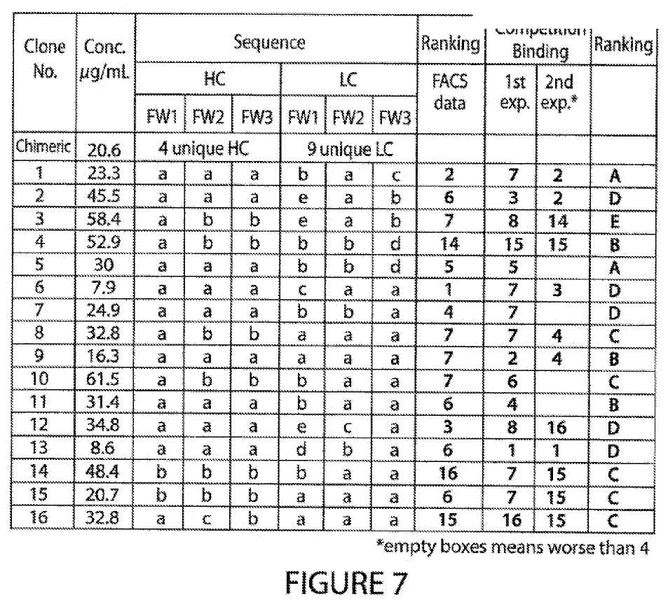

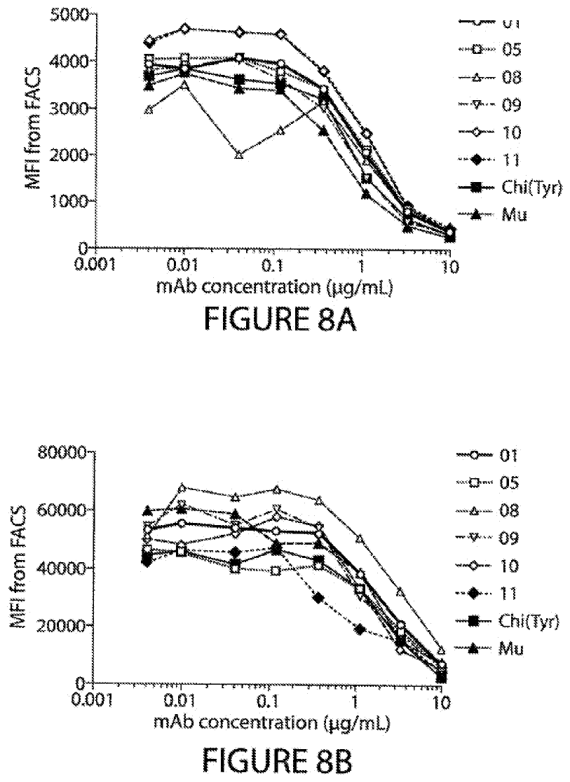

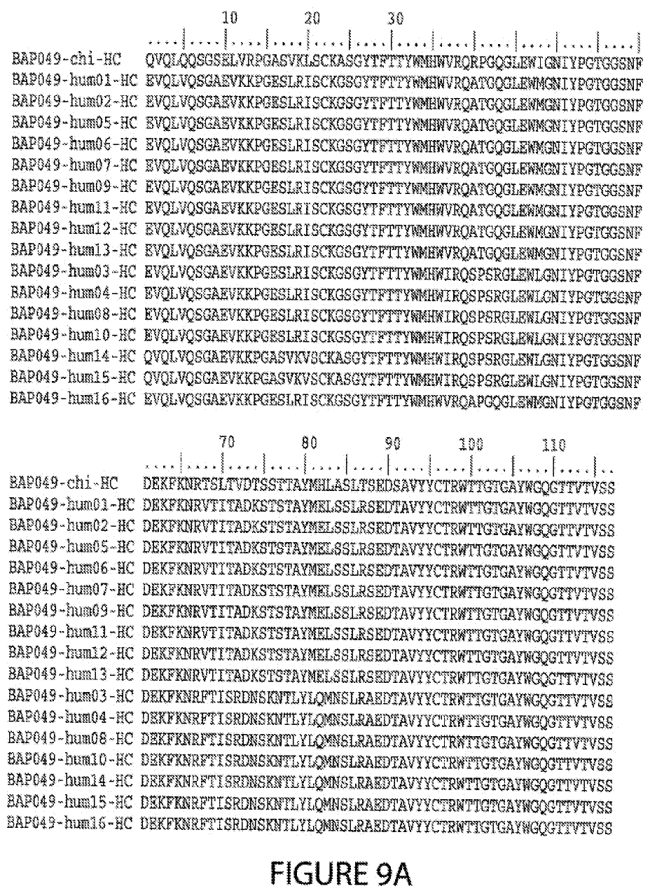

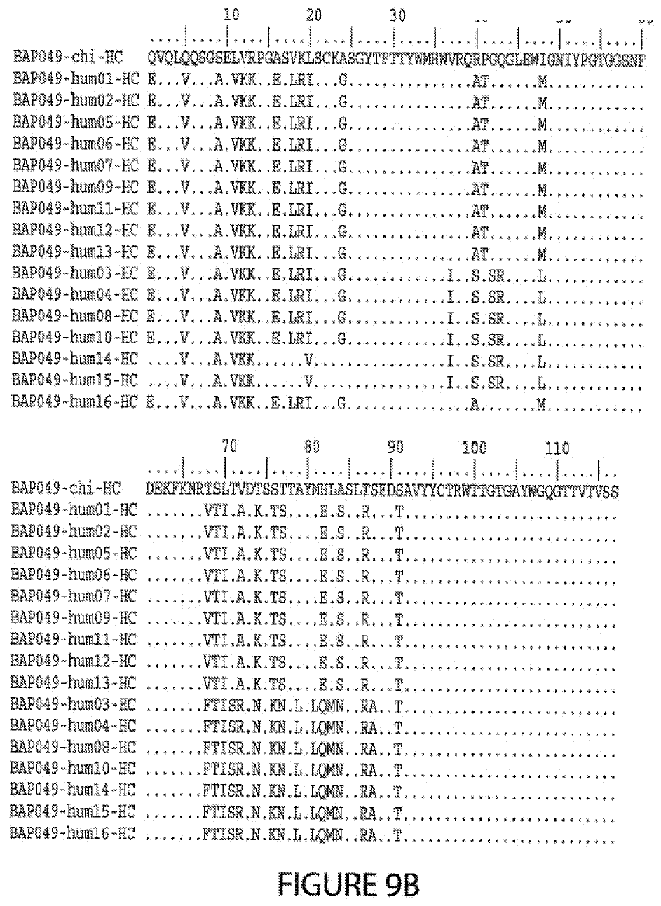

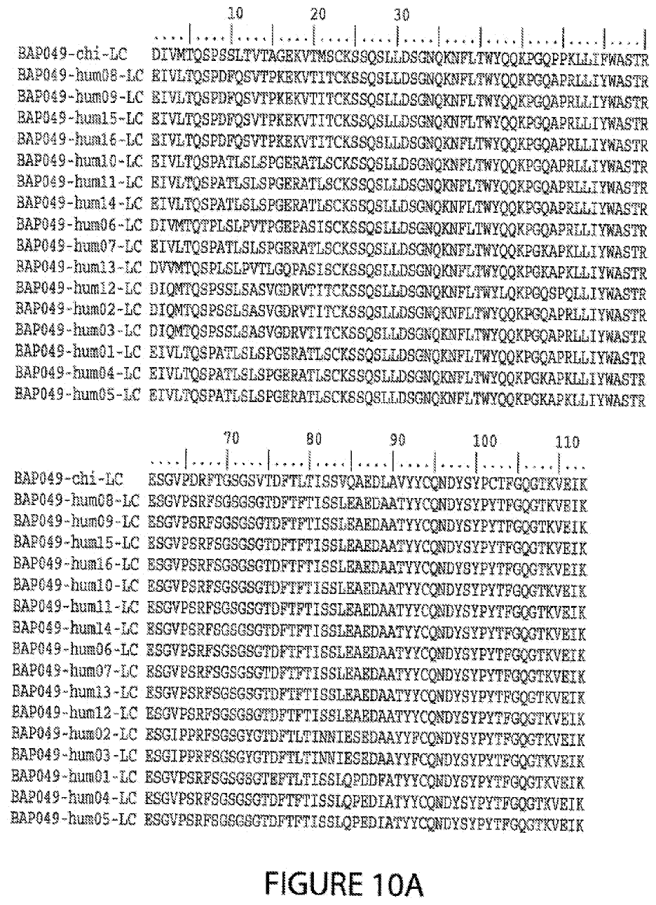

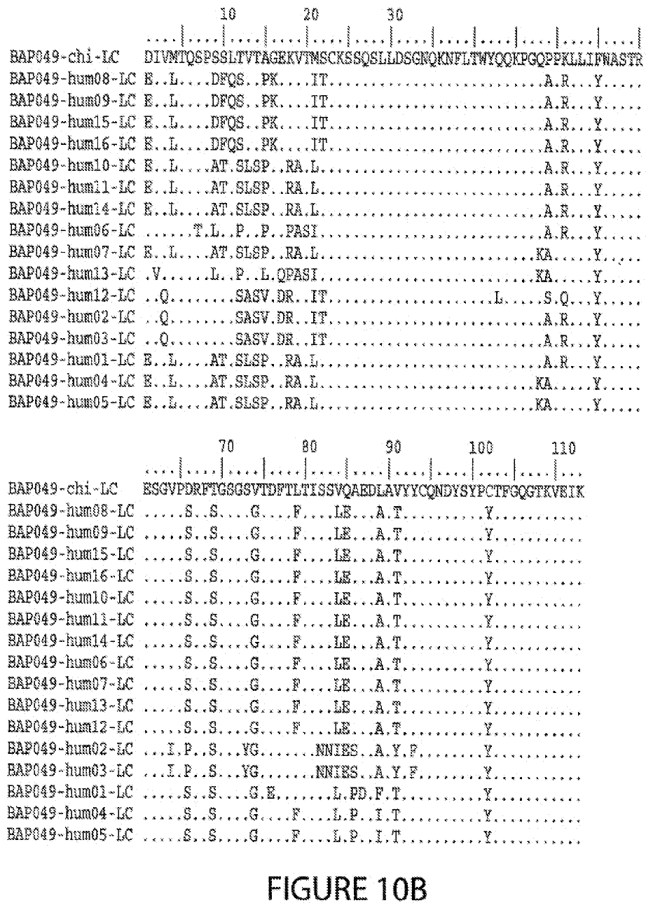

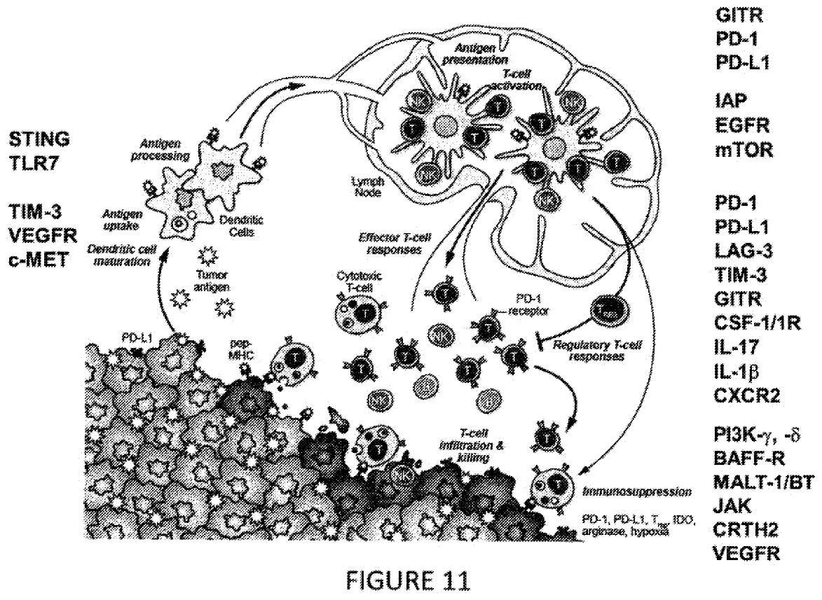

Initial immune profiling revealed HDM201 increased % CD11C+CD45+ cells and CD8 T cells (FIGS. 3-1). To further dissect the specific cell type regulated by HDM201, we performed a comprehensive FACS analysis. We found that HDM201 increased % CD103+CD11+ DCs, which are capable of antigen cross presentation; and increased newly primed % Tbet⁺EOMES⁻CD8⁺/CD45⁺ T cells, and the CD8/T_{reg} ratio (FIG. 22). In addition, HDM201 induced PDL1 expression in CD45⁻ cells shown as mean fluorescence intensity (MFI) of PDL1 in CD45⁻ populations (tumor cells, stroma cells or endothelial cells); HDM201 also increased % PD1⁺CD45⁺ cells (FIG. 21). These results indicated that HDM201 induced an active immune response against tumor; in the meantime, it triggered upregulation of immuno-suppressive proteins on immune cells as well as tumors cells.

Anti-Tumor Activity: Combination of HDM201 with aPD-1 Antibody in the Colon 26 Syngeneic Xenograft Tumor Model (8020 Colon 26-XEF)

The anti-tumor activity of HDM201 with aPD1 antibody targeting the PD-1/PD-L1 axis was explored in the Colon 26 murine syngeneic model (8020 Colon 26-XEF). Animals were randomized into treatment groups based on tumor volume on Day 9 post cell implantation. Treatments were initiated on Day 12, and continued with dosing of HDM201 every week for 3 weeks, and anti-PD1 antibody twice a week for 2 weeks. Animals remained on study until each reached individual endpoints, defined by tumor volume >1000 mm³. Tumor growth delay was assessed as median time to endpoint using the Kaplan-Meier analysis (GraphPad v7.0).

Tolerability

Animal body weight was monitored and reported as percent change relative to body weight prior to treatment (Day 9 post tumor implant). All treatments were well tolerated, as an increase in body weight was observed in all groups (FIG. 23). Day 23 post tumor implant was the last day that all animals remained on study and was therefore used for this analysis.

Anti-Tumor Activity

The median time to endpoint (TV≥1000 mm³) as determined by Kaplan-Meier (Log-Rank) analysis was used to assess treatment mediated tumor growth delay. As shown in Table Ex3.7, HDM201 as a monotherapy trended towards increasing the time to reach end point in comparison to the vehicle control, with a median time to endpoint of 31.5 days compared to 23 days, respectively. In contrast, blockade of PD1 resulted in time to endpoint of 23 days, which is the same as the vehicle group. Combination of HDM201 with aPD1 antibody significantly prolonged the time to endpoint to 84 days (p<0.05) (Table Ex3.7, FIG. 24).

TABLE EX3.7
Kaplan Meier Time to Endpoint (8020 Colon 26-XEF)

| | | | | Death | | Percent | Median Time to |
|---|---|---|---|---|---|---|---|
| Group | Treatment | Total | Missing | Events | Censored | Censored | Endpoint |
| 1 | Vehicle + IgG | 10 | 0 | 10 | 0 | 0 | 23 |
| 2 | HDM201 + IgG | 10 | 0 | 9 | 1 | 10 | 31.5 |
| 3 | Vehicle + | 10 | 0 | 10 | 0 | 0 | 23 |
| | aPD1 Antibody | | | | | | |
| 4 | HDM201 + | 10 | 0 | 5 | 5 | 50 | 84* |
| | aPD1 Antibody | | | | | | |

The individual animal tumor volume for each treatment group is shown in FIG. 25. Tumor growth was observed in all animals in the vehicle-treated group with all reached endpoint by Day 30. HDM201 as a monotherapy induced 1/10 animals having a partial response (FIG. 25); monotherapy anti-PD-1 antibody (clone #29F.1A12) also led to 1/10 animals exhibiting a partial response (FIG. 25). In contrast, the combination of anti-PD-1 antibody and HDM201 resulted in 2/10 animals exhibiting partial responses and 5/10 demonstrating complete responses (FIG. 25).

HDM201 Promotes Durable Tumor Specific Immune Response

Given the immuno-modulatory activity observed with HDM201 and its ability to combine with checkpoint blockade antibodies, the durability and specificity of the anti-tumor response that was generated was explored. In order to explore whether the anti-tumor response was antigen-specific, responder mice were re-challenged with Colon 26 on the left flank.

Those animals that achieved complete response were re-challenged (at day 123 post first cell implantation) with 0.2 million Colon 26 cells on the opposite of the flank, whereby all mice rejected the second injection of Colon 26 cells, while naive mice developed tumors (FIG. 26). In contrast, when re-challenged with 4T1 cells (at day 182), all mice developed tumors (similar to naïve mice), demonstrating that the memory is specific to Colon 26 cells (FIG. 26).

To further explore whether HDM201 treatment induced the development of anti-tumor memory T cell responses, splenocytes from responder mice were isolated and stimulated in vitro with CT26 associated antigen AH1 (gp70423-431) peptide (Huang et al 1996) and the number of IFN-γ producing cells were enumarated via ELISPOT assay. As shown in FIG. 27, antigen-specific production of IFN-γ by T cells were detected in all responders. Consistent with this, we observed an increase in frequency of AH1-specific CD8⁺ T cells in spleens of mice treated with HDM201 or combination of HDM201 with anti-PD1 antibody induced responders as detected by H2Ld-AH1 dextramers. (FIGS. 28 and 29). Overall, these data demonstrated that treatment with HDM201 promoted the development of durable tumor specific memory T cell responses.

In Vitro Characterization of p53 Knock Out Colon 26 Clones

p53 knock out Colon 26 Clones were grown in the presence of 1 μM HDM201 and screened for p53 expression by western blot, loading 40 μg total protein/sample, using an anti p53 antibody (Cell Signaling CST #2524). p53 negative clones were identified, grown without HDM201 for 4 days and then re-treated with 1 μM HDM201 for 24 hours, along with Colon26 parental cells, to monitor p53 pathway’ response. p53 and p21 changes were monitored by western blot and an 84 gene qPCR array was used to additionally confirm pathway activity (RT2 Profiler PCR Array p53 pathway, Cat No. 330231 PAMM-027ZA Qiagen). Select clones were also submitted for RNASeq analysis.

Using this p53 KO Colon26 model, it is shown that HDM201 is not able to inhibit tumor growth (FIG. 30). There was no additional benefit observed when the PD-1/PD-L1 axis was blocked (FIG. 30). Overall this data demonstrates the specificity of the anti-tumor activity of HDM201 as its beneficial response is only observed in p53 wild type tumors.

Conclusion

p53 is a transcription factor that plays a central role in guarding genomic stability of the cell through cell cycle arrest or induction of apoptosis. It has also been reported that p53 participates in the regulation of tumor immunity and in homeostatic regulation of immune responses. Here, it is demonstrated that HDM201 had an impact on immune cells in tumors as well as tumor draining lymph nodes. Specifically, HDM201 increased antigen presenting cells (DCs) in tumors, and draining lymph nodes. It is postulated that the DCs presented the tumor antigen to naive T cells, resulting in increased number of newly primed T cells in tumors as well as tumor draining lymph nodes. These T cells migrated to the tumor site, and recognized the tumor antigen to become activated. Ultimately, an increased CD8/T_{reg} ratio was observed in tumors. CD8 T cells are active effector cells which recognized tumor cells and induced tumor cell killing. In addition, it was observed PDL1 upregulation in CD45⁻ populations and the combination of HDM201 with anti-PD1 antibody significantly enhanced anti-tumor response compared to HDM201 and aPDL1 antibody as monotherapy. These results demonstrates that MDM2 inhibition triggered adaptive immunity which was further enhanced by blockade of PD-1/PD-L1 pathway in p53 wildtype tumor model, thereby providing a rationale for combining MDM2 inhibitors and checkpoint blocking antibodies in cancer patients with wildtype p53.

### Example 4: Clinical Investigations on the Combination of the PD-1 Inhibitor PDR001 (BAP049-Clone E, Spartalizumab) with the HDM2 Inhibitor HDM201

Clinical Trial

CPDR001X2102, EUDRACT number: 2016-000654-35
Phase Ib, open-label, multi-center study to characterize the safety, tolerability and pharmacodynamics (PD) of PDR001 in combination with (inter alia) HDM201

Rationale

The recent development of agents that enhance anti-tumor immunity is rapidly changing the treatment of cancer. However, these treatments are not effective in all cancer types, responses are often not durable, and many patients receive little or no benefit from treatment. Inhibitors of the PD-1/PD-L1 interaction are well tolerated and active across a remarkable range of cancer types, and will likely be one component of combination therapies that increase the response rate and durability of treatment.
The agents to be combined with PDR001 in this trial are used as immunomodulators, not as direct anti-tumor agents. The marketed agents, panobinostat and everolimus, will be used in indications where they are not approved, and in the case of everolimus will be administered at a significantly lower dose and less frequently than in the approved regimen. The goal is to use these agents to stimulate a more effective anti-tumor immune response, not as inhibitors of critical pathways that tumor cells depend upon for survival. For these reasons, and because enhancing the antitumor immune response is expected to be beneficial across many diseases, these combinations will be tested in indications that are different from those in which they are marketed.
With respect to PDR001 in combination with HDM201: HDM201, an inhibitor of the interaction between HDM2 and TP53, also enhances immune activation and efficacy of PD-1 blockade in preclinical models.
The study will identify the doses and schedule for further testing and will preliminarily assess the safety, tolerability, pharmacological and clinical activity of these combinations.
Following cancer types have been chosen for study:
Colorectal cancer (outside the mismatch repair-deficient sub-population): a cancer in which PD-1/PD-L1 therapy is ineffective for unknown reasons. Published data suggest that the immune context in tumors is prognostic and predictive of response to treatment with conventional chemotherapy, but for unknown reasons PD-1 or CTLA-4 inhibitors are ineffective (Kroemer G, Galluzzi L, Laurence Zitvogel L, et al. (2015) Colorectal cancer: the first neoplasia found to be under immunosurveillance and the last one to respond to immunotherapy? OncoImmunology 4:7, e1058597-1-3). The purpose of including CRC is to learn whether combination therapy may activate a more effective anti-tumor response.
Patients with MSS CRC will be eligible for PDR001+HDM01 arm, as this disease has a relatively low rate of TP53 mutation.
Renal cell carcinoma, for PDR001+HDM201 arm only: The purpose of including RCC is to provide a preliminary assessment of whether combination therapy with HDM201 may broaden activity, deepen responses, or lead to more durable responses. The diseases for study with PDR001+HDM201 will be modified to reflect the necessity of identifying only patients with TP53 wild-type disease for eligibility.
Renal cell carcinoma has a low rate of TP53 mutation and a minority of patients respond to treatment with PD-1 inhibitors.
The purpose of the study is to provide preliminary evidence that a combination may increase the response rate and durability of response compared with published data for treatment with single agent PD-1 inhibitors. Each disease group may include a subset of patients previously treated with PD-1 checkpoint inhibitors to explore whether combination therapy might overcome resistance to PD-1 blockade. For each disease, no specific molecular selection will be applied as the data available at present generally do not support excluding patients on the basis of approved molecular diagnostic tests such as PD-L1 expression.
This study will explore whether these agents can be safely combined with PDR001 and if so, will identify the doses and regimens appropriate for further study. The study will also assess whether each combination induces pharmacologic changes in tumor that would suggest potential clinical benefit, and will preliminarily assess the efficacy of each combination.

Objectives

### Primary Objectives

=>To characterize the safety and tolerability of PDR001 in combination with HDM201 to identify recommended doses and schedules for future studies

### Endpoints:

### Safety

Frequency and severity of treatment-emergent AEs and SAEs
Changes between baseline and post-baseline laboratory parameters and vital signs Escalation only
Incidence of dose limiting toxicities (DLTs) during the first two cycles of treatment

### Tolerability

Frequency of dose interruptions and reductions
Dose intensities

### Key Secondary Objective

=>To characterize changes in the immune infiltrate in tumors
Endpoints: Histopathology of Tumor Infiltrating Lymphocytes (TILs) by Hematoxylin and eosin (H&E) stain, characterization of TILs and myeloid cell infiltrate by IHC (such as CD8, FoxP3 and myeloid markers as appropriate)

### Secondary Objectives

=>To estimate the anti-tumor activity of PDR001 in combination with HDM201
Endpoints: Best overall response (BOR), PFS per irRC and RECIST v1.1. Treatment Free Survival (TFS)
=>To characterize the pharmacokinetics of all study drugs
Endpoints: Serum concentration of PDR001 and PK parameters, Plasma concentrations of HDM201 and PK parameters
=>To assess immunogenicity of PDR001
Endpoints: Presence and/or concentration of anti-PDR001 antibodies

### Exploratory Objectives

=>Estimate the anti-tumor activity of PDR001 in combination with HDM201 following the re-administration of study treatment

### Endpoint: BOR per RECIST v1.1

Study Design

This is a phase Ib, multi-center, open-label study of PDR001 in combination with HDM201 in patients with TP53 wildtype MSS-CRC or RCC.
The study is comprised of a dose escalation part followed by a dose expansion part with eleven investigational arms.
During the dose escalation part of the study, patients will be treated with a fixed dose of PDR001, administered i.v., in combination with HDM201.
Three to six patients will be treated until the determination of MTD(s)/RDE(s).
The starting dose for HDM201 is 60 mg.
Dose escalation and determination of the MTD/RDE for PDR001 with HDM201 will be guided by a BLRM with EWOC criteria. Dose escalation will be performed following the completion of two cycles of treatment. Safety assessments including adverse events (AEs) and laboratory values will be closely monitored for all enrolled patients in order to identify any DLTs. A single MTD/RDE will be defined; a disease-specific MTD/RDE will not be established.
Prior to the determination of the MTD/RDE a minimum of 12 patients must have been treated with the combinations of PDR001 and HDM201.
Paired tumor biopsies will be obtained from all patients. Analysis of these biopsy samples will contribute to a better understanding of the relationship between the dose and the pharmacodynamic activity of the combination.
Once the MTD/RDE has been declared for the combination therapy, the respective dose expansion part may begin. The main objective of the expansion part is to further assess the safety and tolerability of any study treatment at the MTD/RDE.
A key secondary objective is to assess changes in the immune infiltrate in tumor in response to treatment. This will be assessed in paired tumor biopsies collected from all patients, with a minimum of ten evaluable biopsy pairs (biopsy specimens must contain sufficient tumor for analysis), in patients treated at the MTD/RDE. If this is not feasible, collection of these biopsies may be stopped. A minimum of 20 patients are planned to be treated, however to account for failure of some biopsy specimens, approximately 30 patients are therefore estimated to be treated in each investigational arm. The secondary objectives include assessment of the preliminary anti-tumor activity.
In each treatment group a maximum of approximately six patients who have received and progressed on prior PD-1/PDL-1 inhibitor therapy may be enrolled. This number may be increased if a combination shows promise of overcoming resistance to prior treatment with single agent PD-1/PDL-1 inhibitors or if enrollment of patients naive to prior PD-1/PDL-1 inhibitor treatment is logistically unfeasible.
All patients enrolled in escalation part and expansion part may participate in the following study periods:
Prescreening period
Screening period
Treatment period 1
Treatment interruption period
Treatment period 2
Safety follow up period
Disease progression follow up
   Each study period is described below and shown in FIG. 31. All patients are considered “on-study” until they complete the safety follow up period, withdraw consent, are lost to follow up or death.
   The molecular pre-screening informed consent must be signed prior to any molecular pre-screening procedure (not applicable if TP53 status was already assessed outside of the study). Potential eligible patients must have documentation on their TP53 status through sequencing before the patient can be considered for full screening. A patient will be considered eligible for full screening if her/his tumor sample does not present mutation in exons 5, 6, 7 and 8 of TP53 gene, and if this TP53 status was obtained from a tumor sample collected no longer than 36 months before the first dose of study treatment (also applicable if TP53 wt status was obtained locally outside of the study). Exception: prior documentation (irrespective of date) of HDM2 amplification (defined as >4 copy number) does not require TP53 WT status confirmation.
   Screening tests should only begin after TP53 status is known.
   The screening period begins once the patient has signed the study informed consent. Patients will be evaluated to ensure that they meet all the inclusion and none of the exclusion criteria.
   Treatment period 1 will begin, following screening, on Cycle 1 Day 1. Patients will undergo clinical assessments at scheduled visits.
   Study treatment during treatment period 1 will be administered for six cycles of therapy unless the patient experiences unacceptable toxicity has clinical evidence of disease progression, and/or treatment is discontinued at the discretion of the investigator or the patient. Patients who have radiological evidence of disease progression but have evidence of clinical benefit may continue study treatment to complete six cycles following documented approval from Novartis.
   If a patient permanently discontinues study treatment during Treatment period 1 an End of Treatment visit must occur and appropriate follow-up assessments as defined below.
   Once a patient completes cycle 6 (treatment period 1), study treatment will be interrupted and the patient will enter the study treatment interruption period. Patients will continue study visits for safety assessments (monthly), tumor assessments (every 2 months), and collection of samples for PDR001 PK (monthly) and RO assessment (monthly). Once a patient has clinical or radiological evidence of disease progression, they may resume treatment following a documented discussion with Novartis.
   If a patient permanently discontinues study treatment rather than entering treatment period 2, an End of Treatment visit must occur and appropriate follow-up assessments must be performed as defined below.
   Patients should resume study treatment at the same dose and schedule they were receiving at the time of their treatment interruption (FIG. 27). Patients will initiate therapy in treatment period 2 only after documented agreement between the investigator and Novartis medical monitor that the patient is appropriate for treatment with regards to emergent toxicities and progression-related decline in clinical status. All patients must have a tumor assessment prior to resuming study treatment; this tumor assessment will be used as treatment period 2 baseline (FIG. 27). Following the completion of two cycles of study treatment, if a patient has not experienced any >grade 2 study treatment-related toxicities, he/she may continue on study under a reduced schedule of assessments per the institutions standard of care or every three months, whichever is more frequent. Patients who have radiological evidence of disease progression during treatment period 2 and have evidence of clinical benefit may continue study treatment following a documented discussion with Novartis.
   Following permanent discontinuation of study treatment in Treatment period 2, the End of Treatment visit and the safety follow-up assessments must occur as defined below.
   An EOT visit will occur within 14 days of the decision to permanently discontinue study treatment. All participating patients must complete the EOT visit.
   All patients will be followed for safety evaluations for 150 days following permanent discontinuation of PDR001.

Patient Population

The study will be conducted in adult patients with advanced/metastatic CRC or RCC.

### Inclusion Criteria:

Patients eligible for inclusion in this study have to meet all of the following criteria:
1. Written informed consent must be obtained prior to any procedures
2. Age ≥18 years.
3. Patients with advanced/metastatic cancer, with measurable disease as determined by RECIST version 1.1, who have progressed despite standard therapy or are intolerant to standard therapy, or for whom no standard therapy exists.
Patients must fit into one of the following groups for PDR001 in combination with HDM201
TP53 wild type CRC (not mismatch repair deficient by local assay including PCR and/or IHC) or TP53 wild type RCC
   To be considered TP53 wild-type a tumor must at a minimum have no mutations detected in exons 5, 6, 7 and 8 in a tumor sample collected no longer than 36 months before the first dose of study drug. Tumors previously documented as having genomic amplification of HDM2 (defined as >4 copy number, irrespective of the date) do not require TP53 WT status confirmation.

### 4. ECOG Performance Status ≤1

Patient must have a site of disease amenable to biopsy, and be a candidate for tumor biopsy according to the treating institution's guidelines. Patient must be willing to undergo a new tumor biopsy at screening, and again during therapy on this study.
5. Prior therapy with PD-1/PDL-1 inhibitors is allowed provided any toxicity attributed to prior PD-1- or PD-L1-directed therapy did not lead to discontinuation of therapy.

### Exclusion Criteria:

Patients eligible for this study must not meet any of the following criteria (inter alia):
Patient having out of range laboratory values defined as:
Creatinine clearance (calculated using Cockcroft-Gault formula, or measured)<40 mL/min
Total bilirubin >1.5×ULN, except for patients with Gilbert's syndrome who are excluded if total bilirubin >3.0×ULN or direct bilirubin >1.5×ULN
Alanine aminotransferase (ALT) >3×ULN, except for patients that have tumor involvement of the liver, who are excluded if ALT >5×ULN
Aspartate aminotransferase (AST) >3×ULN, except for patients that have tumor involvement of the liver, who are excluded if AST >5×ULN
Absolute neutrophil count <1.0×109/L without growth factor or transfusion support
Platelet count <75×109/L without growth factor or transfusion support
Hemoglobin (Hgb)<9 g/dL
Potassium, magnesium, calcium or phosphate abnormality >CTCAE grade 1 despite appropriate replacement therapy
   Patients who require the following treatments:
moderate to strong CYP3A4 inhibitors
any substrates of CYP3A4/5 with a narrow therapeutic index
   Moderate to strong CYP3A4 inducers
   Patients having out of range values for:
Absolute neutrophil count (ANC)<1500/μL
Platelets <100 000/μL

Treatment

The RP2D for PDR001 was established in the CPDR001X2101 phase I/II clinical study as 400 mg administered every four weeks, and will be used for all patients in this combination study
Therefore, patients will be treated with PDR001 at the RP2D of 400 mg Q4W. PDR001 (supplied as 100 mg powder for solution for infusion) will be administered by i.v. as a 30 minute infusion, or up to two hours if clinically indicated.
HDM201 will be given on day 1 (dl) and day 8 (d8) of a 4 week treatment cycle (q4w), i.e. regimen 1B. HDM201 will be supplied as hard gelatin capsules for oral administration in dosage strengths of 10 mg and 100 mg (expressed in mg of HDM201 free base). The capsules are differentiated by different size and/or color, and will be supplied in open-label, child-resistant, sealed bottles. Start dose will be 60 mg. The dose may be escalated in dose increments of 20 mg, e.g. 80 mg, 100 mg, 120 mg. HDM201 can be de-escalated below the proposed starting dose, e.g. 40 mg.
The C HDM201X2101 clinical study established the RDE for patients with solid tumors of 120 mg given on D1 and D8 of each 28 day cycle.
For this combination study, the starting dose will be 60 mg on D1 and D8 of each 28 day cycle. This dose is half of the RDE for patients with solid tumors, and although it has not been tested in patients, this dose and schedule it is expected to be active, as assessed by the induction of thrombocytopenia in patients with solid tumors treated with HDM201 at 15 mg-25 mg QD, 1 week on/3 weeks off.
PDR001 will be administered in combination with HDM201. Patients will be dosed on a flat scale and not by body weight or body surface area. Dosing of combination drug will occur immediately after completion of the PDR001 infusion during clinic visits.
On the days of pharmacokinetic sampling, the patients should take their morning doses at the clinic after pre-dose blood draws and PDR001 administration.
HDM201 should be administered orally on an empty stomach at least 1 hour before or 2 hours after a meal. The patient should take the capsules in the morning, at approximately the same time each day of dosing, with a glass of water and without chewing the capsules. If the patient is assigned to a dose level where multiple capsules are to be taken, the capsules should be taken consecutively, within as short an interval as possible. On the visit days, the patient will take HDM201 at the clinic under the supervision of the investigator or designee. If a patient forgets to take the dose as planned at day 8, he/she should take the dose as soon as possible. However, if more than 6 days have passed from the planned dose, then this dose should be skipped.
For HDM201, use of anti-coagulant therapy and anti-platelet agents should be carefully considered for patients with thrombocytopenia.

Study Drugs

### PDR001:

Pharmaceutical form: powder for solution for infusion.
For intravenous (IV) use. The antibody will be administered at a flat dose of 400 mg Q4W i.v. (intravenously) which is the single agent RDE (Recommended dose for expansion). The antibody may also be administered 300 mg i.v. Q3W for combination treatment regimens for which this may be more convenient.

### HDM201:

The drug product consists of HDM201 succinic acid drug substance filled directly into hard gelatin capsules (HGC), and does not contain any other excipients. The drug product is provided in four dosage strengths: 1 mg, 2.5 mg, 10 mg and 100 mg (based on the weight of the free form), intended for oral use. The 1 mg strength capsule is a “Size 3” yellow HGC, the 2.5 mg strength capsule is a “Size 3” Swedish Orange HGC, the 10 mg strength capsule is a “Size 1” Grey HGC, and the 100 mg is a “Size 0” Swedish Orange HGC. The drug product is packaged in child resistant, induction sealed High Density Polyethylene (HDPE) bottles.
For oral use.

### INCORPORATION BY REFERENCE

Other embodiments and examples including figures and tables are disclosed in International Patent Application Publication No. WO 2015/112900 and U.S. Patent Application Publication No. US 2015/0210769, entitled “Antibody Molecules to PD-1 and Uses Thereof,” which are incorporated by reference in its entirety.

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below.

The full scope of the invention should be determined by reference to the claims, along with their full scope of equivalents, and the specification, along with such variations.

1. A pharmaceutical combination comprising
(A) a HDM2 inhibitor which is (6S)-5-(5-Chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one (COMPOUND A) or pharmaceutically acceptable salt, solvate, complex or co-crystal thereof, 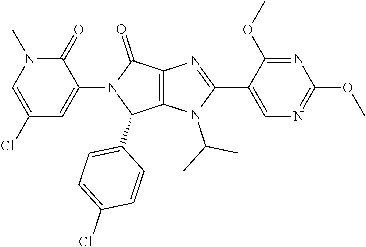 [SMILES: COc1ncc(-c2nc3c(n2C(C)C)[C@H](c2ccc(Cl)cc2)N(c2cc(Cl)cn(C)c2=O)C3=O)c(OC)n1]
and
(B) an anti-PD-1 antibody molecule which is an isolated antibody molecule capable of binding to a human Programmed Death-1 (PD-1) comprising a heavy chain variable region (VH) comprising a HCDR1, a HCDR2 and a HCDR3 amino acid sequence of BAP049-Clone-B or BAP049-Clone-E as described in Table 1 and a light chain variable region (VL) comprising a LCDR1, a LCDR2 and a LCDR3 amino acid sequence of BAP049-Clone-B or BAP049-Clone-E as described in Table 1.

2. The pharmaceutical combination of claim 1, wherein the anti-PD-1 antibody molecule comprises:
(a) a heavy chain variable region (VH) comprising a HCDR1 amino acid sequence of SEQ ID NO: 4, a HCDR2 amino acid sequence of SEQ ID NO: 5, and a HCDR3 amino acid sequence of SEQ ID NO: 3; and a light chain variable region (VL) comprising a LCDR1 amino acid sequence of SEQ ID NO: 13, a LCDR2 amino acid sequence of SEQ ID NO: 14, and a LCDR3 amino acid sequence of SEQ ID NO: 33;
(b) a VH comprising a HCDR1 amino acid sequence of SEQ ID NO: 1; a HCDR2 amino acid sequence of SEQ ID NO: 2; and a HCDR3 amino acid sequence of SEQ ID NO: 3; and a VL comprising a LCDR1 amino acid sequence of SEQ ID NO: 10, a LCDR2 amino acid sequence of SEQ ID NO: 11, and a LCDR3 amino acid sequence of SEQ ID NO: 32;
(c) a VH comprising a HCDR1 amino acid sequence of SEQ ID NO: 4, a HCDR2 amino acid sequence of SEQ ID NO: 5, and a HCDR3 amino acid sequence of SEQ ID NO: 3; and a VL comprising a LCDR1 amino acid sequence of SEQ ID NO: 13, a LCDR2 amino acid sequence of SEQ ID NO: 14, and a LCDR3 amino acid sequence of SEQ ID NO: 33; or
(d) a VH comprising a HCDR1 amino acid sequence of SEQ ID NO: 1; a HCDR2 amino acid sequence of SEQ ID NO: 2; and a HCDR3 amino acid sequence of SEQ ID NO: 3; and a VL comprising a LCDR1 amino acid sequence of SEQ ID NO: 10, a LCDR2 amino acid sequence of SEQ ID NO: 11, and a LCDR3 amino acid sequence of SEQ ID NO: 32.

3. The pharmaceutical combination according to claim 1 or 2, wherein the HDM2 inhibitor, or a pharmaceutically acceptable salt, solvate, complex or co-crystal thereof, and the anti-PD-1 antibody molecule are administered separately, simultaneously or sequentially.

4. The pharmaceutical combination of claim 1 or 2 wherein the HDM2 inhibitor is in oral dosage form.

5. The pharmaceutical combination of claim 1 or 2 wherein the anti-PD-1 antibody molecule is in injectable dosage form.

6. A pharmaceutical composition comprising the pharmaceutical combination according to any one of the preceding claims and at least one pharmaceutically acceptable carrier.

7. The pharmaceutical combination according to any one of claims 1 to 5 or the pharmaceutical composition according to claim 6 for use in the treatment of a proliferative disease.

8. Use of a pharmaceutical combination according to any one of claims 1 to 5 for the preparation of a medicament for the treatment of a proliferative disease.

9. A method for treating a proliferative disease in a subject in need thereof comprising administering to the subject the pharmaceutical combination according to any one of claims 1 to 5 or the pharmaceutical composition according to claim 6.

10. The pharmaceutical combination for use according to claim 7 or the use of a pharmaceutical combination according to claim 8 or the method according to claim 9, wherein the proliferative disease is a TP53 wildtype solid tumor.

11. The pharmaceutical combination for use according to claim 10, or the use of a pharmaceutical combination according to claim 10, or the method according to claim 10, wherein the proliferative disease is a renal cell carcinoma (RCC).

12. The pharmaceutical combination for use according to claim 10, or the use of a pharmaceutical combination according to claim 10, or the method according to claim 10, wherein the proliferative disease is a colorectal cancer (CRC).

13. The pharmaceutical combination for use according to claim 10, or the use of a pharmaceutical combination according to claim 10, or the method according to claim 10, wherein the proliferative disease is microsatellite stable colorectal cancer (MSS-CRC).

14. The pharmaceutical combination for use according to any one of claims 10 to 13, or the use of a pharmaceutical combination according to any one of claims 10 to 13, or the method according to any one of claims 10 to 13, wherein the HDM2 inhibitor is administered on day 1, and on either one of days 6 to 14 of a 4 week treatment cycle, preferably on day 1 and on either one of days 6 to 10 of a 4 week treatment cycle, more preferably on day 1 and day 8, of a 4 week treatment cycle (d1d8q4w).

15. The pharmaceutical combination for use according to any one of claims 10 to 14, or the use of a pharmaceutical combination according to any one of claims 10 to 14, or the method according to any one of claims 10 to 14, wherein the daily dose of the HDM2 inhibitor is selected from about 30, 40, 50, 60, 70, 80, 90, 100, 110, 120 mg, preferably the daily dose of the HDM201 inhibitor is from about 30 to about 120 mg, preferably the daily dose is from about 40 to about 120 mg, more preferably the daily dose is from about 60 to about 120 mg, wherein the daily dose amounts in mg refer to the HDM2 inhibitor as free form.

16. The pharmaceutical combination for use according to any one of claims 10 to 14, or the use of a pharmaceutical combination according to any one of claims 10 to 14, or the method according to any one of claims 10 to 14, wherein the daily dose of the HDM2 inhibitor is from about 60 to about 90 mg, even more preferably the daily dose is from about 60 to about 80 mg, wherein the daily dose amounts in mg refer to the HDM2 inhibitor as free form.

17. The pharmaceutical combination for use according to any one of claims 10 to 16, or the use of a pharmaceutical combination according to any one of claims 10 to 16, or the method according to any one of claims 10 to 16, wherein the anti-PD-1 antibody molecule is administered in a dose of about 300 mg to about 400 mg once every three weeks or once every four weeks.

18. The pharmaceutical combination for use according to any one of claims 10 to 17, or the use of a pharmaceutical combination according to any one of claims 10 to 17, or the method according to any one of claims 10 to 17, wherein the anti-PD-1 antibody molecule is administered at a dose of about 300 mg once every three weeks.

19. The pharmaceutical combination for use according to any one of claims 10 to 17, or the use of a pharmaceutical combination according to any one of claims 10 to 17, or the method according to any one of claims 10 to 17, wherein the anti-PD-1 antibody molecule is administered at a dose of about 400 mg once every four weeks.

20. The pharmaceutical combination for use according to any one of claims 1 to 5, or the pharmaceutical composition according to claim 6, or the use of a pharmaceutical combination according to claim 8 or the method according to claim 9, wherein the anti-PD-1 antibody molecule comprises:
(a) a heavy chain variable domain comprising the amino acid sequence of SEQ ID NO: 38 and a light chain variable domain comprising the amino acid sequence of SEQ ID NO: 42;
(b) a heavy chain variable domain comprising the amino acid sequence of SEQ ID NO: 38 and a light chain variable domain comprising the amino acid sequence of SEQ ID NO: 66;
(c) a heavy chain variable domain comprising the amino acid sequence of SEQ ID NO: 38 and a light chain variable domain comprising the amino acid sequence of SEQ ID NO: 70;
(d) a heavy chain variable domain comprising the amino acid sequence of SEQ ID NO: 50 and a light chain variable domain comprising the amino acid sequence of SEQ ID NO: 70;
(e) a heavy chain variable domain comprising the amino acid sequence of SEQ ID NO: 38 and a light chain variable domain comprising the amino acid sequence of SEQ ID NO: 46;
(f) a heavy chain variable domain comprising the amino acid sequence of SEQ ID NO: 50 and a light chain variable domain comprising the amino acid sequence of SEQ ID NO: 46;
(g) a heavy chain variable domain comprising the amino acid sequence of SEQ ID NO: 50 and a light chain variable domain comprising the amino acid sequence of SEQ ID NO: 54;
(h) a heavy chain variable domain comprising the amino acid sequence of SEQ ID NO: 38 and a light chain variable domain comprising the amino acid sequence of SEQ ID NO: 54;
(i) a heavy chain variable domain comprising the amino acid sequence of SEQ ID NO: 38 and a light chain variable domain comprising the amino acid sequence of SEQ ID NO: 58;
(j) a heavy chain variable domain comprising the amino acid sequence of SEQ ID NO: 38 and a light chain variable domain comprising the amino acid sequence of SEQ ID NO: 62;
(k) a heavy chain variable domain comprising the amino acid sequence of SEQ ID NO: 50 and a light chain variable domain comprising the amino acid sequence of SEQ ID NO: 66;
(l) a heavy chain variable domain comprising the amino acid sequence of SEQ ID NO: 38 and a light chain variable domain comprising the amino acid sequence of SEQ ID NO: 74;
(m) a heavy chain variable domain comprising the amino acid sequence of SEQ ID NO: 38 and a light chain variable domain comprising the amino acid sequence of SEQ ID NO: 78;
(n) a heavy chain variable domain comprising the amino acid sequence of SEQ ID NO: 82 and a light chain variable domain comprising the amino acid sequence of SEQ ID NO: 70;
(o) a heavy chain variable domain comprising the amino acid sequence of SEQ ID NO: 82 and a light chain variable domain comprising the amino acid sequence of SEQ ID NO: 66; or
(p) a heavy chain variable domain comprising the amino acid sequence of SEQ ID NO: 86 and a light chain variable domain comprising the amino acid sequence of SEQ ID NO: 66.

21. An anti-PD-1 antibody for use in treating a TP53 wildtype solid tumor, wherein the anti-PD-1 antibody is prepared for administration separately, simultaneously, or sequentially with a HDM2 inhibitor.

22. An anti-PD-1 antibody for use in treating TP53 wildtype RCC, wherein the anti-PD-1 antibody is prepared for administration separately, simultaneously, or sequentially with a HDM2 inhibitor.

23. An anti-PD-1 antibody for use in treating TP53 wildtype CRC, wherein the anti-PD-1 antibody is prepared for administration separately, simultaneously, or sequentially with a HDM2 inhibitor.

24. An anti-PD-1 antibody for use in treating TP53 wildtype MSS CRC, wherein the anti-PD-1 antibody is prepared for administration separately, simultaneously, or sequentially with a HDM2 inhibitor.

25. A HDM2 inhibitor for use in treating a TP53 wildtype solid tumor, wherein the HDM2 inhibitor is prepared for administration separately, simultaneously, or sequentially with an anti-PD-1 antibody.

26. A HDM2 inhibitor for use in treating TP53 wildtype solid tumor in a patient, wherein the HDM2 inhibitor is prepared for administration separately, simultaneously, or sequentially with an anti-PD-1 antibody and wherein the patient has received previous immuno-therapy.

27. A combined preparation comprising (a) one or more dosage units of a HDM2 inhibitor according to claim 1, or a pharmaceutically acceptable salt, solvate, complex or co-crystal thereof, and (b) one or more dosage units of an anti-PD-1 antibody according to claim 2, and at least one pharmaceutically acceptable carrier.

28. A commercial package kit comprising as active ingredients the pharmaceutical combination according to any one of claims 1 to 5 together with instructions for simultaneous, separate or sequential administration of said pharmaceutical combination to a patient in need thereof for use in the treatment of a proliferative disease.
